(19) United States
(12) Patent Application Publication
(10) Pub. No.: US 2004/0009604 A1
(43) Pub. Date: Jan. 15, 2004

(54) Potent oncolytic herpes simplex virus for cancer therapy

(76) Inventors: Xiaoliu Zhang, Houston, TX (US); Xinping Fu, Zhuhai (CN)

Correspondence Address: FULBRIGHT & JAWORSKI, LLP, 1301 MCKINNEY, SUITE 5100, HOUSTON, TX, 77010-3095, US

(21) Appl. No.: 10/397,635

(22) Filed: Mar. 26, 2003

Related U.S. Application Data
(60) Provisional application No. 60/367,788, filed on Mar. 27, 2002.
(60) Provisional application No. 60/410,024, filed on Sep. 11, 2002.

(51) Int. Cl. C12N 15/86; C12N 7/00

(52) U.S. Cl. 435/456; 435/235.1; 435/320.1; 530/395

(57) ABSTRACT

The present invention is directed to an oncolytic Herpes Simplex Virus having multiple cell membrane fusion mechanisms and preferably comprising a strict late viral promoter for effective conditional replication, such as in a malignant cell. In specific embodiments, the cell membrane fusion mechanisms are either from a mutant virus generated through random mutagenesis or through insertion of a fusogenic membrane glycoprotein, and in further specific embodiments the strict late viral promoter UL38p regulates expression of the glycoprotein.

The present invention claims priority to U.S. Provisional Patent Application Serial No. 60/367,788, filed Mar. 27, 2002 and No. 60/410,024, filed Sep. 11, 2002, both of which are incorporated by reference herein in their entirety.

The present invention was developed at least in part using funds provided by the United States Government pursuant to NIH grant number CA 58204. The government may have certain rights in the invention.

### FIELD OF THE INVENTION

The present invention is directed to the fields of virology, cancer biology, and medicine. Specifically, the present invention regards compositions and methods directed to an oncolytic vector utilizing viral infection and cell membrane fusion mechanisms. More specifically, a Herpes Simplex Virus (HSV) vector comprising multiple fusogenic mechanisms is utilized, and, in some embodiments, further comprises a strict late viral promoter.

### BACKGROUND OF THE INVENTION

Replication selective oncolytic viruses have shown great promise as anti-tumor agents for solid tumors. The viruses have been constructed genetically so that they are able to preferentially replicate within tumor cells, while being restricted in their ability to replicate in normal cells. The principle anti-tumor mechanism of oncolytic viruses is through a direct cytopathic effect as they propagate and spread from initially infected tumor cells to surrounding tumor cells, achieving a larger volume of distribution and anticancer effects. Oncolytic herpes simplex virus (HSV) were initially designed and constructed for the treatment of brain tumors (Andreansky et al., 1996). Subsequently, they have been found to be effective in a variety of other human solid tumors, including breast (Toda et al., 1998), prostate (Walker et al., 1999), lung (Toyoizumi et al., 1999), ovarian (Coukos et al., 1999), colon and liver cancers (Carroll et al., 1996; Pawlik et al., 2000). The safety of oncolytic HSVs has also been extensively tested in mice (Sundaresan et al., 2000) and primates (Aotus), which are extremely sensitive to HSV infection (Todo et al., 2000). The studies have confirmed that oncolytic HSVs are extremely safe for in vivo administration.

Despite these encouraging preclinical studies, results from early clinical trials have suggested that the current versions of oncolytic viruses, although safe, may only have limited anti-tumor activity on their own (Markert et al., 2000; Rampling et al., 2000; Nemunaitis et al., 2001). One of the main reasons for the sub-optimal oncolytic efficacy is probably because viral gene deletions that confer tumor selectivity also result in reduced potency of the virus in tumors. For example, the complete elimination of endogenous γ34.5 function from HSV, one of the common approaches for the construction of oncolytic HSV, significantly reduces viral replication potential and therefore may compromise the ability of the virus to spread within the targeted tumors (Kramm et al., 1997). Therefore, strategies designed to further enhance the potency of current oncolytic viruses will likely increase their chance of clinical success.

Recently it has been reported that the envelop fusogenic membrane glycoproteins (FMGs) from certain viruses are extremely potent at killing tumor cells (Bateman et al., 2000). An example of one such FMGs is a C-terminally truncated form of the gibbon ape leukemia virus envelope glycoprotein (GALV.fus) (Bateman et al., 2000; Fielding et al., 2000). This truncated form of FMG lacks the 16 amino acid R-peptide of the wild-type protein, which normally serves to restrict fusion of the envelope until it is cleaved during viral infection (Januszeski et al., 1997). This alteration in the FMG renders the protein constitutively highly fusogenic to human cells that express the Pit-1 receptor for GALV. Transduction of the GALV.fus into a range of human tumor cells efficiently kills the cells, through a process of syncytial formation (Higuchi et al., 2000). In addition, the bystander killing effect is at least one log higher using GALV.fus than the suicide genes HSVtk or cytosine deaminase (Diaz et al., 2000; Higuchi et al., 2000). However, it is expected that this gene has to be efficiently delivered into tumor cells in a controlled fashion so that its potential therapeutic benefit can be materialized.

Clinical application of therapeutic genes such as GALV.fus requires their tumor selection expression. One way to achieve this is to use tumor or tissue-specific transcriptional regulatory elements to control the expression of a therapeutic gene of interest. Although several tissue-specific promoters have been shown to direct gene expression selectively in the tumor cells of the same tissue origin (Huber et al., 1991; Vile and Hart, 1993; Latham et al., 2000; Tanaka et al., 2000), their activity is usually much weaker than the constitutive viral promoters such as the cytomegalovirus immediate early promoter (CMV-P) and the long terminal repeat (LTR) of retroviruses (Schuur et al., 19996; Koga et al., 2000; Latham et al., 2000), resulting in poor anti-tumor efficacy. Strategies such as adding CMV enhancer sequences to the upstream of tissue specific promoters can substantially increase the promoter activity (Latham et al., 2000). However, this action also causes the original promoters to lose their tissue-specificity. Another potential concern for these tissue-specific promoters is that they also intend to lose their tissue specificity once they are cloned into viral vectors (Babiss et al., 1987).

Like many DNA viruses, the transcriptional program of HSV-1 is a regulated cascade in which early and late phases of gene expression are separated by viral DNA synthesis (Wagner et al., 1995). The early genes are transcribed prior to viral DNA replication, while late genes are expressed at high levels only after viral DNA replication has taken place. Late transcripts can be further categorized as either leaky-late, which are readily detectable prior to the onset of viral genome replication, or strict late that are only reliably detectable after the onset of viral DNA replication (Holland et al., 1980; Johnson and Everett, 1986; Flanagan et al., 1991).

The present invention in some embodiments uses a strict late viral promoter (e.g., the promoter of the UL38 gene of HSV) to selectively express a therapeutic nucleic acid sequence (e.g., GALV.fus) in tumor tissues. This is because in the context of an oncolytic HSV, such a strict late viral promoter is extremely active in the tumor tissue where the oncolytic virus can fully replicate, but silent in the normal cells, if these are non-dividing or post-mitotic, since viral replication would be limited.

WO 01/45737 is directed to a mutant human herpes simplex virus lacking a functionally active wild-type glycoprotein C polypeptide encoding UL44 and preferably oncolytic to a neoplasm. In preferred embodiments, the virus is deficient in the viral particle attachment to a cell susceptible to its effects or in attachment to the cell surface through a receptor.

WO 98/40492 addresses a nucleic acid vector for therapy of a malignant disease wherein the vector directs expression of a syncytium-inducing polypeptide on a eukaryotic cell surface. In specific embodiments the syncytium-inducing polypeptide is a viral membrane glycoprotein, and in other specific embodiments the nucleic acid vector is a defective HSV.

The present invention addresses a deficiency in the art by providing a conditionally replicating (oncolytic) HSV containing different membrane fusion mechanisms. These include selection of fusogenic oncolytic HSV through random mutagenesis of the virus and insertion of fusogenic glycoproteins into the oncolytic virus. Unlike the technology described in WO 98/40492 where the vector is defective, the antitumor activity from the present invention comes from two completely different but complementary mechanisms (direct viral oncolysis from a conditionally replicating viral vector and cell membrane fusion), wherein, preferably, the vector is non-defective. Such a combined approach can provide many advantages over the embodiments in WO 98/40492 at treating malignant diseases. For example, the combination of membrane fusion activity with a conditionally replicating viral vector generates a syngeneic antitumor effect, as the syncytia formation from membrane fusion can facilitate the spread of oncolytic virus in the tumor tissues. This should also reduce the occurrence of virus-resistant tumor cells, because those cells that become resistant to one mechanism (e.g. oncolytic virus infection/replication) may be indirectly destroyed by the other tumor-destroying mechanism (e.g. syncytial formation). More importantly, having two or more mechanisms to provide cell membrane fusion capacity will render the composition effective for different populations of cells, such as, for example, those having different types of viral attachment receptors. Finally, the present invention also provides a way for selective expression of the fusion protein in tumor cells, therefore directly increasing the clinical safety of the therapeutic approach. Uncontrolled expression of the fusion peptide, as embodied in WO 98/40492, would potentially cause widespread damage to normal tissues in patients.

### SUMMARY OF THE INVENTION

The present invention addresses a long-felt need in the art by providing a potent oncolytic HSV for therapy of undesirable cells, such as malignant cells. In preferred embodiments, the conditionally replicating HSV comprises at least two mechanisms for generating cell membrane fusion to rid a culture, tissue or organism of at least some undesirable cells, to inhibit proliferation of at least some undesirable cells, to prevent proliferation of at least some desirable cells, or a combination thereof.

Incorporation of cell membrane fusion capability into an oncolytic HSV can significantly increase the antitumor potency of the virus (Fu and Zbang, 2002; Fu et al., 2002). Fusogenic oncolytic HSVs utilized in the present invention may be generated by any means so long as they comprise cell fusogenic activity. In specific embodiments, the vector is generated by one of the following procedures: 1) screening for syncytial phenotype from any vector, such as a well-established oncolytic HSV, following random mutagenesis (as described, for example, for the creation of Fu-10 (Fu and Zhang, 2002)); 2) insertion of a nucleic acid sequence encoding a gene product comprising fusogenic properties into the vector, such as the nucleic acid sequence encoding the hyperfusogenic membrane glycoprotein of gibbon ape leukemia virus (GALV.fus) into the genome of an oncolytic HSV (such as, for the creation of Synco-2 (Fu et al., 2002)); and 3) incorporation of both of these two membrane fusion mechanisms into a single oncolytic HSV (such as, for example, for the generation of Synco-2D). In any case, the fusogenic oncolytic HSVs showed a dramatically enhanced antitumor activity when compared with the non-fusogenic virus.

In one aspect, an HSV is rendered fusogenic by generating mutations or other manipulations of the virus to attain that function. To further enhance the anti-tumor potency of the virus, it is engineered to comprise a nucleic acid sequence encoding a fusogenic polypeptide, such as GALV.fus, and/or to engineer a characteristic, such as a mutation that confers fusogenic properties. Moreover, in specific embodiments, expression of a nucleic acid sequence, such as a GALV.fus, in a tumor-specific manner is useful. In one embodiment, the tumor-specific expression is through a strictly late viral promoter whose activity is dependent on the ability of the oncolytic virus to replicate in tumor cells. It restricts the GALV.fus expression to tumor tissues only, and any nucleic acid sequence of the present invention may be regulated by a strictly late viral promoter. In one embodiment, the construction of such oncolytic HSVs through an enforced ligation strategy is obtained, and in vitro characterization and in vivo evaluation of these viruses in xenografted human tumor is described.

In certain embodiments of the present invention, the transcriptional regulatory elements of strict late genes is useful as strong and tumor-specific promoters when utilized with an oncolytic HSV. Such a strict late viral promoter is extremely active in the tumor tissue where the oncolytic virus can fully replicate but is silent in non-dividing or post-mitotic normal cells since viral replication would be limited.

There is an oncolytic HSV that contains the secreted form of alkaline phosphatase gene (SEAP) driven by the promoter of UL38, a well-characterized strict late gene of HSV (Goodart et al., 1992; Guzowski and Wagner, 1993; Guzowski et al., 1994). This promoter has very low activity in non-dividing cells. However, in cycling cells and in the presence of lytic HSV infection, its activity is dramatically increased to a level equivalent to that of the CMV-P. In vivo administration of an oncolytic HSV containing this promoter cassette also demonstrated strong tumor-selective expression property. Hence, a strict late viral promoter in an oncolytic HSV can function as a strong tumor-selective promoter.

In other embodiments of the present invention, a strict late viral promoter is used in the context of an oncolytic virus as a tumor-specific promoter. A skilled artisan recognizes the virus could be any kind of virus that can be developed for oncolysis, including retrovirus, adenovirus, or adeno-associated virus.

In an object of the present invention, there is a composition comprising a cell membrane fusion-generating Herpes Simplex Virus vector comprising at least one additional cell membrane fusion-generating mechanism. In a specific embodiment, the HSV vector is conditionally replicating. In a further specific embodiment, the conditionally replicating is defined as the vector comprising a strict late viral promoter. In a specific embodiment, the cell membrane fusion-generating vector is generated by mutagenesis of a non-cell membrane fusion-generating vector. In a specific embodiment, the additional cell membrane fusion mechanism comprises a nucleic acid sequence in the HSV vector that encodes a fusogenic polypeptide. In a specific embodiment, the fusogenic polypeptide is further defined as a membrane glycoprotein. In a specific embodiment, the membrane glycoprotein is paramyxovirus F protein, HIV gp160 protein, SIV gp160 protein, retroviral Env protein, Ebola virus Gp, or the influenza virus haemagglutinin. In a specific embodiment, the glycoprotein is a membrane glycoprotein from gibbon ape leukemia virus (GALV).

In a specific embodiment, the glycoprotein is a C-terminally truncated form of the gibbon ape leukemia virus envelope glycoprotein (GALV.fus). In a specific embodiment, the expression of the nucleic acid sequence is controlled by a strict late viral promoter. In a specific embodiment, the strict late viral promoter is the promoter of UL38 or Us11 of HSV. In a specific embodiment, further comprises a pharmaceutically acceptable excipient.

In another object of the present invention, there is a method of generating fusion between a first cell and a second cell, comprising the step of introducing to the first cell a cell membrane fusion-generating Herpes Simplex Virus vector comprising at least one additional cell membrane fusion-generating mechanism, wherein following the introduction step the cell membrane of the first cell fuses with the cell membrane of the second cell. In a specific embodiment, the first cell, second cell, or both first and second cells are malignant cells. In a specific embodiment, the step is repeated with a plurality of cells. In a specific embodiment, the HSV vector is conditionally replicating. In a specific embodiment, the additional cell membrane fusion mechanism comprises a nucleic acid sequence encoding a fusogenic polypeptide. In a specific embodiment, the expression of the nucleic acid sequence is regulated by a strict late viral promoter. In a specific embodiment, the strict late viral promoter is the promoter of UL38 or Us11 of HSV.

In an additional object of the present invention, there is a method of destroying a malignant cell, comprising the step of introducing to the cell a cell membrane fusion-generating Herpes Simplex Virus vector comprising at least one additional cell membrane fusion-generating mechanism, wherein following said introduction the membrane of the malignant cell fuses with another cell membrane. In a specific embodiment, the malignant cell is of human origin. In a specific embodiment, the introduction step is further defined as administering the vector to a human at about 1×10⁹ plaque forming units (pfu). In a specific embodiment, the method further comprises administrating an additional cancer therapy to the human. In a specific embodiment, the additional cancer therapy is chemotherapy, radiation, surgery, immunotherapy, gene therapy, or a combination thereof.

In an additional object of the present invention, there is a composition comprising an oncolytic virus, wherein the virus comprises a strict late viral promoter. In a specific embodiment, virus is further defined as being tumor-specific.

In an embodiment of the present invention, there is a composition, comprising a cell membrane fusion-generating Herpes Simplex Virus vector, said vector including at least one additional cell membrane fusion-generating component. In one aspect of the present invention, the HSV vector is conditionally replicating, such as when the vector comprises a strict late viral promoter. The cell membrane fusion-generating vector may be a non-cell membrane fusion-generating vector that further comprises a mutation. In an embodiment of the present invention, the additional cell membrane fusion component comprises a nucleic acid sequence in the HSV vector that encodes a fusogenic polypeptide, which, in some embodiments, is further defined as a membrane glycoprotein. The membrane glycoprotein may be paramyxovirus F protein, HIV gp160 protein, SIV gp160 protein, retroviral Env protein, Ebola virus Gp, or the influenza virus haemagglutinin. In a specific embodiment, the glycoprotein is a membrane glycoprotein from gibbon ape leukemia virus (GALV) or is a C-terminally truncated form of the gibbon ape leukemia virus envelope glycoprotein (GALV.fus).

The expression of the nucleic acid sequence may be controlled by a strict late viral promoter, such as the promoter of UL38 or Us11 of HSV.

In some embodiments of the present invention, the compositions described herein further comprise a pharmaceutically acceptable excipient.

In an additional embodiment of the present invention, there is a method of generating fusion between a first cell and a second cell, comprising the step of fusing the second cell membrane with the first cell membrane by introducing to the first cell a cell membrane fusion-generating Herpes Simplex Virus vector comprising at least one additional cell membrane fusion-generating component. In specific embodiments, the first cell, second cell, or both first and second cells are malignant cells, such as liver cancer malignant cells, breast cancer malignant cells, ovarian cancer malignant cell, prostate cancer malignant cells, and/or lung cancer malignant cells. In specific embodiments, a step of the method is repeated with a plurality of cells.

The HSV vector may be conditionally replicating. Furthermore, the additional cell membrane fusion mechanism may comprise a nucleic acid sequence encoding a fusogenic polypeptide. In a specific embodiment, the expression of the nucleic acid sequence is regulated by a strict late viral promoter, such as the promoter of UL38 or Us11 of HSV.

In an additional embodiment of the present invention, there is a method of destroying a malignant cell, comprising the step of introducing to the cell a cell membrane fusion-generating Herpes Simplex Virus vector comprising at least one additional cell membrane fusion-generating mechanism, wherein following said introduction the membrane of the malignant cell fuses with another cell membrane. In a specific embodiment, the malignant cell is of human origin. The introduction step may be further defined as administering the vector to a human at about 1×10⁹ plaque forming units (pfu), and in some embodiments the method further comprises administrating an additional cancer therapy to the human, wherein the additional cancer therapy is chemotherapy, radiation, surgery, immunotherapy, gene therapy, or a combination thereof.

In another embodiment of the present invention, there is a composition comprising an oncolytic virus, wherein the virus comprises a strict late viral promoter. The virus may be further defined as being tumor-specific.

In an additional embodiment of the present invention, there is a method of generating a cell membrane fusion-generating Herpes Simplex Virus vector comprising the steps of introducing a mutation to a non-cell membrane fusion-generating Herpes Simplex Virus vector; and incorporating into the vector a nucleic acid sequence encoding a cell membrane fusion-generating polypeptide.

In another embodiment of the present invention, there is a composition comprising a Herpes Simplex Virus vector comprising a mutation that renders the vector a cell membrane fusion-generating vector; and a nucleic acid sequence encoding GALV.fus.

In an additional embodiment of the present invention, there is a method of destroying a malignant cell comprising introducing to said cell a composition comprising an oncolytic virus, wherein the virus comprises a strict late viral promoter.

In one embodiment of the present invention, there is a composition, comprising a vector comprising a first cell membrane fusion-generating activity; and a second cell membrane fusion-generating activity. The vector may be a Herpes Simplex Virus vector, and it may be conditionally replicating, such as being further defined as the vector comprising a strict late viral promoter. In a specific embodiment, the first cell membrane fusion-generating activity, the second cell membrane fusion-generating activity, or both comprise a mutation, said mutation conferring said cell membrane fusion-generating activity to the vector or a gene product encoded thereby. In another specific embodiment, the first cell membrane fusion-generating activity, the second cell membrane fusion-generating activity, or both comprise a nucleic acid sequence that encodes a fusogenic polypeptide, such as one that is further defined as a membrane glycoprotein, such as paramyxovirus F protein, HIV gp160 protein, SIV gp160 protein, retroviral Env protein, Ebola virus Gp, or the influenza virus haemagglutinin. The glycoprotein may be a membrane glycoprotein from gibbon ape leukemia virus (GALV) or a C-terminally truncated form of the gibbon ape leukemia virus envelope glycoprotein (GALV.fus). The expression of the nucleic acid sequence is controlled by a strict late viral promoter, in some embodiments, such as the strict late viral promoter is the promoter of UL38 or Us11 of HSV. In a specific embodiment, the composition further comprises a pharmaceutically acceptable excipient.

In another embodiment of the present invention, there is a method of generating fusion between a first cell and a second cell, comprising the step of fusing the second cell membrane with the first cell membrane by introducing to the first cell a vector comprising a first cell membrane fusion-generating activity and a second cell membrane fusion-generating activity. The first cell, second cell, or both first and second cells may be malignant cells, such as in a solid tumor and/or such as in a human. The introducing step may be further defined as delivering the vector to the human, such as systemically delivering the vector to the human, such as intravenously delivering the vector to the human. The step may be repeated with a plurality of cells. The vector may be a conditionally replicating Herpes Simplex Virus vector.

In a specific embodiment, the first cell membrane fusion-generating activity, the second cell membrane fusion-generating activity, or both comprise a mutation, said mutation conferring said cell membrane fusion-generating activity to the vector or a gene product encoded thereby. In another embodiment, the first cell membrane fusion-generating activity, the second cell membrane fusion-generating activity, or both comprise a nucleic acid sequence that encodes a fusogenic polypeptide. The expression of the nucleic acid sequence may be regulated by a strict late viral promoter, such as the promoter of UL38 or Us11 of HSV.

In a specific embodiment, the method further comprises the step of providing enhanced tumor antigen presentation compared to in the absence of said vector, and the enhanced tumor antigen presentation provides an improved antitumor immunity compared to in the absence of said enhanced tumor antigen presentation.

In an additional embodiment of the present invention, there is a method of destroying a malignant cell, comprising the step of introducing to the cell a vector comprising a first cell membrane fusion-generating activity; and a second cell membrane fusion-generating activity, wherein following said introduction the membrane of the malignant cell fuses with another cell membrane. In a specific embodiment, the malignant cell is in a human and/or the introduction step is further defined as administering at least about 1×10⁹ plaque forming units (pfu) of the vector to the human. The method may further comprise administering an additional cancer therapy to the human, such as chemotherapy, radiation, surgery, immunotherapy, gene therapy, or a combination thereof. The method may further comprise the step of providing enhanced tumor antigen presentation compared to in the absence of said vector, such as wherein the enhanced tumor antigen presentation provides an improved antitumor immunity compared to in the absence of said enhanced tumor antigen presentation.

In an embodiment of the present invention, there is a composition comprising an oncolytic virus, wherein the virus comprises a strict late viral promoter, and the virus may be further defined as being tumor-specific.

In another embodiment of the present invention, there is a method of generating a cell membrane fusion-generating Herpes Simplex Virus vector comprising the steps of introducing a mutation to a non-cell membrane fusion-generating Herpes Simplex Virus vector, said mutation conferring cell-membrane fusion-generating activity to the vector or a gene product encoded thereby; and incorporating into said vector a nucleic acid sequence encoding a cell membrane fusion-generating polypeptide.

In an additional embodiment of the present invention, there is a composition, comprising a Herpes Simplex Virus vector comprising a mutation that confers to the vector or a gene product encoded thereby a cell membrane fusion-generating activity; and a nucleic acid sequence encoding GALV.fus.

In a further embodiment of the present invention, there is a method of destroying a malignant cell comprising introducing to said cell a composition comprising an oncolytic virus, wherein the virus comprises a strict late viral promoter.

In a further embodiment of the present invention, there is a mammalian cell comprising a composition described herein.

In an additional embodiment of the present invention, there is a vector, comprising a first cell membrane fusion-generating activity and a second cell membrane fusion-generating activity, wherein said vector is obtainable by a method comprising at least one of the following steps generating a mutation in a nucleic acid sequence of the vector, wherein the mutation confers to the vector or a gene product encoded thereby the cell membrane fusion-generating activity; incorporating into the vector a nucleic acid sequence encoding a gene product comprising cell membrane fusion-generating activity; or both.

The incorporating step may be further defined as providing a first polynucleotide comprising a Herpes Simplex Virus genome, said Herpes Simplex Virus being non-infectious; providing a second polynucleotide comprising the nucleic acid sequence encoding at least one gene product comprising cell membrane fusion-generating activity; and at least one nucleic acid sequence encoding a gene product comprising a functional packaging signal; and incorporating the nucleic acid sequence encoding a gene product comprising cell membrane fusion-generating activity and the nucleic acid sequence encoding a gene product comprising a functional packaging signal into the first polynucleotide, wherein said incorporating step generates an infectious Herpes Simplex Virus.

The incorporating the nucleic acid sequence encoding a gene product comprising cell membrane fusion-generating activity and the nucleic acid sequence encoding a gene product comprising a functional packaging signal into the first polynucleotide step may be further defined as mixing the first and second polynucleotides together to form a mixture; introducing the mixture to a cell; and assaying for lysis of said cell.

The first polynucleotide may be provided on a bacterial artificial chromosome. The Herpes Simplex Virus of the first polynucleotide may comprise a deletion of γ34.5; a deletion of one or more copies of pac; or a combination thereof. The infectious Herpes Simplex Virus may be replication selective. The second polynucleotide may be provided on a plasmid. The expression of the nucleic acid sequence encoding at least one gene product comprising cell membrane fusion-generating activity may be regulated by CMV immediate early promoter.

In an additional embodiment of the present invention, there is a method of generating a vector comprising a first cell membrane fusion-generating activity and a second cell membrane fusion-generating activity, comprising at least one of the following steps generating a mutation in a nucleic acid sequence of the vector, wherein the mutation confers to the vector or a gene product encoded thereby the cell membrane fusion-generating activity; incorporating into the vector a nucleic acid sequence encoding a gene product comprising cell membrane fusion-generating activity; or both. The incorporating step may further be defined as providing a first polynucleotide comprising a Herpes Simplex Virus genome, said Herpes Simplex Virus being non-infectious; providing a second polynucleotide comprising the nucleic acid sequence encoding at least one gene product comprising cell membrane fusion-generating activity; and at least one nucleic acid sequence encoding a gene product comprising a functional packaging signal; and incorporating the nucleic acid sequence encoding a gene product comprising cell membrane fusion-generating activity and the nucleic acid sequence encoding a gene product comprising a functional packaging signal into the first polynucleotide, wherein said incorporating step generates an infectious Herpes Simplex Virus. In another embodiment, there is the vector obtained by a method described herein.

In another embodiment of the present inveniton, there is a method of increasing tumor antigen presentation in an individual, said individual comprising a malignant cell, comprising the step of providing to the individual a vector comprising a first cell membrane fusion-generating activity and a second cell membrane fusion-generating activity. The increased tumor antigen presentation provides an improved antitumor immunity in the individual compared to in the absence of said increased tumor antigen presentation, in some embodiments.

In another object of the present invention, there is a method of destroying a malignant cell comprising introducing to said cell a composition comprising an oncolytic virus, wherein the virus comprises a strict late viral promoter.

### BRIEF DESCRIPTION OF THE DRAWINGS

For a more complete understanding of the present invention, reference is now made to the following descriptions taken in conjunction with the following accompanying drawings.

FIG. 1 provides a schematic illustration of the enforced strategy for the insertion of GALV.fus gene into oncolytic HSV. The mutated HSV DNA sequence is represented by dotted gray bars and the BAC sequence is represented by black bars in fHSV-delta-pac. The pac and the gene cassette from shuttle plasmids are labeled. The gene insertion is done in vitro before the ligation mixture was transfected into Vero cells for the generation of infectious viruses.

FIG. 2 shows phenotypic characterization of Synco-1 on tumor cells. The top panel shows the tumor cells infected with Baco-1. Each photo contains a single infection focus. The bottom panel shows the cells infected with Synco-1. Each photo shows a single syncytium (original magnification, ×200).

FIGS. 3A and 3B illustrate increased tumor cell killing ability from Synco-1 infection. The percentage of cell survival was calculated by dividing the live cells from the infected wells with the total number of cells in the well that was not infected. Statistical comparison was made between the cells infected with Baco-1 or Synco-1 at each of the virus doses and the time points of harvest. *P<0.001.

FIG. 4 demonstrates enhanced oncolytic potency of Synco-1 in nude mouse—human tumor xenografts. Treatment groups include Synco-1, Baco-1 or PBS. Tumor growth ration was determined by dividing the tumor volume measured on the indicated week with the tumor volume at the start of injection. The figures represent the mean value±standard deviation (n=10 per group). P<0.001 when comparing with PBS control.

FIGS. 5A-5L provide phenotypic characterization of Synco-2 and selective GALV.fus expression from the virus. FIGS. 5A-5C show different tumor cells (FIG. 5A, U87 MG; FIG. 5B, DU 145; and FIG. 5C, Hep 3B) infected with Synco-2. FIGS. 5D and 5E show Hep 3B cells infected with Synco-1 (FIG. 5D) and Synco-2 (FIG. 5E) in the presence of ACV in the medium. FIG. 5F shows uninfected human embryonic fibroblasts HF 333.We. FIGS. 5G-5I shows different status of HF333.We infected with Synco-1 (FIG. 5G, shows cell maintained in growth medium; FIG. 5H shows cells serum-starved for 24 h; FIG. 51 shows serum-starved cells for 24 h plus incubation with lovastatin). FIGS. 5J-5L show different status of HF 333.We (FIG. 5J shows cells maintained in growth medium; FIG. 5K shows cells serum-starved for 48 h; FIG. 5L shows cells serum-starved plus incubation with lovastatin) infected with Synco-2, (Original magnification, ×200).

FIGS. 6A-6B show a comparison of anti-tumor potency between Synco-1 and Synco-2 and demonstration of their in vivo syncytial formation. FIG. 6A is a comparison of oncolytic potency on xenografted human tumors. Treatment groups include Baco-1, Synco-1, Synco-2 and PBS. FIG. 6B shows microscopic examination of tumor tissues after viral or PBS injection. The syncytial formation in Synco-1 or Synco-2 infected tumor tissues are indicated by arrow heads.

FIG. 7 is a schematic exemplary illustration of an enforced ligation strategy for the construction of oncolytic HSV containing AP gene. The plasmid DNA sequence is represented by filled area, and the HSV DNA sequence in the fHSV-delta-pac is represented by the hatched area. The BAC sequence, the HSV packaging signal (pac), the two different promoter elements, and the AP gene are each individually labeled (not proportional to their actual sizes). The locations of the restriction enzyme PacI site on each construct are also indicated. The gene cassettes containing AP gene were cut out with PacI and ligated into fHSV-delta-pac that has also been linearized with PacI. The ligation mixture was directly transfected into Vero cells for the generation of infectious viruses.

FIG. 8 illustrates in vitro characterization of UL38p cloned in a plasmid. Both pLox-AP and pIMJ-pac-AP were transfected into Vero cells, which were then infected with 0.1 pfu/cell of an oncolytic HSV (Baco-1) or mock infected (with medium only). The medium was collected 24 h after viral infection (i.e., 48 h after DNA transfection) and quantified for the AP secretion. The results represent the average of three independent experiments.

FIG. 9 shows in vitro characterization of UL38p in the context of oncolytic HSV. Human embryonic fibroblasts (HF 333.We) were seeded duplicately into 12-well plates at 1×10⁵ cells/well. One set of cells was treated with 20 μM lovastatin in serum-free medium for 30 h. Both untreated (in complete medium) and the lovastatin-arrested cells were then infected with either Baco-AP1 or Baco-AP2 at 0.1 pfu/cell. The supernatants were collected 24 h after infection and the AP in the medium was quantified. The figures represent the average of two independent experiments.

FIG. 10 demonstrates in vivo characterization of UL38p in the context of oncolytic HSV. Mice with established liver tumor on the right-hand side flank were intratumorally injected (i.t.) with 5×10⁶ pfu of either Baco-AP-1 or Baco-AP2. Mice without tumor were injected with the same amount of virus either intramuscularly (i.m.) or intravenously (i.v.). Blood was collected at the indicated day after virus inoculation and the AP secreted into the blood was quantified. Data are expressed as the mean±SE (n=5).

FIG. 11 provides a summary of the strategy for fusogenic oncolytic HSV. The large circle area represents the HSV DNA sequence including the BAC sequence represented by black circle in the fHSV-delta-pac. The pac and the gene cassette from shuttle plasmids are labeled. The gene cassettes were ligated into the BAC-HSV construct in vitro before the ligation mixture was transfected into Vero cells for the generation of infectious viruses. After that, to generate the enhanced fusogenic potent HSVs, random mutagenesis was performed.

FIGS. 12A-12F show in vitro phenotypic characterization of Synco-2D in ovarian cancer cell lines. Hey or SKOV3 ovarian cancer cells were infected with either Baco-1 or Synco-2D. Photos were taken at 48 h after initial viral infection (original magnification, ×200). Black arrows indicate a single syncytia formation (FIG. 12E, FIG. 12F).

FIGS. 13A and 13B provide a comparison of the in vitro cytotoxicity of Baco-1 and Synco-2D on ovarian cancer cells. Hey or SKOV3 ovarian cancer cells were seeded into 24-well plates and infected with Baco-1 or Synco-2D, or left uninfected (not shown in this figure). Cells collected 24 h or 48 h after infection, and viable cells were counted after trypan blue staining. The percentage of cell viability was determined by dividing the number of viable infected cells by the number of uninfected cells. Data are expressed as mean±standard deviation of the mean. FIG. 13A shows 0.01 pfu/cell; FIG. 13B shows 0.1 pfu/cell.

FIGS. 14A-14C demonstrate a therapeutic effect of the fusogenic oncolytic HSV for an orthotopic ovarian cancer model. After 14 and 28 days of the orthotopic transplantation of Hey tumor cells, mice received intraperitoneal administration of oncolytic HSV at a dose of 2×10⁷ pfu/200 μl from a different site of tumor injected. The treated group was as follows; (FIG. 14A): PBS as control; (FIG. 14B): Baco-1; (FIG. 14C): Synco-2D. Forty-two days after the orthotopic tumor inoculation, the live mice were euthanized and examined whether they developed tumors and had peritoneal dissemination. Black arrows indicate the formation of peritoneal thickness or dissemination.

FIG. 15 demonstrates survival of mice treated with oncolytic HSV for advanced ovarian cancer (Kaplan-Meier plots). The mice bearing intraperitoneal dissemination of Hey ovarian cancer cells were treated with an intraperitoneal administration of oncolytic HSV as follows; PBS (n=8, ▴), Baco-1 (n=8, ▪), and Synco-2d (n=8, ●). *: A significant prolonging of survival is noted after intraperitoneal administration of Synco-2D versus PBS or Baco-1 (p<0.01).

FIG. 16 provides phenotypic characterization of fusogenic oncolytic HSV in prostate cancer cell line PC-3M-Pro4. PC-3M-Pro4 cells were infected with either non-fusogenic (Baco-1) or fusogenic (Synco-2 or Synco-2D) oncolytic HSVs. Each photo was taken at 24 h or 48 h after initial viral infection (original magnification, ×200). Black arrows indicate the boundary of the syncytium.

FIGS. 17A and 17B show comparison of the in vitro cytotoxicity of Baco-1, Synco-2 and Synco-2D on prostate cancer cells. PC-3M-Pro4 prostate cancer cells were seeded into 24-well plates and infected with Baco-1, Synco-2 or Synco-2D at 0.01 pfu/cell (FIG. 17A) or 0.1 pfu/cell (FIG. 17B), or left uninfected. Cells were collected 24 h, 48 h or 72 h after infection, and viable cells were counted after trypan blue staining. The percentage of cell viability was determined by dividing the number of viable cells from the infected wells by the number of cells in the uninfected well. Data are expressed as mean±standard deviation of the mean. [symbol] , P<0.05 as compared with Baco-1; *, P<0.01 as compared with Synco-2.

FIGS. 18A and 18B show a therapeutic effect of the fusogenic oncolytic HSVs on the orthotopic prostate tumor. Human prostate cancer xenografts were established in the primary site through orthotopic inoculation of PC-3M-Pro4 cells. Eight and 15 days after tumor cell inoculation, mice received intravenous administration of oncolytic HSV at a dose of 2×10⁷ pfu at a volume of 100 μl through tail vein. Forty days after the orthotopic tumor inoculation, mice that were still alive were euthanized and examined for the presence of tumor mass in the original injection site and lymph node metastases. FIG. 18A provides photos that were taken from one mouse from each group that shows an average tumor and local lymph node metastasis (PBS treated group only). The orthotopic tumors are indicated with dashed triangles and the lymph node metastasis is indicated with filled arrows. FIG. 18B shows orthotopic tumors that were explanted and weighed. The plotted figures represent the average weight±standard deviation.

FIGS. 19A through 19C demonstrate that an enhanced oncolytic effect of Synco-2D on 4T1 tumor is accompanied by elevated tumor-specific CTL activities. In FIG. 19A, antitumor activity of oncolytic HSVs on local tumor is shown. The tumor volume was determined by the formula (L×W2)/2, where the L is the tumor length and W the width. In FIG. 19B, a therapeutic effect of oncolytic HSVs on lung metastases is shown. The figure demonstrates the gross pathological findings in a representative mouse from each group. In FIG. 19C, tumor-specific CTL activity after tumor destruction by oncolytic HSVs is provided.

### DETAILED DESCRIPTION OF THE INVENTION

I. Definitions

The term “a” or “an” as used herein the specification may mean one or more. As used herein in the claim(s), when used in conjunction with the word “comprising”, the words “a” or “an” may mean one or more than one. As used herein “another” may mean at least a second or more.

The term “cell membrane fusion” as used herein refers to fusion of an outer membrane of at least two cells, such as two adjacent cells.

The term “conditionally replicating” as used herein refers to the property that a virus can only replicate in, for example, dividing cells (such as tumor cells) but not in, for example, postmitotic or non-dividing cells such as normal hepatocytes or neurons.

The terms “enhanced tumor antigen presentation” or “increased tumor antigen presentation” as used herein refers to an enhancement, increase, intensification, augmentation, amplification, proliferation, multiplication, or combination thereof of the presentation of tumor antigens to the immune system. In a specific embodiment, the presentation comprises the release of tumor antigens. In a specific embodiment, the enhanced tumor antigen presentation is particularly useful for solid tumors, non-solid tumors, and/or metastasized cancer. In a specific embodiment, some exemplary tumor antigens include gp100 and carcinoembryonic antigen (CEA).

The term “improved antitumor immunity” as used herein refers to the generation of a better antitumor immunity in the presence of membrane fusion, (wherein the fusion leads to syncytia formation and enhanced tumor antigen presentation) compared to in the absence of the membrane fusion. In a specific embodiment, the improved antitumor immunity is directed to cell-mediated antitumor immunity.

The term “oncolytic” as used herein refers to an agent that can destroy malignant cells. In a specific embodiment, the destruction comprises fusion of the malignant cell membrane to another membrane. In other embodiments, the destruction comprises lysis of the cell, and in some embodiments the destruction comprises both membrane fusion and lysis.

The term “replication selective” or “replication conditional” as used herein refers to the ability of an oncolytic virus to selectively grow in certain tissues (e.g., tumors).

The term “strictly late viral promoter” as used herein regards a promoter that is only active at a very late stage, i.e., after the start of viral DNA replication.

A skilled artisan recognizes that the term “syncytium” as used herein refers to a multinucleate giant cell formation involving significantly larger number of fused cells.

II. The Present Invention

The present invention regards a conditionally replicating (oncolytic) HSV vector having greater than one cell membrane fusion-generating mechanism, in contrast to those in the related art. In specific embodiments, a fusogenic oncolytic HSV, such as created by methods described herein, for example random mutagenesis, further comprises a nucleic acid encoding a cell membrane fusion-generating polypeptide, such as GALV.fus. In a specific embodiment, the GALV.fus sequence is SEQ ID NO:5.

Oncolytic viruses have shown great promise as anti-tumor agents for solid tumors. However, their anti-tumor potency must be further improved before a clear clinical benefit can be obtained from their administration. The present invention utilizes, in specific embodiments, a gene encoding a truncated form of gibbon ape leukemia virus envelope fusogenic membrane glycoprotein (GALV.fus) into an oncolytic herpes simplex virus through an enforced ligation procedure. In vivo studies show that expression of GALV.fus in the context of an oncolytic virus significantly enhances the anti-tumoral effect of the virus. Furthermore, controlling GALV.fus expression through a strictly late viral promoter, whose activity entirely depends on the initiation of viral DNA replication, led to a strong GALV.fus expression only to tumor tissues. These results demonstrate that the functional expression of a strong fusogenic gene in the context of an oncolytic HSV can enhance the viral anti-tumoral activity without sacrificing the safety of the original virus.

Furthermore, the confinement of transgene expression to tumor cells is particularly desirable for gene therapy of malignant diseases. Current approaches for transcriptional targeting to tumors mainly use tissue-specific promoters to control gene expression. However, these promoters generally have much lower activity than the constitutive viral promoters and may also lose their tissue specificity once cloned into viral vectors. As an alternative approach, the present invention, in some embodiments, utilizes a strict late viral promoter (UL38p), whose activity depends on the onset of viral DNA replication. The promoter was introduced into an oncolytic herpes simplex virus (HSV). In vitro and in vivo characterization showed that in normal non-dividing cells, where the oncolytic HSV has limited ability to replicate, the UL38p has minimal activity. In tumor or cycling cells where the virus can fully replicate, transgene expression from UL38p is almost as high as from the cytomegalovirus immediate early promoter. These results suggest that with an oncolytic virus as a gene delivery vehicle, therapeutic genes may be strongly and specifically expressed by strict late viral promoters such as UL38p in tumors where the oncolytic virus can conditionally replicate.

Thus, in one aspect of the present invention there is a recombinant nucleic acid vector for treatment of a malignant disease in a mammalian patient, wherein the vector comprises a sequence directing the expression on a eukaryotic cell surface of a cell membrane fusion-inducing polypeptide. In other embodiments, the UL38p is utilized to direct expression of the cell membrane fusion-inducing polypeptide.

In some embodiments, the HSV comprises a mutation that provides to the vector cell fusogenic properties. The mutation may be generated randomly, and a pool of potential candidates for having cell fusogenic properties is then assayed for the function by means described herein and/or known in the art. A HSV comprising a mutation may also be obtained from nature, and the corresponding HSV isolated. In specific embodiments, a mutation(s) that renders a HSV as having cell fusogenic and/or syncytial-forming properties is located at or near the glycoprotein B (gB), the gK gene region, or both. The mutation may be a point mutation, a frameshift, an inversion, a deletion, a splicing error mutation, a post-transcriptional processing mutation, a combination thereof, and so forth. The mutation may be identified by sequencing the particular oncolytic HSV, such as Synco-2D, and comparing it to a known wild type sequence.

In specific embodiments, the methods and compositions of the present invention are useful for the treatment of malignant cells, such as, for example, to inhibit their spread, decrease or inhibit their replication, to eradicate them, to prevent their generation or proliferation, or a combination thereof. The malignant cells may be from any form of cancer, and they may be from a solid tumor, although other forms are treatable with methods and compositions herein. In a particular aspect of the invention, the methods and compositions are utilized to treat lung, liver, prostate, ovarian, breast, brain, pancreatic, testicular, colon, head and neck, melanoma, and other types of malignant cells. The invention is useful for treating malignant cells at any stage of a cancer disease, however, in a particular embodiment the invention is utilized upon metastatic stages of the disease. The invention may be utilized in conjunction with another means of therapy for an individual comprising malignant cells.

Furthermore, although conventional radiation therapy and surgery are both potentially curative treatment modalities for organ-confined cancer, such as prostate cancer, there is little effective treatment for metastatic disease, for example, particularly after androgen deprivation therapy fails in prostate cancer. The replication-conditional (oncolytic) viruses of the present invention comprising more than one cell membrane fusion capabilities are useful for the treatment of solid tumors, such as prostate cancer, and the present invention demonstrates that incorporation of cell membrane fusion capability into an oncolytic HSV significantly increases the antitumor potency of the virus.

Although fusogenic oncolytic HSVs may be constructed by any means, so long as greater than one cell membrane fusion capability is present on the vector, in particular embodiments the capability for the fusogenic oncolytic HSVs was generated by one of three different strategies: 1) screening for syncytial phenotype from a vector, such as a well-established oncolytic HSV after random mutagenesis (such as for the generation of Fu-10); 2) insertion of the gene encoding a fusogenic gene product, such as a hyperfusogenic membrane glycoprotein of gibbon ape leukemia virus (GALV.fus), into the genome of an oncolytic HSV (such as for the generation of Synco-2); and 3) incorporation of both of these two membrane fusion mechanisms into a single oncolytic HSV (such as for the generation of Synco-2D).

In particular Examples provided herein, a mouse model bearing both primary tumor and lung metastasis of human prostate cancer xenografts were treated with compositions described herein in a systemic manner. The results show that Synco-2D is a powerful and potent therapeutic agent on this tumor model and that intravenous administration of this virus led to a significant shrinkage of the primary tumor and a dramatic reduction of tumor nodules in the lung. These results suggest that systemic administration of this potent fusogenic oncolytic HSV is an effective treatment for metastatic cancer, particularly human prostate cancer.

III. Cell Membrane Fusing Polypeptides

The present invention in some embodiments comprises at least a fusogenic portion of a cell membrane fusion-inducing polypeptide, such as a viral FMG. In some embodiments the FMG or functional fragment thereof is present on the HSV composition as a nucleic acid encoding an FMG or functional fragment polypeptide. The polypeptide is preferably capable of inducing cell membrane fusion at substantially neutral pH (such as about pH 6-8).

In particular embodiments, the FMG comprises at least a fusogenic domain from a C-type retrovirus envelope protein, such as MLV (as an example, SEQ ID NO:7) or GALV (as an example, SEQ ID NO:5). A retroviral envelope protein having a deletion of some, most, or all of the cytoplasmic domain is useful, because such manipulation results in hyperfusogenic activity for human cells. Particular modifications are introduced, in some embodiments, into viral membrane glycoproteins to enhance their function to induce cell membrane fusion. For example, truncation of the cytoplasmic domains of a number of retroviral and herpesvirus glycoproteins has been shown to increase their fusion activity, sometimes with a simultaneous reduction in the efficiency with which they are incorporated into virions (Rein et al., 1994; Brody et al., 1994; Mulligan et al., 1992; Pique et al., 1993; Baghian et al, 1993; Gage et al., 1993).

A skilled artisan recognizes that in some embodiments it is desirable to introduce functions into a FMG polypeptide, such as novel binding specificities or protease-dependencies, and thereby target their fusogenic activities to specific cell types that express the targeted receptors.

Some examples of cell membrane fusing polypeptides include measles virus fusion protein (SEQ ID NO:8), the HIV gp160 (SEQ ID NO:9) and SIV gp160 (SEQ ID NO:10) proteins, the retroviral Env protein (SEQ ID NO:11), the Ebola virus Gp (SEQ ID NO:12), and the influenza virus haemagglutinin (SEQ ID NO:13).

IV. Nucleic Acid-Based Expression Systems

The present invention is directed to an HSV vector comprising greater than one fusogenic mechanism. In specific embodiments, the vector comprises some or all of the following components.

A. Vectors

The term “vector” is used to refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

In specific embodiments of the present invention, a strict late viral promoter is utilized, which directs expression only in replicating cells, such as tumor cells. Examples of strict late viral promoters include UL38 (SEQ ID NO:3) and Us11 (SEQ ID NO:4). SEQ ID NO:3 is GTGGGTTGCGGACTTTCTGCGGGGCGGCCCAAATGGCCCTTTAAACGTGT GTATACGGACGCGCCGGGCCAGTCGGCCAACACAACCCACCGGAGCGGTAGCCGCG TTTGGCTGTGGGGTGGGTGGTTCCGCCTTGCGT. SEQ ID NO:4 is CTTTTAAGTAAACATCTGGGTCGCCCGGCCCAACTGGGGCCGGGGGTTGGGTCTGGC TCATCTCGAGAGCCACGGGGGGGAACCACCCTCCGCCCAGAGACTCGGGTGATGGT CGTACCCGGGACTCAACGGGTTACCGGATTACGGGGACTGTCGGTCACGGTCCCGC CGGTTCTTCGATGTGCCACACCCAAGGATGCGTTGGGGGCGATTTCGGGCAGCAGCC CGGGAGAGCGCAGCAGGGGACGCTCCGGGTCGTGCACGGCGGTTCTGGCCGCCTCC CGGTCCTCACGCCCCCTTTTATTG.

A promoter generally comprises a sequence that functions to position the start site for RNA synthesis. The best-known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Although the methods and compositions of the present invention preferably utilize strict late viral promoters, for alternative embodiments Tables 1 and 2 list non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin | Banerji et al., 1983; Gilles et al., 1983; Grosschedl |
| Heavy Chain | et al., 1985; Atchinson et al., 1986, 1987; Imler et |
| | al., 1987; Weinberger et al., 1984; Kiledjian et al., |
| | 1988; Porton et at.; 1990 |
| Immunoglobulin | Queen et al., 1983; Picard et al., 1984 |
| Light Chain | |
| T-Cell Receptor | Luria et al., 1987; Winoto et al., 1989; Redondo et |
| | al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 | Greene et al., 1989; Lin et al., 1990 |
| Receptor | |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick et al., 1989; Johnson et |
| Kinase (MCK) | al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell | Hirsch et al., 1990 |
| Adhesion Molecule | |
| (NCAM) | |
| α₁-Antitrypsin | Latimer et al., 1990 |
| H2B (TH2B) | Hwang et al., 1990 |
| Histone | |
| Mouse and/or Type | Ripe et al., 1989 |
| I Collagen | |
| Glucose-Regulated | Chang et al., 1989 |
| Proteins (GRP94 | |
| and GRP78) | |
| Rat Growth | Larsen et al., 1986 |
| Hormone | |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; Sleigh et |
| | al., 1985; Firak et al., 1986; Herr et al., 1986; Imbra |
| | et al., 1986; Kadesch et al., 1986; Wang et al., |
| | 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et at, 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immuno- | Muesing et al., 1987; Hauber et al., 1988; |
| deficiency Virus | Jakobovits et al., 1988; Feng et al., 1988; Takebe et |
| | al., 1988; Rosen et al., 1988; Berkhout et al., 1989 |
| | Laspia et al., 1989; Sharp et al., 1989; Braddock et |
| | al., 1989 |
| Cytomegalovirus | Weber et al., 1984; Boshart et al., 1985; Foecking et |
| (CMV) | al., 1986 |
| Gibbon Ape | Holbrook et al., 1987; Quinn et al., 1989 |
| Leukemia Virus | |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; Searle |
| | | et al., 1985; Stuart et al., |
| | | 1985; Imagawa et al., 1987, |
| | | Karin et al., 1987; Angel et |
| | | al., 1987b; McNeall et al., |
| | | 1989 |
| MMTV (mouse | Glucocorticoids | Huang et al., 1981; Lee et al., |
| mammary tumor | | 1981; Majors et al., 1983; |
| virus) | | Chandler et al., 1983; Lee et |
| | | al., 1984; Ponta et al., 1985; |
| | | Sakai et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, | Hug et al., 1988 |
| | Newcastle Disease | |
| | Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | E1A, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), and human platelet endothelial cell adhesion molecule-1 (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. RES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Vector Delivery and Cell Transformation

In specific embodiments of the present invention, a vector is propagated from the initially infected cell to surrounding cells.

The vector is introduced to the initially infected cell by suitable methods. Such methods for nucleic acid delivery for transformation of an organelle, a cell, a tissue or an organism for use with the current invention are believed to include virtually any method by which a nucleic acid (e.g., HSV vector) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraleyet al., 1979; Nicolauet al., 1987; Wonget al., 1980; Kanedaet al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed. The composition may also be delivered to a cell by administering it systemically, such as intravenously, in a pharmaceutically acceptable excipient to a mammal comprising the cell.

1. Ex Vivo Transformation

Methods for tranfecting vascular cells and tissues removed from an organism in an ex vivo setting are known to those of skill in the art. For example, cannine endothelial cells have been genetically altered by retrovial gene tranfer in vitro and transplanted into a canine (Wilson et al., 1989). In another example, yucatan minipig endothelial cells were tranfected by retrovirus in vitro and transplated into an artery using a double-ballonw catheter (Nabel et al., 1989). Thus, it is contemplated that cells or tissues may be removed and tranfected ex vivo using the nucleic acids of the present invention. In particular aspects, the transplanted cells or tissues may be placed into an organism. In preferred facets, a nucleic acid is expressed in the transplated cells or tissues.

2. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of cell membrane fusion-generating HSV used may vary upon the nature of the, cell, tissue or organism affected.

3. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa el al., 1986) in this manner.

4. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

5. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

6. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

7. Liposome-Mediated Transfection

In a further embodiment of the invention, a nucleic acid, such as an oncolytic HSV, may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinatin virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

8. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al, 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

9. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a cell membrane fusion-generating HSV. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art.

Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

D. Expression Systems

Expression systems may be utilized in the generation of compositions of the present invention. Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. No. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'s COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

V. Mutagenesis

In specific embodiments of the present invention, a vector, such as a non-fusogenic conditionally replicating oncolytic HSV, is mutagenized to generate a fusogenic HSV vector. Where employed, mutagenesis is accomplished by a variety of standard mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations that involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

A. Random Mutagenesis

1. Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et al. 1991). Insertion mutagenesis has been very successful in bacteria and Drosophila (Cooley et al. 1988) and recently has become a powerful tool in corn (Schmidt et al. 1987); Arabidopsis; (Marks et al., 1991; Koncz et al. 1990); and Antirrhinum (Sommer et al. 1990).

Transposable genetic elements are DNA sequences that can move (transpose) from one place to another in the genome of a cell. The first transposable elements to be recognized were the Activator/Dissociation elements of Zea mays (McClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini these terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element. elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

2. Chemical Mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

3. Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

4. In Vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

In recent years, techniques for estimating the equilibrium constant for ligand binding using minuscule amounts of protein have been developed (Blackburn et al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

5. Random Mutagenesis by Fragmentation and Reassembly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

B. Site-Directed Mutagenesis

Structure-guided site-specific mutagenesis represents a powerful tool for the dissection and engineering of protein-ligand interactions (Wells, 1996, Braisted et al., 1996). The technique provides for the preparattion and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

VI. Combination Treatments

In order to increase the effectiveness of the methods and compositions of the present invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that gene therapy could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with the present invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a secondary gene therapy in which a second therapeutic polynucleotide is administered before, after, or at the same time a first therapeutic polynucleotide encoding all of part of a therapeutic polypeptide. Delivery of a vector encoding either a full length or truncated therapeutic polypeptide in conjuction with a second vector encoding one of the following gene products will have a combined anti-hyperproliferative effect on target tissues. Alternatively, a single vector encoding both genes may be used. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

Examples of therapeutic genes that may be utilized in the present invention are included in Table 3.

TABLE 3
Oncogenes

| | | | |
|---|---|---|---|
| Gene | Source | Human Disease | Function |

Growth Factors¹

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma | PDGF B |
| | virus | |

Receptor Tyrosine Kinases^{1,2}

| | | | |
|---|---|---|---|
| ERBB/HER | Avian erythro- | Amplified, | EGF/TGF-α/ |
| | blastosis virus; | deleted squamous | amphiregulin/ |
| | ALV promoter | cell cancer; | hetacellulin |
| | insertion; | glioblastoma | receptor |
| | amplified human | | |
| | tumors | | |
| ERBB-2/ | Transfected from | Amplified breast, | Regulated by NDF/ |
| NEU/HER-2 | rat Glioblatoms | ovarian, gastric | heregulin and EGF- |
| | | cancers | related factors |
| FMS | SM feline | | CSF-1 receptor |
| | sarcoma virus | | |
| KIT | HZ feline | | MGF/Steel receptor |
| | sarcoma virus | | hematopoieis |
| TRK | Transfection from | | NGF (nerve growth |
| | human colon | | factor) receptor |
| | cancer | | |
| MET | Transfection from | | Scatter factor/HGF |
| | human osteo- | | receptor |
| | sarcoma | | |
| RET | Translocations | Sporadic thyroid | Orphan receptor Tyr |
| | and point | cancer; familial | kinase |
| | mutations | medullary thyroid | |
| | | cancer; multiple | |
| | | endocrine | |
| | | neoplasias 2A | |
| | | and 2B | |
| ROS | URII avian | | Orphan receptor |
| | sarcoma Virus | | Tyr kinase |
| PDGF | Translocation | Chronic | TEL (ETS-like |
| receptor | | myclomonocytic | transcription factor)/ |
| | | leukemia | PDGF receptor gene |
| | | | fusion |
| TGF-β | | Colon carcinoma | |
| receptor | | mismatch | |
| | | mutation target | |

NONRECEPTOR TYROSINE KINASES¹

| | | | |
|---|---|---|---|
| ABI. | Abelson Mul.V | Chronic | Interact with RB, |
| | | myelogenous | RNA polymerase, |
| | | leukemia | CRK, CBL |
| | | translocation | |
| | | with BCR | |
| FPS/FES | Avian Fujinami | | |
| | SV; GA FeSV | | |
| LCK | Mul.V (murine | | Src family; T cell |
| | leukemia virus) | | signaling; interacts |
| | promoter | | CD4/CD8 T cells |
| | insertion | | |
| SRC | Avian Rous | | Membrane- |
| | sarcoma Virus | | associated Tyr |
| | | | kinase with |
| | | | signaling function; |
| | | | activated by |
| | | | receptor kinases |
| YES | Avian Y73 virus | | Src family; |
| | | | signaling |

SER/THR PROTEIN KINASES¹

| | | |
|---|---|---|
| AKT | AKT8 murine | Regulated by |
| | retrovirus | PI(3)K?; regulate |
| | | 70-kd S6 k? |
| MOS | Maloney murine | GVBD; cystostatic |
| | SV | factor; MAP kinase |
| | | kinase |
| PIM-1 | Promoter | |
| | insertion Mouse | |
| RAF/MIL | 3611 murine SV; | Signaling in RAS |
| | MH2 avian SV | pathway |

MISCELLANEOUS CELL SURFACE¹

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | Suppressor | | homotypic binding; |
| | | | intracellular |
| | | | interacts with |
| | | | catenins |
| PTC/NBCCS | Tumor suppressor | Nevoid basal cell | 12 transmembrane |
| | and Drosophilia | cancer syndrome | domain; signals |
| | homology | (Gorline | through Gli |
| | | syndrome) | homogue CI to |
| | | | antagonize |
| | | | hedgehog pathway |
| TAN-1 | Notch | T-ALI. | Signaling? |
| homologue | Translocation | | |

MISCELLANEOUS SIGNALING^{1,3}

| | | | |
|---|---|---|---|
| BCL-2 | Translocation | B-cell lymphoma | Apoptosis |
| CBL | Mu Cas NS-1 V | | Tyrosine- |
| | | | phosphorylated |
| | | | RING finger |
| | | | interact Ab1 |
| CRK | CT1010 ASV | | Adapted SH2/SH3 |
| | | | interact Ab1 |
| DPC4 | Tumor suppressor | Pancreatic cancer | TGF-β-related |
| | | | signaling pathway |
| MAS | Transfection and | | Possible angiotensin |
| | Tumorigenicity | | receptor |
| NCK | | | Adaptor SH2/SH3 |

GUANINE NUCLEOTIDE
EXCHANGERS AND BINDING PROTEINS^{3,4}

| | | | |
|---|---|---|---|
| BCR | | Translocated with | Exchanger; protein |
| | | ABL in CML | kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey- | HaRat SV; Ki | Point mutations | Signal cascade |
| Kirsten, | RaSV; Balb- | in many human | |
| N-RAS | MoMuSV; | tumors | |
| | Transfection | | |
| VA V | Transfection | | S112/S113; |
| | | | exchanger |

NUCLEAR PROTEINS AND TRANSCRIPTION FACTORS^{1,5-9}

| | | | |
|---|---|---|---|
| BRCA1 | Heritable | Mammary | Localization |
| | suppressor | cancer/ovarian | unsettled |
| | | cancer | |
| BRCA2 | Heritable | Mammary cancer | Function unknown |
| | suppressor | | |
| ERBA | Avian erythro- | | thyroid hormone |
| | blastosis Virus | | receptor |
| | | | (transcription) |
| ETS | Avian E26 virus | | DNA binding |
| EVII | MuLV promotor | AML | Transcription factor |
| | Insertion | | |
| FOS | FBI/FBR murine | | 1 transcription |
| | osteosarcoma | | factor with c-JUN |
| | viruses | | |
| GLI | Amplified glioma | Glioma | Zinc finger; cubitus |
| | | | interruptus |
| | | | homologue is in |
| | | | hedgehog signaling |
| | | | pathway; inhibitory |
| | | | link PTC and |
| | | | hedgehog |
| HMGG/LIM | Translocation | Lipoma | Gene fusions high |
| | t(3:12) t(12:15) | | mobility group |
| | | | HMGI-C (XT-hook) |
| | | | and transcription |
| | | | factor LIM or acidic |
| | | | domain |
| JUN | ASV-17 | | Transcription factor |
| | | | AP-1 with FOS |
| MLL/ | Translocation/ | Acute myeloid | Gene fusion of |
| VHRX + | fusion ELL with | leukemia | DNA-binding and |
| ELI/MEN | MLL Trithorax- | | methyl transferase |
| | like gene | | MLL with ELI RNA |
| | | | pol II elongation |
| | | | factor |
| MYB | Avian myelo- | | DNA binding |
| | blastosis Virus | | |
| MYC | Avian MC29; | Burkitt's | DNA binding with |
| | Translocation | lymphoma | MAX partner; |
| | B-cell | | cyclin regulation; |
| | Lymphomas; | | interact RB?; |
| | promoter | | regulate apoptosis? |
| | Insertion avian | | |
| | leukosis Virus | | |
| N-MYC | Amplified | Neuroblastoma | |
| L-MYC | | Lung cancer | |
| REL | Avian | | NF-κB family |
| | Retriculoendo- | | transcription factor |
| | theliosis Virus | | |
| SKI | Avian SKV770 | | Transcription factor |
| | Retrovirus | | |
| VHL | Heritable | Von Hippel- | Negative regulator |
| | suppressor | Landau syndrome | or elongin; |
| | | | transcriptional |
| | | | elongation complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary | Ataxia- | Protein/lipid kinase |
| | disorder | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group C | |
| | | (predisposition | |
| | | leukemia | |
| FHIT | Fragile site | Lung carcinoma | Histidine triad- |
| | 3p14.2 | | related diadenosine |
| | | | 5′,3″″-P¹.p⁴ |
| | | | tetraphosphate |
| | | | asymmetric |
| | | | hydrolase |
| hMLI/MutL | | HNPCC | Mismatch repair; |
| | | | MutL homologue |
| hMSH2/ | | HNPCC | Mismatch repair; |
| MutS | | | MutS homologue |
| hPMS1 | | HNPCC | Mismatch repair; |
| | | | MutL homologue |
| hPMS2 | | HNPCC | Mismatch repair; |
| | | | MutL homologue |
| INK4/MTS1 | Adjacent INK-4B | Candidate MTS1 | p16 CDK inhibitor |
| | at 9p21; CDK | suppressor and | |
| | complexes | MLM melanoma | |
| | | gene | |
| INK4B/ | | Candidate | p15 CDK inhibitor |
| MTS2 | | suppressor | |
| MDM-2 | Amplified | Sarcoma | Negative regulator |
| | | | p53 |
| p53 | Association with | Mutated >50% | Transcription factor; |
| | SV40 T antigen | human tumors, | checkpoint control; |
| | | including | apoptosis |
| | | hereditary | |
| | | Li-Fraumeni | |
| | | syndrome | |
| PRAD1/ | Translocation | Parathyroid | Cyclin D |
| BCL1 | with Parathyroid | adenoma; B-CLL. | |
| | hormone or IgG | | |
| RB | Hereditary | Retinoblastoma; | Interact cyclin/cdk; |
| | Retinoblastoma; | osteosarcoma; | regulate E2F |
| | Association with | breast cancer; | transcription factor |
| | many DNA virus | other sporadic | |
| | tumor Antigens | cancers | |
| XPA | | xeroderma | Excision repair; |
| | | pigmentosum; | photo-product |
| | | skin cancer | recognition; zinc |
| | | predisposition | finger |

Although a skilled artisan would know which therapeutic sequences to obtain for a therapy, as well as their identity from a database such as the National Center for Biotechnology Information's GenBank database, an exemplary sequence includes p53 (M14695; SEQ ID NO:6) for cancer treatment.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VII. Pharmaceutical Compositions and Routes of Administration

Compositions of the present invention will have an effective amount of a nucleotide sequence for therapeutic administration in combination and, in some embodiments, is combined with an effective amount of a compound (second agent) that is therapeutic for the respective appropriate disease or medical condition. Such compositions will generally be dissolved or dispersed in a pharmaceutically acceptable carrier or aqueous medium. The term “effective” or “therapeutically effective” as used herein refers to inhibiting an exacerbation in symptoms, preventing onset of a disease, amelioration of at least one symptom, or a combination thereof, and so forth.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-disease agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The compositions of the present invention may advantageously be administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for therapy may be assembled together in a kit. When the components of the kit are provided in one or more liquid solutions, the liquid solution preferably is an aqueous solution, with a sterile aqueous solution being particularly preferred.

For in vivo use, an agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In one exemplary method wherein the therapy is for an individual with cancer, targeting of cancerous tissues may be accomplished in any one of a variety of ways. For example, a composition of the present invention, such as an AE-DNA with an appropriate homologous exchangeable piece may be complexed with liposomes and injected into patients with cancer; intravenous injection can be used to direct the gene to all cell. Directly injecting the liposome complex into the proximity of a cancer can also provide for targeting of the complex with some forms of cancer. For example, cancers of the ovary can be targeted by injecting the liposome mixture directly into the peritoneal cavity of patients with ovarian cancer. Of course, the potential for liposomes that are selectively taken up by a population of cancerous cells exists, and such liposomes will also be useful for targeting the gene.

Those of skill in the art will recognize that the best treatment regimens for using a composition of the present invention to provide therapy can be straightforwardly determined. This is not a question of experimentation, but rather one of optimization, which is routinely conducted in the medical arts. For example, in vivo studies in nude mice provide a starting point from which to begin to optimize the dosage and delivery regimes. The frequency of injection will initially be once a wk, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of composition used in mice. In certain embodiments it is envisioned that the dosage may vary from between about 1 mg composition DNA/Kg body weight to about 5000 mg composition DNA/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg composition DNA/Kg body to about 20 mg composition DNA/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

VIII. Delivery of Nucleic Acid Molecules

Several non-viral gene delivery vectors for the transfer of a polynucleotide(s) of the present invention into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the polynucleotide has been delivered into the cell the sequence of the AE-DNA homologous to the target DNA is positioned accordingly. In certain embodiments, the polynucleotide may be stably integrated into the genome of the cell by methods described herein. How the polynucleotide is delivered to a cell and where in the cell the nucleic acid remains is dependent on a number of factors known in the art.

In yet another embodiment of the invention, the expression vector may simply consist of naked recombinant DNA or plasmids comprising the polynucleotide. Transfer of the construct may be performed by any of the methods mentioned above which physically or chemically permeabilize the cell membrane. This is particularly applicable for transfer in vitro, but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transfer of a naked DNA into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, a polynucleotide may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinatin virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other polynucleotides that can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells.

In certain embodiments, DNA transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods for Examples 2-5

Cell Lines

African green monkey kidney (Vero) cells, human embryonic fibroblasts (HF 333.We), human tumor cell lines U-87 MG (glioblastoma), Hep 3B (hepatocellular carcinoma) and human prostate cancer line DU 145 were obtained from American Tissue Culture Collection (Rockville, Md.). All the cells were cultured with DMEM containing 10% fetal bovine serum (FBS).

Generation of Fusogenic Oncolytic HSVs

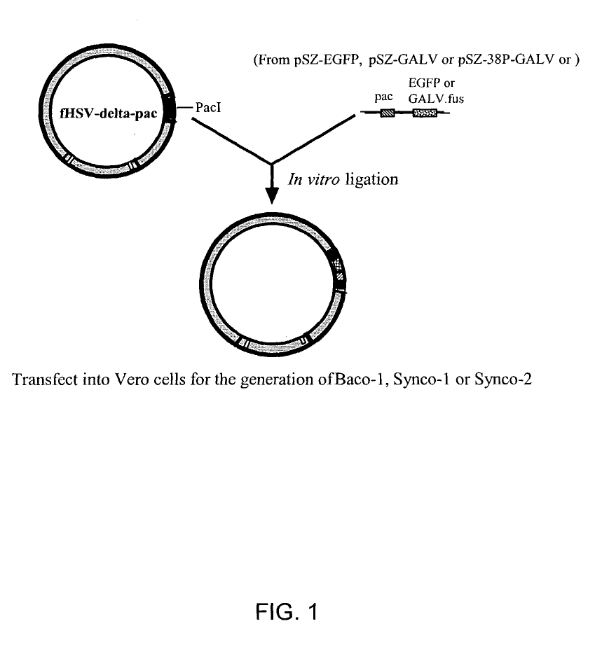

All of the oncolytic HSVs were derived from fHSV-delta-pac, a bacterial artificial chromosome (BAC) construct containing a mutant form of HSV-1 genome (with deletion of γ34.5 genes and HSV packaging signals) (Saeki et al., 1998). Infectious HSV were generated from fHSV-delta-pac by a 2-step procedure (FIG. 1). First, gene cassettes including enhanced green fluorescent protein (EGFP) and GALV-fus were first cloned into pSZ-pac, which contains a copy of the HSV pac (an exemplary sequence being SEQ ID NO:14) and a cytomegalovirus immediate early promoter. The EGFP gene cassette was cut out from pEGFP-N1 (Clontech) with AseI and AflIII and cloned into pSZ-pac, to create pSZ-EGFP. The GALV.fus gene was cut out from pCR3.1-GALV with NheI and NotI and cloned into pSZ-pac, to generate pSZ-GALV. The promoter and the enhancer region (das) of UL38 have been well defined (Guzowski and Wagner, 1993). The promoter was amplified (including the das sequence) from HSV-1 genome with this pair of oligos: forward primer 5′ GTGGGTTGCGGACTTTCTGC 3′ (SEQ ID NO:1), and reverse primer 5′ ACACTCACGCAAGGCGGAAC 3′ (SEQ ID NO:2). The PCR product was cloned into pSZ-pac to replace the CMV promoter, to generate plox-UL38P. The GALV.fus gene obtained from NheI and NotI digestion was then cloned into plox-UL38P so that the gene is driven by the UL38 promoter, to generate pSZ-38P-GALV.

Infectious HSV was obtained from fHSV-delta-pac, the GALV.fus or EGFP, together with the HSV pac, by cutting out from pSZ-EGFP based plasmids (pSZ-EGFP, pSZ-GALV, pSZ-38P-GALV) and direct ligation into the unique PacI site of fHSV-delta-pac. The DNA ligation mixture was transfected into Vero cells using Lipofectamine (Gibco-BRL) and incubated for 3-5 days for the infectious virus to be generated. The viruses, designated Baco-1 (containing the DNA fragment of EGFP and pac), Synco-1 (containing the DNA fragment of CMV-GALV.fus and pac) and Synco-2 (containing UL38P-GALV.fus and pac), were subsequently plaque purified. Virus stocks were prepared by infecting Vero cells with the viruses at 0.01 plaque-forming units (pfu) per cell, and were harvested after 2 days and stored at −80° C.

Phenotypic Characterization of Fusogenic Oncolytic HSVs

Cells were seeded into six-well plates and infected the following day with each virus at a dose ranging from 0.1 to 0.0001 pfu/cell. Cells were cultured in a maintenance medium (containing 1% FBS) and were left for up to two days to allow the fusion pattern and plaques to develop. To block HSV replication, acyclovir was added into the culture medium at a final concentration of 100 μM. To slow down the division or arrest the embryonic fibroblasts, cells were either starved for FBS for 48 h, or starved for FBS and incubated with 20 μM lovastatin at the same time for 24 h, before the cells were infected with the viruses. Lovastatin is a chemical that induces cell-cycle arrest but does not interfere with HSV replication (Schang et al., 1998).

In Vitro Cell Killing Assay

Each of the three human tumor cell lines were seeded into 48-well plates and were infected with each virus at 0.1 and 0.01 pfu/cell, or were left uninfected. Cells were harvested 24 h and 48 h later through trypsinization. The number of surviving cells was counted on a hemocytometer following trypan blue staining. The percentage of cell survival was calculated by dividing the number of viable cells from the infected well by the number of cells from the well that was left uninfected. The experiments were done in triplicate, and the averaged numbers were used for the final calculation.

Animal Studies

Six-week-old male Hsd athymic (nu/nu) mice were purchased from Harlan (Indianapolis, Ind.). Hep 3B cells were cultured in standard conditions and were harvested in log phase with 0.05% trypsin-EDTA. The cells were washed twice with serum-free medium before they were resuspended in PBS at a concentration of 5×10⁷-cells/ml. A total of 100 μl cell suspension containing 5×10⁶ cells were subcutaneously injected in the right flank of mice. When tumors reached to approximately 5-8 mm in diameter, a single injection of 1×10⁷ pfu of each virus at 100 μl volume was administered intratumorally (n=10). Control tumors received the same amount of PBS only. Tumor size was measured weekly for 4 weeks and the tumor volume was calculated using the formula: tumor volume [mm]³=0.5×(length [mm])×(width [mm])².

For pathological examination of the tumor tissues, mice were sacrificed 5 days after virus or PBS injection. The tumors were removed, fixed and stained with hematoxylin and eosin.

Statistical values are presented as mean±standard deviation of the mean; comparisons were made using student's t-test. Statistical significance was defined asp <0.05.

### Example 2

### Enforced Ligation of GALV.fus Gene into an Oncolytic HSV and In Vitro Characterization of the New Virus

Earlier attempts to clone the GALV.fus gene into an oncolytic adenovirus were not successful (Diaz et al., 2000). Two reasons may have resulted in this failure. First, the severe and rapid syncytial formation after GALV.fus gene transfection might have interfered with the subsequent homologous recombination for the recombinant virus generation. Second, expression of GALV.fus in the context of an adenovirus may interfere with the infection process of the virus. Unlike adenovirus that is non-enveloped, HSV is an enveloped virus whose infection naturally involves membrane fusion. It was therefore anticipated that it was likely that HSV might be able to withstand the membrane-fusion effect from genes such as GALV.fus and continue to grow. To avoid the potential interference from the initial membrane fusion of GALV.fus gene transfection, an enforced direct-ligation strategy for inserting GALV.fus gene into the genome of an oncolytic HSV was utilized (FIG. 1). fHSV-delta-pac is a bacterial artificial chromosome (BAC) based construct that contains a mutated HSV genome, in which the diploid gene encoding r34.5 was partly deleted, and both copies of HSV packaging signal (pac) were completely deleted (Saeki et al., 1998). Therefore, infectious HSV can not be generated from the construct unless an intact HSV pac is provided in cis. Also, any virus eventually generated from this construct will be replication selective, due to the partial deletion of γ34.5 gene. The GALV.fus gene cassette (driven by CMV immediate early promoter) was linked with an intact HSV pac in a plasmid. The GALV.fus-pac sequence was then ligated directly into the unique PacI site located in the BAC sequence of fHSV-delta-pac. The ligation mixture was directly transfected into Vero cells, and the virus grown from the cells was collected and plaque purified. One of the plaque-purified viruses was named Synco-1 and was used subsequently.

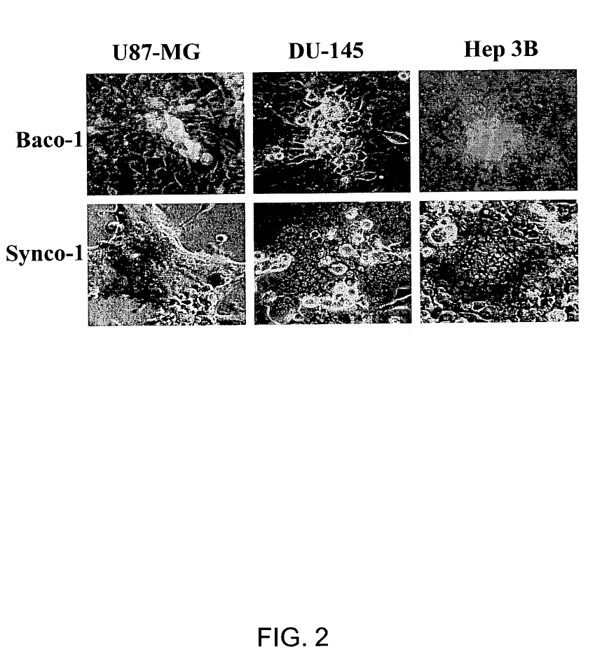

Synco-1 was phenotypically characterized. Human cancer cells of different tissue origins were infected with the virus at a low multiplicity of infection (MOI). At 36 h after infection, a clear syncytial phenotype was observed on all of the three tumor cells tested (FIG. 2, bottom panel). By average, each syncytium covers an area of around 1,000 cells, indicating that a large number of tumor cells were involved in each syncytium. On the other hand, no obvious syncytium was observed in the same tumor cells that were infected with Baco-1 (FIG. 2, top panel), which was also derived from fHSV-delta-pac but contains the EGFP gene instead of GALV.fus. The area covered by each individual plaque from Baco-1 was substantial smaller than the syncytia from Synco-1.

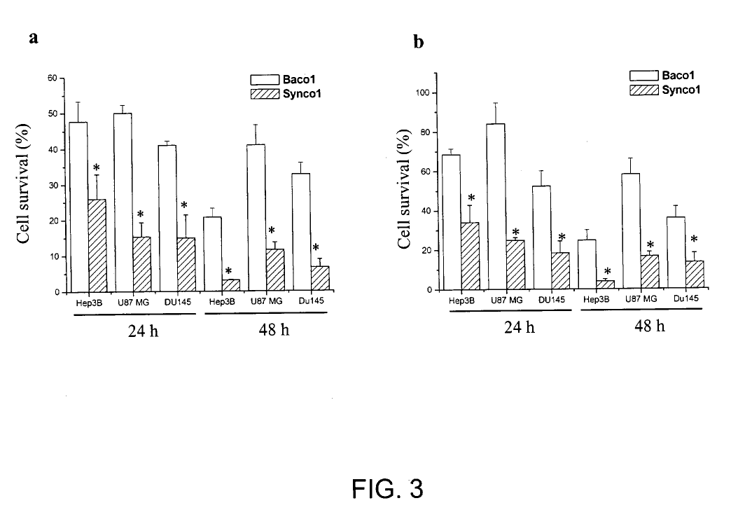

FMG-mediated syncytial formation in the context of oncolytic HSV was demonstrated to result in an increased tumor cell killing. The cell viability was compared after the cells were infected with either Baco-1 or Synco-1 at a relatively low MOI (0.1 and 0.01 pfu/cell), so that both the inherent cytotoxicity of the input virus as well as the ability of the virus to replicate and spread in these cells could be assessed. The cytotoxic effect of the virus infection on the tumor cells was quantified by calculating the percentage of cells that survived at different time points after the virus infection. In general, Synco-1 had a significantly stronger ability to kill these tumor cells than Baco-1 at both time points and with both viral doses (FIGS. 3A and 3B).

### Example 3

### Enhanced Anti-Tumor Effect of Synco-1 in Xenografted Human Cancer

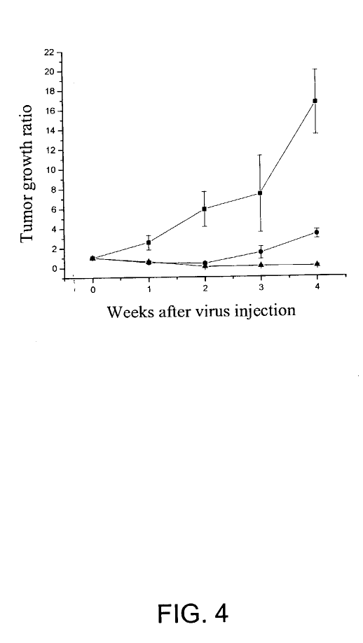

The syncytial phenotype and the enhanced tumor cell killing ability of Synco-1 converted to an enhanced anti-tumor effect in vivo. The virus was injected directly into established xenografts of Hep 3B, with tumor diameters ranging from 5 mm to 8 mm. A single intratumoral injection was given with a dose of 1×10⁷ pfu. This is in contrast to literatures where multiple injections with substantially higher viral dose were usually given (Mineta et al., 1995; Pawlik et al., 2000). The tumor size was measured weekly for 4 weeks. Compared with PBS control, injection of both Baco-1 and Synco-1 had immediate effect on the tumor growth (FIG. 4). Starting from week 1 after virus injection, the tumor size from mice receiving both Baco-1 and Synco-1 was significantly smaller than the tumors injected with PBS (p<0.001). On the other hand, starting from week 2, Synco-1 showed significantly enhanced anti-tumor effect than Baco-1 (P<0.001). Half of the animals (5 out of 10) showed tumor free in week 3 after Synco-1 administration, and the other half also had significantly reduced tumor size. In contrast, only one mouse in Baco-1 injected group showed tumor free. The tumors in other 9 mice shrank initially, then started to re-grow by week 3 after virus injection. By week 4 after virus administration—the end point of the experiment, the tumor sizes in Synco-1 injected group still remained small or tumor free, the average tumor size from Baco-1 injection becomes substantially larger than the tumor size of pre-virus administration.

### Example 4

### Conditional Expression of GALV.fus through a Strictly Late Viral Promoter

Uncontrolled expression of GAVL.fus, even in the context of an oncolytic virus, will undoubtedly pose a safety concern for the use of this virus. This is especially true when systemic administration is required, e.g. for disseminated metastatic tumors. A logical way to overcoming this potential problem is to use a tumor or tissue specific promoter to control the GALV.fus expression. Although there are quite a few tumor or tissue specific promoters reported, they generally have much lower activity than viral promoters and they also intend to lose their tissue specificity once cloned into viral vectors. The HSV genome transcription is a regulated cascade in which early and late phases of gene expression are separated by viral DNA replication. In particular, some of the late transcripts can be catagorized as strict late, whose expression are strictly depending on the initiation of viral DNA replication. It was reasoned that directing GALV.fus expression with such a strictly late viral promoter, in the context of an oncolytic HSV, leads to a high level, selective GALV.fus gene expression in tumor tissues.

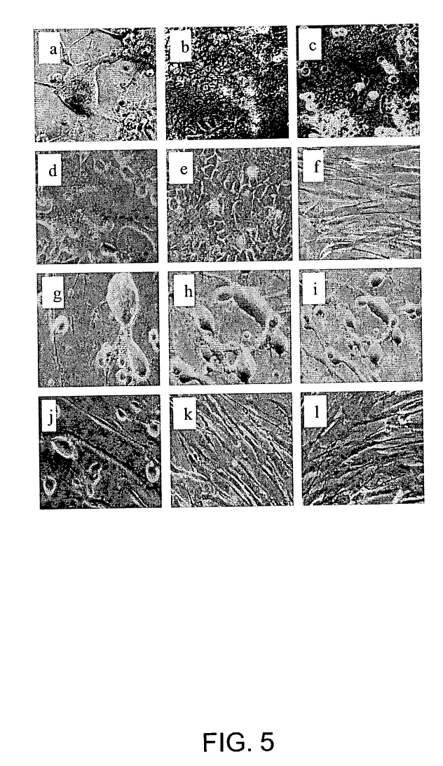

The UL38 promoter is a well-characterized strictly late viral gene and its promoter region has been well defined (Guzowski and Wagner, 1993). The GALV.fus gene was linked with the UL38 promoter and subsequently inserted as the gene cassette into fHSV-delta-pac by the strategy described in FIG. 1, to create Synco-2. Infection of different human tumor cells with Synco-2 showed that this virus also has a clear syncytial phenotype in these cells (FIGS. 5A, 5B, 5C) and the extent of the cell fusion is similar to that of Synco-1 (as shown in FIG. 2).

Two experiments were performed to determine if GALV.fus—mediated syncytial formation in Synco-2 is truly dependent on viral DNA replication. Firstly, human tumor cells were infected with either Synco-1 or Synco-2, with or without the presence of acyclovir (ACV)—a strong inhibitor of HSV DNA replication. The syncytial formation from Synco-1 is largely unaffected by the presence of ACV (FIG. 5D), while cell membrane fusion from Synco-2 was completely blocked by this drug (FIG. 5E). Secondly, it was directly tested if Synco-2 loses its ability of causing syncytial formation in normal non-dividing human cells. Primary human fibroblasts of either quiescent or cycling state were infected with either Synco-1 (FIGS. 5G-I) or Synco-2 (FIGS. 5J-L). The infection from both viruses caused syncytial formation this normal human cells when the cells were kept in dividing status, with the syncytia from Synco-1 infection (FIG. 5G) were slightly stronger than those from Synco-2 infection (FIG. 5J). Synco-1 mediated cell fusion is only slightly affected in the cells whose cycling was either slowed down (FIG. 5H) or completely arrested (FIG. 51). On the other hand, Synco-2 medicated cell fusion was completely blocked in the cells whose cycling was slowed down (FIG. 5K) or arrested (FIG. 5L). The infected cells looked almost identical to the uninfected cells (FIG. 5F). Together, these results demonstrate that UL38 promoter is a strong and tumor specific promoter in the context of an oncolytic HSV and that driving GALV.fus gene with this promoter in an oncolytic HSV will create minimal extra side-effect from the administration of the virus.

### Example 5

### Equivalent Anti-Tumor Potency of Synco-2 and Synco-1

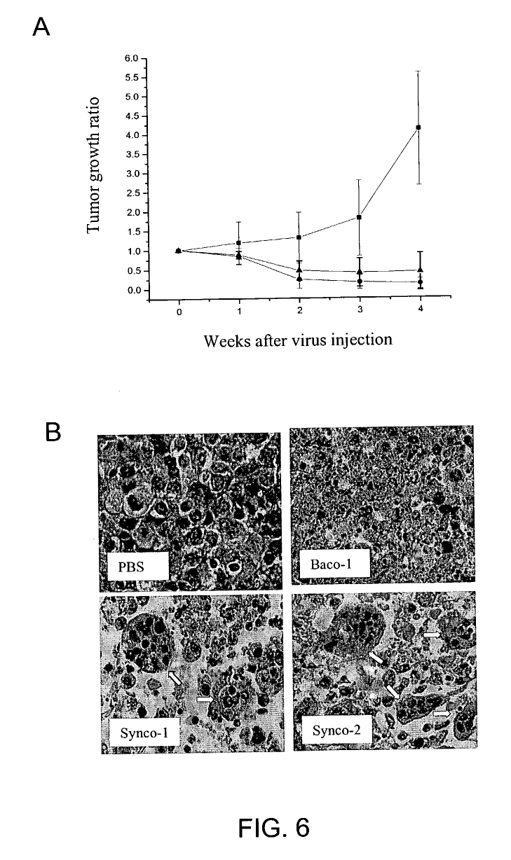

The anti-tumor potency was compared between Synco-1 and Synco-2 by injecting the viruses into xenografted human liver cancer in nude mice through the same therapeutic scheme as described earlier. The results showed that both Synco-1 and Synco-2 have significantly better anti-tumor effect than Baco-1, starting from week 2 after virus administration (p<0.001). On the other hand, there was no significant difference on the anti-tumor effect between Synco-1 and Synco-2 (FIG. 6A). Also administration of either Synco-1 or Synco-2 resulted in the same number of mice tumor-free (5/10). These results demonstrate that Synco-2 posses an equivalent anti-tumor potency as Synco-1, even though the GALV.fus expression in the virus is driven by a conditional viral promoter.

Syncytial formation is indeed part of the oncolytic process of Synco-1 and Synco-2 mediated anti-tumor therapy. Established Hep 3B tumors were injected with oncolytic HSVs or PBS, and the tumors were excised 5 days later. Examination of stained tumor sections revealed that syncytia comprising a varying number of cell nucleus were frequently encountered across the tissue section of tumors injected with either Synco-1 or Synco-2 viruses (FIG. 6B). Such syncytia were not seen in tissue sections of tumors injected with either Baco-1 or PBS.

### Example 6

### Exemplary Methods and Materials for Examples 7-9

Cells

Both Vero (African green monkey kidney fibroblasts) cells, human embryonic fibroblasts (HF 333.We), and Hep 3B (human liver cancer cells) were obtained from the American Type Culture Collection. They were cultured in Dulbecco's modified Eagle medium (DMEM) supplemented with 10% fetal bovine serum (FBS).

Plasmid Constructions

The promoter and the enhancer region (das) of UL38, which has been well defined (Guzowski and Wagner, 1993), was amplified from HSV-1 DNA with the following pair of primers: forward 5′-GTGGGTTGCGGACTTTCTGC-3′ (SEQ ID NO:1) and reverse 5′-ACACTCACGCAAGGCGGAAC-3′ (SEQ ID NO:2). The amplified promoter sequence was cloned into the unique NcoI site of plox-3H, which lies next to a copy of the HSV packaging signal flanked by recognition sites for the restriction enzyme PacI, to generate plox-UL38p. The gene encoding the secreted form of alkaline phosphatase (SEAP) was cut out from pSEAP-control (CLONTECH Laboratories, Inc. Palo Alto, Calif.) with HpaI and HindIII, and cloned into the unique BglII site of plox-UL38p through blunt-end ligation. So in the new plasmid, designated pLox-AP, the SEAP gene is driven by the UL38 promoter. PIMJ-pac-AP was constructed by inserting the same AP gene (HpaI-HindIII fragment of pSEAP-control) into the downstream of the CMV-P contained in pIMJ-pac, which lies next to a copy of HSV pac flanked by PacI recognition sites. Pure plasmid DNA was obtained by alkaline lysis of bacterial culture and purified by QIAGEN-tip 500 column (QIAGEN, Valencia, Calif.).

Generation of Oncolytic HSV Containing AP Gene Cassettes

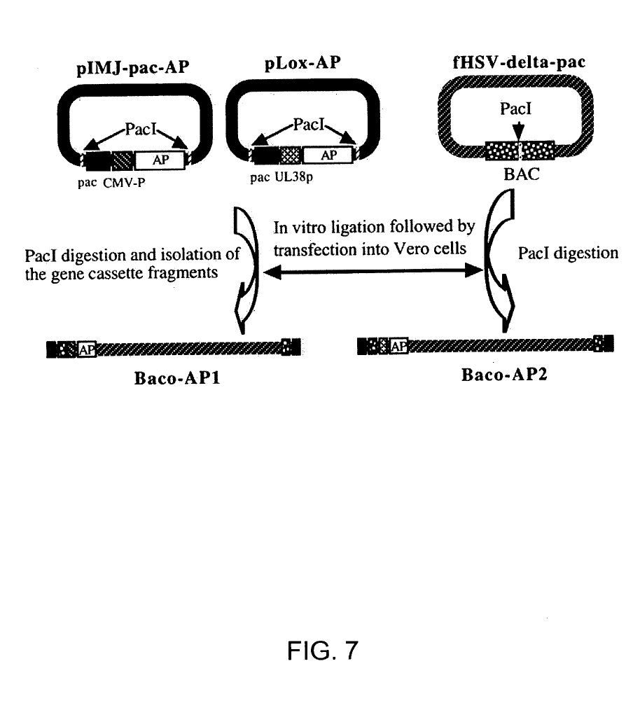

The AP gene cassette together with the pac sequence were cut out from either pLox-AP or pIMJ-pac-AP with PacI and gel-purified. They were then cloned into fHSV-delta-pac for the generation of infectious oncolytic HSV through an enforced ligation strategy. As illustrated in FIG. 7, fHSV-delta-pac is a bacterial artificial chromosome (BAC)-based HSV construct. As the diploid gene encoding γ34.5 and both copies of HSV packaging signal (pac) have been deleted from the HSV sequence contained in fHSV-delta-pac (Saeki et al., 1998), infectious HSV cannot be generated from this construct unless an intact HSV pac is provided in cis, and the generated virus is replication-conditional due to the deletion of both copies of γ34.5 gene. The ligation mixture was directly transfected into Vero cells using Lipofectamine (Gibco-BRL) and incubated for 3-5 days for the infectious virus to be generated. The viruses, designated Baco-API (containing CMV-P-AP cassette) and Baco-AP2 (containing UL38p-AP cassette), were subsequently plaque purified. The presence of AP gene in the viruses was confirmed by detection of AP expression. To generate large viral stocks, Vero cells were infected with each of the viruses at 0.01 plaque-forming unit (pfu) per cell. The viruses were harvested 2 days later and subjected to 3 cycles of freeze-thaw, which was followed by 1 cycle of sonication. Cell debris was removed by low-speed centrifugation (2,000 g at 4° C. for 10 min), and the virus stocks were stored at −80° C.

Transfection and Infection of Mammalian Cells In Vitro for Quantification of AP Release

For in vitro plasmid DNA transfection, Vero cells were seeded in 6-well plates one day earlier at 2×10⁵ cells/well and incubated at 37° C. in a 5% CO₂ atmosphere. The plasmid DNA (2 μg) of pLox-AP or pIMJ-pac-AP was mixed with 5 μl of lipofectamine (GibcoBRL) according to the manufacturer's instruction. Before being applied to cells, the liposome-formulated DNA was added to 1 ml of DMEM without serum. The cells (about 70% confluent) were exposed to the DNA-liposome complex for 3 h at 37° C., after which the transfection mixture was replaced with 2 ml DMEM containing 10% FBS. Sixteen hours after transfection, the cells were either infected with 0.1 pfu/cell of Baco-1, an oncolytic HSV constructed the same way as Baco-AP1 or Baco-AP2 but contains the EGFP gene cassette instead, or mock infected (with medium only). The medium was collected 24 h later for the measurement of AP release.

For in vitro characterization of Baco-API and Baco-AP2, the embryonic fibroblasts were either kept in cycling phase by growing in medium containing 10% FBS, or were arrested with 20 μM lovastatin for 30 h in serum-free medium. Lovastatin is a chemical that induces cell-cycle arrest but does not interfere with HSV replication (Schang et al., 1998). The cells were then infected with either Baco-AP1 or Baco-AP2 at 0.1 pfu/cell and the medium was collected 24 h after infection for the quantification of AP release.

Chemiluminescent AP Assay

AP activity in culture medium or blood serum was quantified using a commercial detection kit from CLONTECH Laboratories, Inc. (Palo Alto, Calif.). The assay was performed according to the manufacturer's instructions. Briefly, 25 μl of sample was added to 75 μl of 1×dilution buffer. After gentle mixing, the diluted samples were incubated at 65° C. for 30 min. The samples were cooled to room temperature, and 100 μl of assay buffer was added to each sample, followed by incubation for 5 min. at room temperature. Finally, samples were mixed with the solution containing chemiluminescent substrate C and enhancer at a ratio of 1:20, and incubated for 30 min at room temperature. The chemiluminescent emission was detected using a luminometer (Turner Designs Instruments; Sunnyvale, Calif.).

Animal Studies

Six-week-old female Hsd athymic (nu/nu) mice were obtained from Harlan (Indianapolis, Ind.). For the establishment of liver cancer, Hep 3B cells were cultured in standard conditions and were harvested in log phase with 0.05% trypsin-EDTA. The cells were washed twice with serum-free medium before they were resuspended in PBS at a concentration of 5×10⁷ cells/ml. A total of 5×10⁶ cells (in a 100-μl suspension) were subcutaneously injected into the right flank of each mouse. For intratumoral injection, when the tumors reached approximately 8 mm in diameter, they were injected with viruses (5×10⁶ pfu in a 100 μl volume). For i.v. (tail-vein) or i.m. (into the right hind limb) injection, mice without established tumor were injected with the same amount of viruses. Blood was withdrawn from the mice at days 1, 2, 3, 4, 5 and 7 after virus inoculation for quantification of AP release.

### Example 7

### Construction and Characterization of Vectors

To facilitate the in vitro and in vivo analysis of gene expression, the SEAP gene was linked with the UL38 promoter (UL38p). The same SEAP gene was also linked to the CMV-P as a control. To clone the gene into the oncolytic HSV, an enforced ligation strategy was utilized, which is more reliable and efficient for cloning foreign genes into HSV genome than the traditional homologous recombination. As depicted in FIG. 7, fHSV-delta-pac is a bacterial artificial chromosome (BAC) based construct that contains a mutated HSV genome, in which the diploid gene encoding γ34.5 was partly deleted, and both copies of HSV packaging signal (pac) were completely deleted (Saeki et al., 1998). Therefore, infectious HSV can not be generated from this construct unless an intact HSV pac is provided in cis. Any virus generated from this construct will be replication selective, due to the partial deletion of both copies of the γ34.5 gene. The SEAP gene cassettes were initially cloned into a middle plasmid to generate pIMJ-pac-AP and pLox-AP in which the gene cassettes are flanked by the recognition sites of restriction endonuclease PacI. The DNA fragments were then cut out with PacI and ligated into the unique PacI site located in the BAC sequence of fHSV-delta-pac. The ligation mixture was directly transfected into Vero cells and the virus grown from the cells was collected and plaque purified, to generate Baco-AP1 (containing CMV-P-SEAP cassette) and Baco-AP2 (containing UL38p-SEAP cassette), respectively (FIG. 7).

The two viruses (Baco-API and Baco-AP2) were then directly compared for their growth property in vitro. Vero cells were infected with the viruses at three different multiplicities of infection: 0.1, 1, and 5 plaque forming units (pfu) per cell. The viruses were harvested 24 and 48 h after infection and titrated by a plaque assay. There was no significant difference in the replication of these two viruses (Table 4).

TABLE 4
Comparison of growth property of Baco-AP1 and Baco-AP2

| | |
|---|---|
| 24 h | 48 h |

| MOI | Baco-AP1 | Baco-AP2 | Baco-AP 1 | Baco-AP2 |
|---|---|---|---|---|
| 0.1 | 5.1 × 10⁵ | 4.2 × 10⁵ | 8.6 × 10⁶ | 7.9 × 10⁶ |
| 1 | 4.4 × 10⁶ | 5.3 × 10⁶ | 3.7 × 10⁶ | 3.4 × 10⁶ |
| 5 | 2.8 × 10⁶ | 3.5 × 10⁶ | 2.2 × 10⁶ | 2.7 × 10⁶ |

### Example 8

### In Vitro Characterization of UL38 Promoter Activity

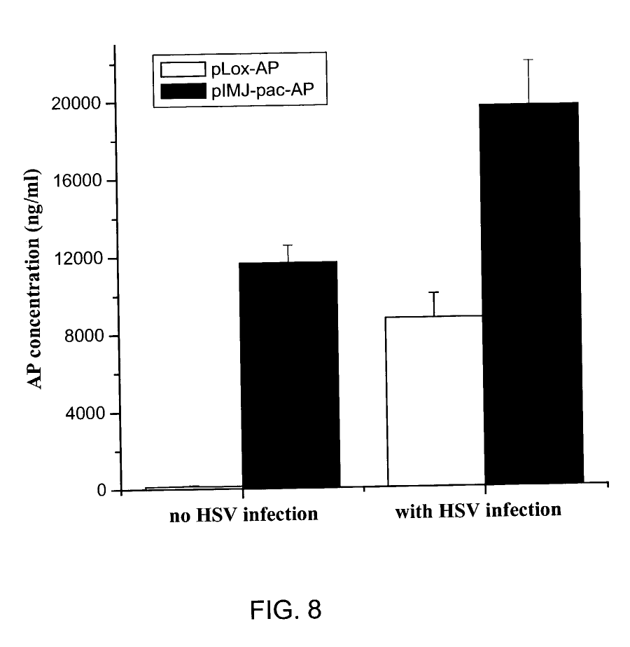

The background level of UL38p activity was determined, and the effect of HSV replication on transactivation of the promoter was investigated. pIMJ-pac-AP (containing CMV-P AP cassette) and pLox-AP (containing UL38p AP cassette) DNA was transfected into Vero cells in duplicates. Sixteen hours after transfection, one set of the transfected cells were infected with an oncolytic HSV (Baco-1), which was constructed the same way as Baco-API or Baco-AP2 except that it contained the enhanced green fluorescent protein (EGFP) gene cassette instead of SEAP. The other set of transfected cells was mock infected (with medium only). Twenty-four hours later, the medium was collected from both sets of cells and the AP released in the medium was quantified. The results showed that without HSV infection, AP in the medium of cells transfected with pLox-AP was barely detectable (FIG. 8). However, in the presence of an oncolytic HSV infection, the AP expression increased by 70 fold, reaching almost half the level of the AP released from pIMJ-pac-AP-transfected cells. HSV infection also substantially increased the level of AP expression from CMV-P, likely because HSV infection transactivates CMV-P activity (Herrlinger et al., 2000). These data demonstrate that the UL38p has extremely low basal activity, which is greatly increased in the presence of lytic HSV infection.

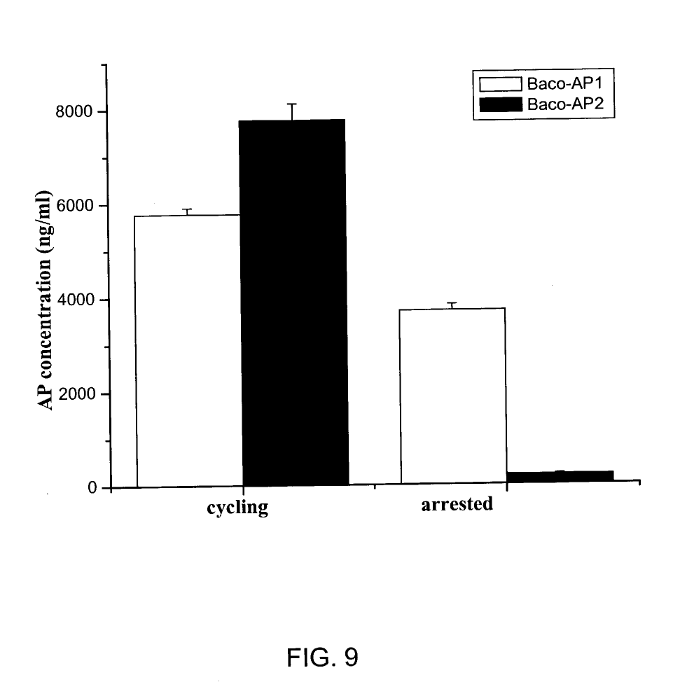

The activity of UL38p in an oncolytic HSV in vitro was characterized. The promoter activity was compared in normal human cells in either a quiescent or a cycling state. Primary human fibroblasts were plated in 12-well plates in duplicate. One set was treated with 20 μM lovastatin, a drug that induces cell-cycle arrest but does not interfere with HSV replication (Schang et al., 1998). Both arrested and un-treated (i.e. cycling) cells were then infected with either Baco-AP1 or Baco-AP2 at 0.1 pfu/cell. The culture medium was collected 24 h after infection and the AP in the medium was quantified. The result showed that the AP expression from UL38p (from Baco-AP2 infected cells) was actually slightly higher than that of CMV-P (from Baco-AP1) in the cycling cells (FIG. 9). However, when the cell cycle was arrested, UL38p activity was reduced to low levels. Due to the inhibitory effect of cell arrest on the replication and spread of oncolytic HSV, the AP expression from the CMV-P is also substantially reduced, but the degree of inhibition in the UL38p containing virus (Baco-AP2) is much more substantial (less than two fold versus 40-fold).

### Example 9

### In Vivo Characterization of UL38 Promoter Activity in the Context of Oncolytic HSV

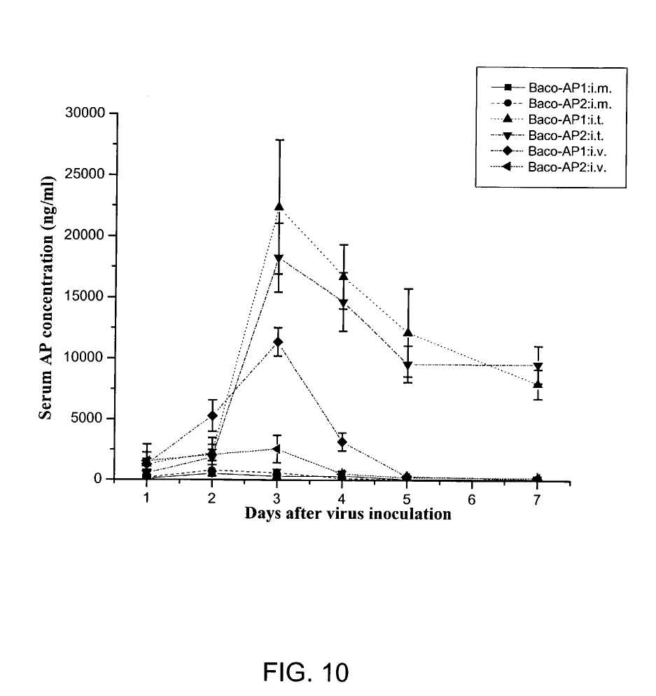

Tumor selective gene expression of UL38p in the context of oncolytic HSV in vivo was demonstrated. A human xenografted liver tumor (Hep 3B) was established subcutaneously on the right flank of athymic nude mice. Once the tumor size reached around 8 mm in diameter, the viruses (Baco-AP1 or Baco-AP2) were injected intratumorally at a dose of 5×10⁶ pfu. At the same time, mice that did not receive tumor inoculation were injected with the same amount of the viruses either by intravenous (i.v.) or intramuscular (i.m.) injection. Blood was collected at different time points after virus injection and quantified for the AP release. The AP concentration in the mice injected intratumorally with either of the viruses started to increase by day 2 after virus administration, and reached their peak level by day 3. The AP release started to decline afterwards, but was maintained at a relatively high level for the rest of the experiment (FIG. 10). The AP release from Baco-AP1 was marginally higher than that of Baco-AP2 during the entire experiment except on the last time point (day 7) when the former was slightly lower than the later (P>0.05). By contrast, there were significant differences on the AP release at day 2, 3 and 4 after the viruses were injected intravenously (P<0.01). Intravenous injection of Baco-AP2 only produced a slight increase of AP in the blood of the animals at day 2 and day 3. By day 4 after virus administration, the AP returned to the background level and then stayed at the low level for the rest of the experiment. However, i.v. injection of the same amount of Baco-API produced a much higher AP release in the serum before day 5 after virus inoculation. AP in the blood of mice receiving either of the viruses intramuscularly stayed at the basal level for the entire experimental period, which is probably due to poor transduction of myoblasts in adult mice by HSV vectors. Together, these results demonstrate that in the context of an oncolytic HSV, UL38p can direct a strong and tumor selective gene expression during its in vivo administration.

### Example 10

### Exemplary Materials and Methods for Examples 11-13

Cell Lines Mice

African green kidney (Vero) cells were purchased from American Tissue Culture Collection (Rockville, Md.). The human cell line HEY and SKOV3 were kindly gifted from Dr. Robert Bast (MD Anderson Cancer Center, Houston, Tex.). Hey cell was established from a peritoneal deposit of a moderately differentiated papillary ovarian cystoadenocarcinoma, and it has been reported to be moderately resistant to chemotherapeutic agent (Selby et al., 1980; Buick et al., 1985). All cells were cultured with DMEM containing 10% fetal bovine serum (FBS) and antibiotics in a humidified 5% CO₂ atmosphere. Female Hsd athymic (nu/nu) mice (obtained from Harlan, Indianapolis, Ind.) were bred under specific pathogen-free conditions and used for experiments at age of 7 to 8 weeks.

Construction of Fusogenic HSV Vectors

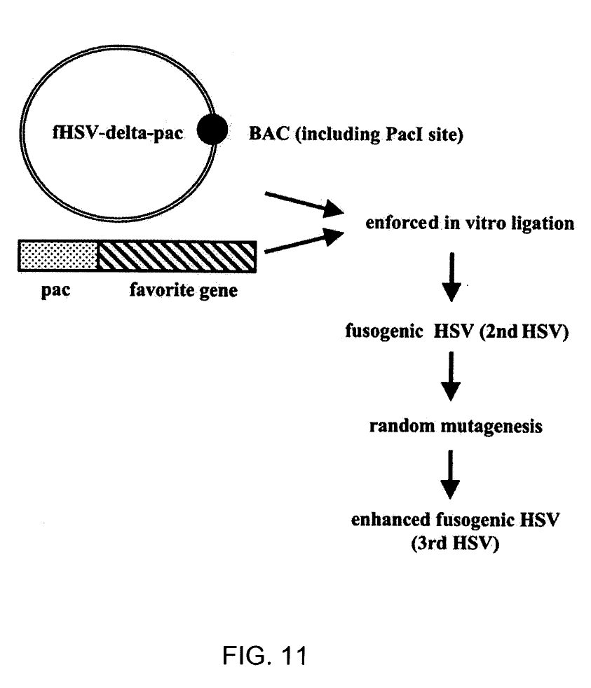

All oncolytic HSVs were derived from fHSV-delta-pac, a bacterial artificial chromosome (BAC)-based construct that contains a mutated HSV genome, in which the diploid gene encoding γ34.5 and both copies of HSV packaging signal (pac) have been deleted (Saeki et al., 1998). Infectious HSV cannot be generated from this construct unless an intact HSV pac is provided in cis. To insert from foreign genes and to generate infectious viruses from fHSV-delta-pac, we used an enforced ligation strategy (FIG. 11). First, two gene cassettes (GALV.fus driven by the UL38 promoter, and the green fluorescent protein gene driven by the CMV promoter) were cloned into pSZ-pac so that each was linked with a HSV sequence containing HSV pac. The EGFP gene cassette was cut from pEGFP-N1 (Clontech, Palo Alto, Calif.) with AseI and AflII. The GALV.fus gene was cut from pCR3.1-GALV and linked with the promoter and enhancer region (DAS) of UL38, which has been well defined (McGuire et al., 1996). The GALV.fus or EGFP gene cassette that was linked with the HSV pac, was then cut from the plasmids and directly ligated into the unique PacI site of fHSV-delta-pac. The ligation mixture was directly transfected into Vero cells using LipofectAMINE (Gibco-BRL) and incubated for 3-5 days for infectious virus to be generated. The viruses were subsequently plaque purified were designated Baco-1 (containing the DNA fragment of EGFP and pac) and Synco-2 (containing UL38-GALV.fus and pac). And then, to generate the enhanced fusogenic potent HSVs, random mutagenesis of Synco-2 was performed according to a procedure published previously (Fu and Zhang, 2002; Schaffer et al., 1973). Viral stocks were prepared by infecting Vero cells with the virus at 0.01 plaque-forming units (pfu) per cell, harvested after 2 days and stored at −80° C.

Membrane fusion capability was first introduced into a conventional oncolytic HSV, Baco-1 (2), through random mutagenesis of incorporation of the thymidine analogue BrdU during viral replication in Vero cells (Fu and Zhang, 2002). The virus was phenotypically identified and purified to homogeneity. The gene encoding the hyperfusogenic GALV.fus, driven by the strict late promoter of the UL38 gene, was then cloned into the BAC-based viral genome through an enforced ligation strategy, to replace the enhanced green fluorescent protein gene of Baco-1. One of the plaque-purified viruses, designated Synco-2D, was chosen for further characterization.

Characterization of Synco-2D and In Vitro Cell Killing Assay

Hey or SKOV3 ovarian cancer cells were seeded into 6-well plates, and then infected the following day with Baco-1 or Synco-2D at a dose of 0.01 pfu/cell. Cells were cultured in a maintenance medium (containing 1% FBS) and were left for up to 2 days to allow the fusion pattern and plaques to develop. To measure in vitro killing effect of viruses, Hey or SKOV3 tumor cell lines were seeded into 24-well plates and were infected with Baco-1 or Synco-2D at 0.1 and 0.01 pfu/cell, or were left uninfected. Cells were harvested 24 h or 48 h later through trypsinization. The number of viable cells was counted on a hemocytometer after trypan blue staining. The percentage of cell viability was calculated by dividing the number of viable cells from the infected well by the number of cells from the well that was left uninfected. The experiments were done in triplicate, and the averaged numbers were used for the final calculation.

Experimental Design of In Vivo Virotherapy for Orthotopic Ovarian Cancer Models

To produce tumors, Hey cells were harvested from subconfluent cultures by a brief exposure to 0.25% trypsin and 0.05% EDTA. Trypsinazation was stopped with medium containing 10%FBS, and cells were washed once in serum-free medium and resuspended in PBS. Only single cell suspensions with >95% viability were used for the in vivo injection. Briefly, on day 0, 3×10⁵ viable Hey cells were inoculated into the peritoneal cavities of 8 weeks old female nude mice. On day 14 and 28 after tumor inoculation, intraperitoneal administration of HSV vector (2×10⁷ pfu/200 μl) was initiated at a different site from tumor cells inoculated. To provide objective, blinded data, mice were implanted with Hey tumor and randomized into three groups of eight animals. These groups included; (a) controls to which phosphate-buffer saline (PBS) was administered; (b) mice treated on day 14 and 28 with Baco-1; and (c) mice treated on day 14 and 28 with Synco-2D. On day 42 after tumor cell inoculation, mice were euthanized by CO₂ exposure and subjected to macroscopic (number of tumor nodule, tumor volume) analysis in a blinded fashion.

Statistical Analysis

Quantitative results are expressed as mean standard deviation of the mean. The statistical analysis was performed by Student's t test or one-way ANOVA, with the exception of the survival data that were analyzed by the Kaplan-Meier plot and the log-rank (Mantel-Cox) test using Statview 5.0 software (Abacus Concepts, Berkeley, Calif.). P values less than 0.01 were considered significant in this study.

### Example 11

### Generation and Characterization of Newly Fusogenic HSV Vector

In specific embodiments of the present invention, the inventors sought to overcome deficiencies in the art including reduced potency of viruses in tumors due to deletions that confer viral replication selectivity and, unlike other therapeutic strategies (such as prodrug enzyme delivery), the antitumor activity of oncolytic HSV does not potentiate a significant bystander effect. The bystander effect is considered to be crucial for effective antitumor therapy for advanced cancers, particularly ovarian, because it compensates for the limited efficiency of vector delivery and spread in, for example, the abdominal cavity. Therefore, it is likely that additional improvements on both the potency and killing ability of these oncolytic viruses is a requisite to obtaining a clear clinical benefit for cancer treatment, particularly for ovarian cancer.

To overcome these problems, employing the natural cellular membrane fusion activity of viral fusogenic membrane glycoproteins (FMGs) is an additional possible method to accomplish virus dispersion. Historically, FMGs were expressed from their native viruses such as influenza virus, measles virus, and vesicular stomatitis virus. These fusogenic viruses were used therapeutically to induce oncolysis (Bateman et al., 2000). More recently, recombinant DNA forms of FMGs have been expressed from plasmid and heterogeneous viral vectors (Hhiguchi et al., 2000; Diaz et al., 2000). Importantly, expression of the recombinant protein from these vectors has been shown retain the biological activity of the FMGs as antitumor agents. Furthermore, the bystander killing effect from this protein is at least 10 times higher than the effect from the suicide gene/prodrug system such as HSV-TK/GCV or CD/5-FC (Higuchi et al., 2000).

Hence, the present inventors have modified the fusogenic potency of oncolytic HSV. In specific embodiments, the present inventors have generated a new type of fusogenic HSV vector by combination of random mutagenesis and insertion of GALV.fus. To determine whether the virus killed widely for ovarian tumor cells, the extent of syncytial formation induced by HSV infection was examined in vitro. After that, for the purpose of a providing a novel agent for intraperitoneal therapy of ovarian cancer, the antitumor efficacy of the virus was evaluated in an orthotopic model of advanced ovarian cancer in athymic mice.

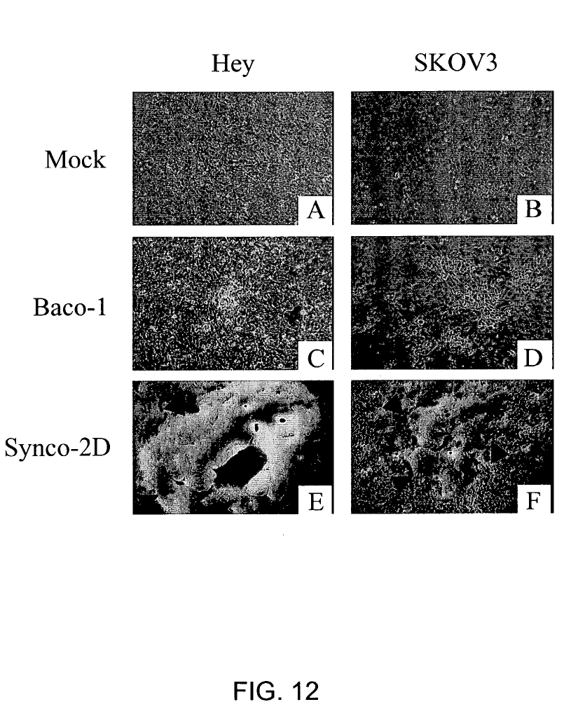

Synco-2 was subjected to random mutagenesis through incorporation of the thymidine analogue BrdUrd during its replication in Vero cells. The mutagenized virus stock was then screened for the ability to confer a syncytial phenotype on infection of Vero cells. Plaques that were predominantly formed from syncytial formation were collected, and one isolate, designated Synco-2D, consistently showed a strong syncytial phenotype after a few passages. After then, this virus purified to homogeneity through multiple rounds of plaque purification from 100% of plaques displayed a syncytial phenotype. To characterize phenotypically the newly isolated oncolytic virus, Hey and SKOV3 ovarian tumor cells were infected with Baco-1 or Synco-2D at 0.01 pfu/cell. As shown in FIGS. 12E and 12F, the syncytial phenotype of plaques after Synco-2D infection was strikingly different from the ordinary plaques derived from infection of Baco-1. Actually, by 24 h after infection, the infection foci of Baco-1 were relatively small and were composed mainly of round cells that are characteristic of a standard HSV infection. In contrast, the plaques from Synco-2D infection were composed entirely of cells that were fused together, such that the boundaries of individual cells were almost invisible. Each plaque from Synco-2D infection covered an area equivalent to several hundred cells, in sharp contrast to those from Baco-1 infection in which substantially fewer cells were involved. By 48 h, the infection foci from Baco-1 infection had gotten larger, but by 72 h the monolayer still had not reached 100% CPE. However, the cells infected by Synco-2D displayed a markedly different phenotype; by 48 h, the cells in the entire dish were fused together (and appeared like a “fishing net”), and by 72 h, the cells appeared contracted (resembling a “a big lake”). These phenomenon showed that Synco-2D was phenotypically different from the parental Baco-1 virus and, because of its additional cell membrane fusion feature, had a much stronger cell-killing ability than Baco-1.

### Example 12

### Comparison of In Vitro Tumor Cell Killing

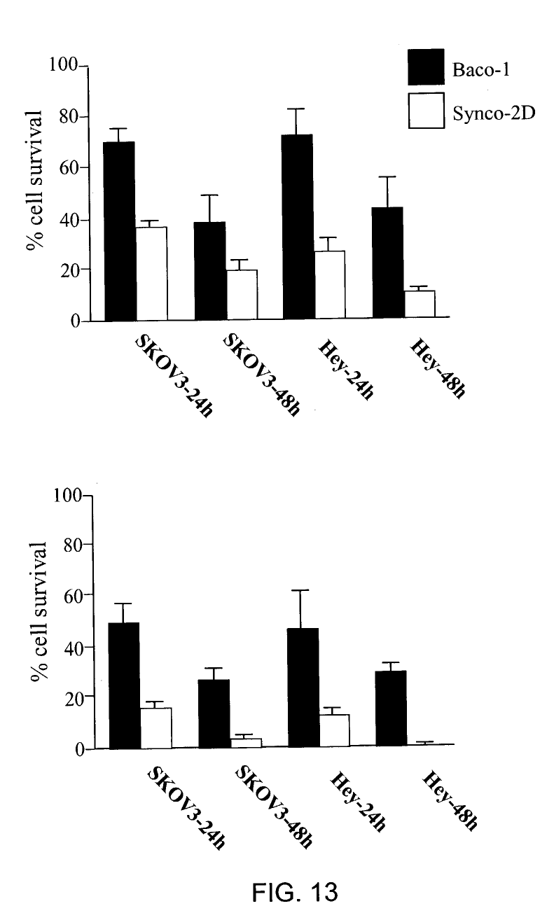

The ability of Synco-2D to destroy tumor cells was further characterized. The cells were infected with Baco-1 or Synco-2D at a relatively low multiplicity of infection (0.1 and 0.01 pfu/cell), which assesses both the inherent cytotoxicity of the input virus as well as the ability of the virus to replicate and spread in these cells. The cytotoxic effect of the virus infection on the tumor cells was quantified by calculating the percentage of cells that survived after the virus infection. In Hey and SKOV3 cell lines, Synco-2D had a significantly stronger ability to kill these tumor cells than Baco-1 (p<0.01). At an infection dose of 0.01 pfu/cell, Synco-2D suppressed the cell viability under 50% within 24 h (FIG. 13A), moreover, this was particularly evident at an infection dose of 0.1 pfu/cell (FIG. 13B).

### Example 13

### Therapeutic Efficacy of Newly Modified Fusogenic HSV Vector for Orthotopic Ovarian Cancer Model

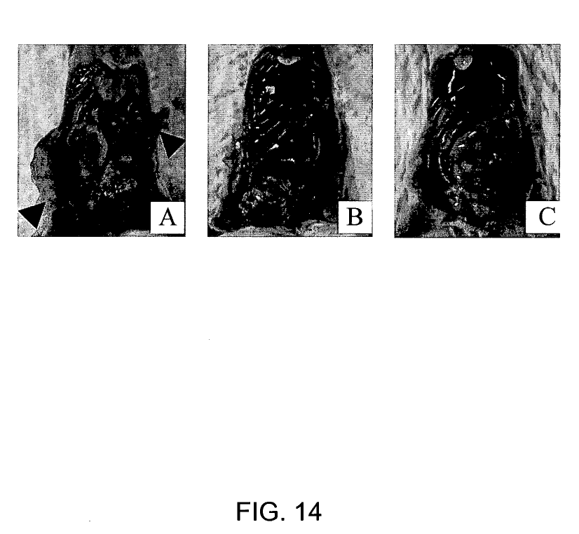

In an orthotopic model of ovarian cancer, 3×10⁵ viable HEY cells inoculated into the peritoneal cavities of nude mice. In this model, no macroscopic or histopathological evidence of a tumor was observed in control mice that were sacrificed either 2 or 7 days after intraperitoneal inoculation of Hey cells, described previously (Auzenne et al., 2002). However, after 14 days of tumor inoculation when the HSV vectors were administered intraperitoneally, tumors had become well established. Actually, for implanted with Hey tumors, the first sign of tumor growth was needle track in the muscle of the abdominal wall, and eventually palpable intraperitoneal tumor through abdominal wall was evident (the tumor diameters approximated 3 mm) after 14 days of tumor inoculation. As the latter progressed, cachexia became more significant, especially in PBS treated mice. These morbid symptoms eventually required humane sacrifice. They did not display prominent abdominal distention from ascites. On occasion, mice bearing either tumor succumbed between daily observation and before the opportunity to sacrifice them. In this case, the day of death was considered to be the day before the date they were discovered. All the PBS-treated mice died within 40 days (36.5±0.7 days) because of cachexia or intraperitoneal dissemination (FIG. 14A). In addition, only PBS-treated mice appeared intraperitoneal dissemination (at number of 2.5±0.9) and the peritoneal thickness (Table 5, FIG. 14A).

TABLE 5
Fusogenic oncolytic HSV therapy for ovarian cancer in the peritoneum

| | | | | tumor | | tumor |
|---|---|---|---|---|---|---|
| Treatment | N | incidence | nodule | weight (g) | death | free |
| PBS (control) | 8 | 8/8 | 2.5 ± 0.9 | 1.0 ± 0.6 | 8/8 | 0/8 |
| Baco-1 | 8 | 8/8 | 1.0 ± 0.0^{b} | 1.5 ± 0.7 | 3/8^{b} | 0/8 |
| Synco-2D | 8 | 2/8^{a} | 0.25 ± 0.4^{a} | 0.1 ± 0.3^{a} | 0/8^{a} | 6/8^{a} |

Hey ovarian cancer cell (3×10⁵) were inoculated into the peritoneal cavity of nude mice.

After 14 days of Hey cell inoculation, group of mice were treated with once per two weeks

intraperitoneal administration of PBS or Baco-1 or Synco-2D.

^{a}p<0.01 as compared with the other group; ^{b}p<0.01 as compared with control

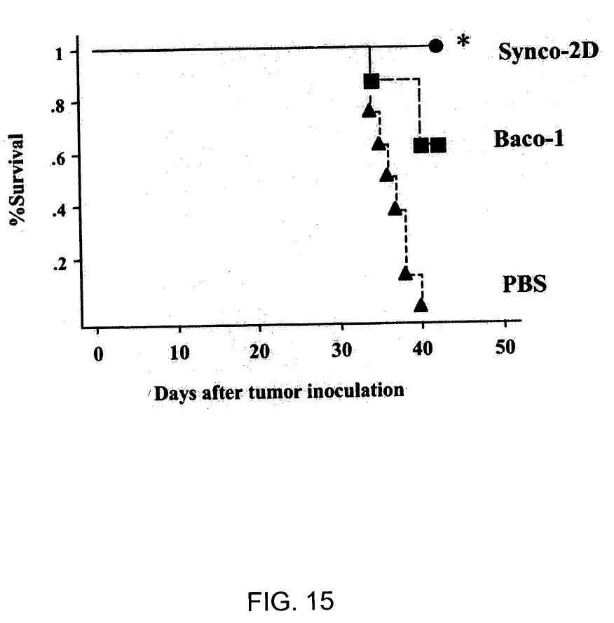

On the other hand, three of Baco-1 treated mice died till the euthanized day, and the residual five mice bore a single but large tumor as same as the PBS-treated mice at the euthanized day. Surprisingly, only two mice developed much smaller tumors. There was statistically significant difference in the number or weight of tumors from other two groups (p<0.01) (Table 5). Moreover, the beneficial therapeutic effects of the administration with Synco-2D were also reflected on the complete remission from cancer state and survival prolongation (Table 5, FIG. 15). There was also statistically significant difference from the other group (p<0.01).

### Example 14

### Exemplary Materials and Methods for Examples 15-17

Cell Lines and Viruses

African green monkey kidney (Vero) cells were purchased from American Tissue Culture Collection (Rockville, Md.). Vero cells were cultured with DMEM containing 10% fetal bovine serum (FBS) and antibiotics in a humidified 5% CO₂ atmosphere. PC-3M-Pro4 cell is a highly metastatic prostate cancer cell line (Pettaway et al., 1996). This cell line was grown in RPMI 1640 supplemented with 10% FBS. For in vivo inoculation, cells were harvested from culture flasks after a brief trypsinization. Only single-cell suspensions of >95% viability (trypan blue exclusion) were used for inoculation.

The oncolytic HSVs were derived from fHSV-delta-pac, a bacterial artificial chromosome (BAC)-based HSV construct, and the details for their construction has been described elsewhere (Fu et al, in press, Molecular Therapy; Nakamori et al, in press, Clinical Cancer Research). Briefly, to generate Baco-1 and Synco-2, the enhanced green fluorescent protein gene (EGFP, for Baco-1) or GALV.fus (for Synco-2), together with the HSV ligation mixture was subsequently transfected into Vero cells for virus production. To generate Synco-2D, Baco-1 was initially subjected to random mutagenesis followed by screening for the syncytial phenotype as described (Fu and Zhang, 2002). After that, the circular form of viral DNA was obtained by extracting virion DNA from Vero cells shortly (1 h) after virus infection, through a method previously described (Zhang et al., 1994). The viral DNA was then transformed into competent E. coli cell DH-10B through electroporation. The gene cassette encoding green fluorescent protein (GFP) in the Baco-1 viral genome was then cut out with PacI and replaced with GALV. fus (driven by the conditional UL38 promoter of HSV) through an enforced ligation strategy as described (Fu et al., in press, Molecular Therapy). The ligation mixture was directly transformed into Vero cells using LipofectAMINE (GIBCO-BRL) and incubated for 3-5 days for infectious virus to be generated. The virus was subsequently plaque purified. Viral stocks were prepared by infecting Vero cells with the viruses at 0.01 plaque-forming units (pfu) per cell, harvested after 2 days and stored at −80° C. The viral titers were quantified by plaque assay and were represented as plaque forming units (pfu).

In Vitro Phenotypic Characterization and Cell Killing Assay

For in vitro phenotypic characterization of the fusogenic oncolytic HSVs, PC-3M-Pro4 cancer cells were seeded into 6-well plates. The cells were infected the following day with serially diluted viruses (Baco-1, Synco-2, or Synco-2D) and were cultured in a maintenance DMEM medium (containing 1% FBS) for up to 3 days to allow the fusion pattern and plaques to develop. To evaluate the cytotoxicity of each virus in vitro, PC-3M-Pro4 cells were plated at 5×10⁴ cells/well in 12-well plate. Cells were infected by Baco-1, Synco-2 or Synco-2D at 0.01 and 0.1 pfu/cell, respectively. Cells were harvested at 24 h intervals, and the cell viability was determined using trypan blue staining. The percentage of cell viability was calculated by dividing the number of viable cells from the infected well by the number of cells from the well that was left uninfected. The experiments were done in triplicate, and the averaged numbers were used for the final calculation.

Animal Studies

BALB/cByJIcrSmnHsd-scid mice (7-8 week old) were obtained from Harlan (Indianapolis, Ind.) and were kept in groups of four or fewer under specific pathogen-free conditions. All animal studies were approved Baylor College of Medicine Animal Care and Use Subcommittee and were performed in accordance with its policies. For surgical procedures, all of the mice were anesthetized with an intraperitoneal injection of mixture solution containing 2.5% 2,2,2-tribromoethanol and tert-amylalcohol (1:1). Orthotopic inoculation was performed according to a procedure described in the literature (Stephenson et al., 1992). Briefly, a transverse incision was made in the lower abdomen. After the abdominal wall muscles were split, the bladder and seminal vesicles were exposed and retracted anteriorly to reveal the dorsal prostate. Then, 2×10⁵ PC-3M-Pro4 cells suspended in 10 μl of PBS were carefully injected under prostate capsule using a 30-gauge needle and glass Hamilton syringe (Hamilton Syringe Co., Reno, Nev.). Formation of a bulla indicated a satisfactory injection. The incision was closed with a single layer of surgical clips (Autoclip; Clay Adama, Parsippany, N.J.). Lung metastases of prostate cancer were established through tail vein injection of PC-3M-Pro4 (1×10⁵/100 μl) at the next day after orthotopic tumor inoculation (Hull et al., 2000). Mice were then randomly divided into 4 groups (n=8) and were injected through tail vein twice with either PBS (as control), or with 2×10⁷ pfu of viruses (Baco-1, Synco-2, or Synco-2D, at a volume of 100 μl), on 7 and 14 days after the tumor inoculation. Forty days after orthotopic tumor inoculation, mice were euthanized by CO₂ inhalation. Primary tumors were excised and weighted. At the same time, animal lungs were resected, washed in saline, and placed in Bouin's fixative. Lung metastases were counted with the aid of a dissecting microscope 24 h later as described previously (Shevrin et al., 1989).

Statistical Analysis

Quantitative results are expressed as mean±standard deviation. The statistical analysis was performed by one-way ANOVA using Statview 5.0 software (Abacus Concepts, Berkeley, Calif.). P values less than 0.05 were considered significant in this study.

### Example 15

### Syncytial Formation of Fusogenic Oncolytic HSVS in PC-3M-PRO4 Prostate Cancer Cells

In the following Examples, the antitumor efficacy of different versions of oncolytic HSVs was evaluated in a murine model bearing both primary tumor and lung metastasis of human prostate cancer xenografts, which were established through simultaneous orthotopic and systemic injection of the human prostate cancer cell line, PC-3M-Pro4. The data demonstrated herein that oncolytic HSVs, such as, for example, Synco-2D, is a potent therapeutic agent on this exemplary tumor model and that intravenous administration of this virus led to a significant shrinkage of the primary tumor and a dramatic reduction of tumor nodules in the lung. These results indicate that systemic administration of this potent fusogenic oncolytic HSV is an effective treatment for metastatic human prostate cancer.

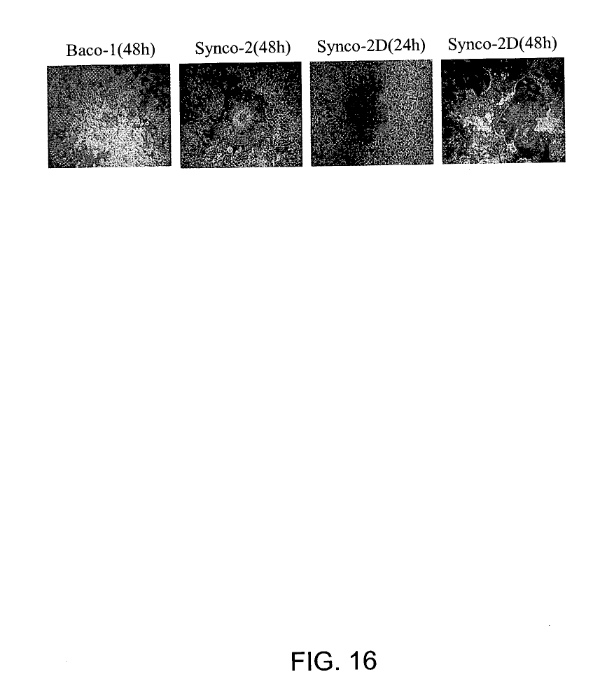

The exemplary human prostate cancer cell line PC-3M-Pro4 was chosen for both in vitro and in vivo experiments. This cell line was selected from PC-3M through repeated cycles of orthotopic inoculation/harvest in athymic mice, and has been shown to efficiently establish lung metastases after intravenous injection into immune deficient mice (Pettaway et al., 1996). To phenotypically characterize Synco-2D on human prostate cancer line PC-3M-Pro4, the cells were infected with serially diluted viruses (Baco-1, Synco-2, or Synco-2D). At different times after infection, photos were taken from a typical plaque formed by virus infection. As shown in FIG. 16, while infection of Baco-1 produced typical plaques that were formed from round cells, plaques from Synco-2 or Synco-2D infection were phenotypically different and were composed entirely of cells that were fused together. The plaque from Synco-2D infection was significantly larger than the one from Synco-2 or Baco-1. The plaque size from 24 h infection is almost equivalent to that of 48 h infection from Synco-2. By 48 h after infection, the plaque size was too big to be taken in a single microscopic field. This result indicates that, due to its double fusion capability, Synco-2D may have a stronger tumor-cell killing ability than the singly fusogenic oncolytic HSV Synco-2.

### Example 16

### Direct Comparison of Singly and Doubly Fusogenic Oncolytic HSVS on Killing Prostate Cancer Cells

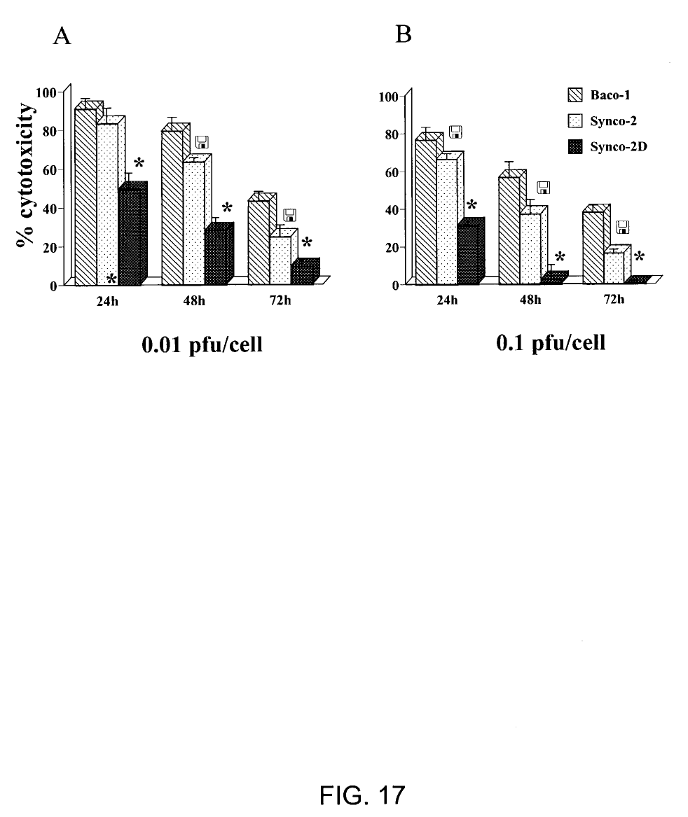

To demonstrate that the enhanced ability to induce syncytial formation of Synco-2D correlates with an increased ability to destroy tumor cells, PC-3M-Pro4 cells were infected with Baco-1, Synco-2, or Synco-2D at a relatively low multiplicity of infection (0.1 and 0.01 pfu/cell), which assesses both the inherent cytotoxicity of the input virus as well as the ability of the virus to replicate and spread in these cells. The cytotoxic effect of the virus infection on the tumor cells was quantified by calculating the percentage of cells that survived after the virus infection against the cells in a well that received no virus infection. The results showed that as compared with Baco-1, both fusogenic oncolytic HSVs have a significantly higher cytotoxic activity in this tumor cell line at both doses and the time points of harvest, with the only exception of the early time point (24 h) and the lower viral dose (0.01 pfu/cell) (FIG. 17). Direct comparison between Synco-2 and Synco-2D revealed that the doubly fusogenic HSV had a significantly stronger cytotoxic ability than the singly fusogenic virus at all the time points of either viral doses (P<0.01). At an MOI as low as 0.01 pfu/cell, Synco-2D infection reduced the cell viability to less than 50% within 24 h. The virus-mediated cytotoxicity was more evident at the higher dose of virus infection (0.1 pfu/cell). Synco-2D infection resulted in complete destruction of the tumor cells at 72 h after infection when the cells were initially hit with 0.1 pfu/cell. These results indicate that incorporation of an extra cell-membrane fusion mechanism into a singly fusogenic oncolytic HSV can further enhance the ability of the virus to destroy tumor cells in vitro.

### Example 17

### Therapeutic Efficacy on Primary Tumor After Systemic Delivery

To evaluate the potency of these fusogenic oncolytic HSVs on human prostate cancer, both primary and metastatic xenografts were established through orthotopic and systemic injection of PC-3M-Pro4 into SCID mice. Eight days after tumor cell implantation, five mice were checked surgically and all of them were found to have primary tumor formation at sizes around 2 mm in diameter. At that time point, mice were given first intravenous injection (through tail vein) of either oncolytic viruses (Baco-1, Synco-2 or Synco-2D) at a dose of 2×10⁷ or the same volume (00 μl) of PBS as a control. A repeated injection with the same dose of virus was given one week later. Forty days after the initial inoculation of PC-3M-Pro4 cells, orthotopic tumors were explanted and weighted. Regional lymph node metastasis and lung metastasis were identified and numerated macroscopically with a dissecting microscope. Three mice from PBS-treated group died before the end point of the experiment (at day 33, 35 and 36 after initial tumor inoculation, respectively). The tumor volume and the metastatic nodules in these 3 mice were determined the same way as described and the data were combined with the data obtained from the rest of mice that were sacrificed by the end of the experiment.

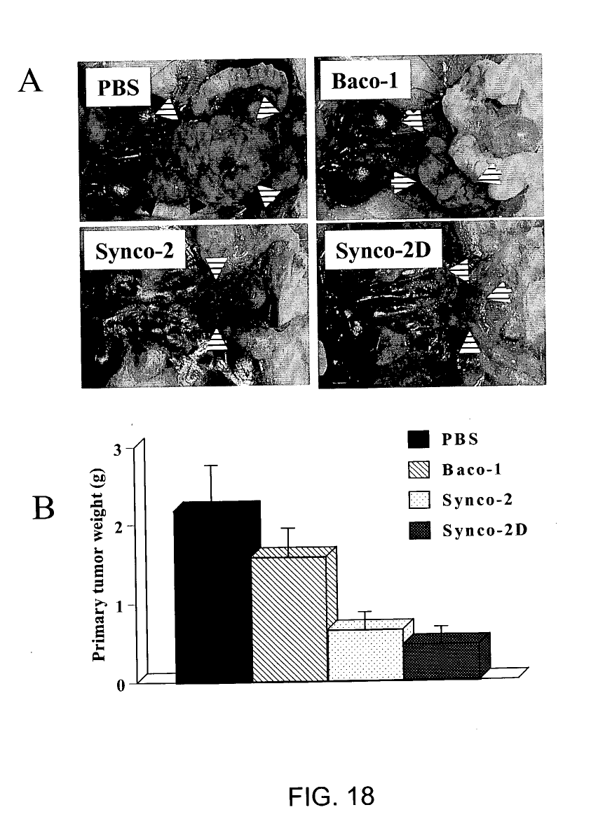

The results showed that systemic delivery of oncolytic HSVs had a significant therapeutic effect on the growth of the established prostate cancer on the primary site. By the time the animals were sacrificed (or the time when the animals died due to tumor overgrowth), the orthotopic tumor in PBS-treated mice reached a very large volume, with an average weight of 2.17±0.59 mg (FIG. 18). Systemic administration of a conventional oncolytic HSV Baco-1 produced noticeable suppression on the growth of the orthotopic tumor. The average weight of the orthotopic tumor in this group of mice is 1.57±0.36 mg, which represents almost 30% reduction of the tumor size over the PBS control group. The therapeutic effect on the orthotopic tumor from the fusogenic oncolytic HSVs is even more profound: the average tumor weight in mice treated with either Synco-2 or Synco-2D is less than half of those in Baco-treated mice. Although the tumor volume from Synco-2D-treated mice is even smaller than that in mice treated with Synco-2 (0.45±0.21 mg Vs. 0.64+0.22 mg), this difference is not statistically significant (p=0.287). These results revealed that both Synco-2 and Synco-2D are significantly more effective than Baco-1 on reducing orthotopic tumor burden after systemic administration, and that Synco-2D is only marginally better than Synco-2 against the established orthotopic tumor.

Systemic administration of oncolytic HSVs also produced dramatic therapeutic effects on the lung metastases of prostate cancer (Table 6).

TABLE 6
Therapeutic effect of oncolytic
HSVs on lung metastases of PC-3M-Pro4

| Treatment | PBS (control) | Baco-1 | Synco-2 | Synco-2D |
|---|---|---|---|---|
| Lung | 25.4 ± 12.2 | 12.5 ± 3.1^{a} | 6.8 ± 2.2^{a,b} | 1.1 ± 1.6^{a,b,c} |
| metastases | | | | |
| LN | 1.6 ± 0.9 | ND | ND | ND |
| metastases | | | | |

Lung metastases were established through tail vein injection of PC-3M-Pro4 (1×10⁵/100 μl), which was given one day after orthotopic tumor inoculation. The therapeutic scheme was the same as described in FIG. 18. Forty days after orthotopic inoculation, mice were euthanized by CO₂ inhalation and their lungs were resected, washed in saline, and placed in Bouin's fixative. After then, lung metastases were counted with the aid of a dissecting microscope 24 h later.

ND: not detected; ^{a}p<0.01 μl as compared with control; ^{b}p<0.01 as compared with Baco-1; ^{c}p<0.05 as compared with Synco-2

Compared with mice treated with PBS, where an average of 25.4±12.2 tumor nodules per lung were recorded, mice that were given Baco-1 only had half of the metastatic nodules in their lung. The most dramatic result was obtained in the group where mice received Synco-2D administration. On average, only 1.111.6 tumor nodules per lung were found in the animals in this group, which is significantly less than the number of lung metastatic nodules (6.8±2.2) recorded in animals treated with Synco-2 (p=0.0424). On the other hand, both Synco-2 and Synco-2D had a significantly better therapeutic effect on lung metastasis than Baco-1 (p<0.01). Tumor metastasis to the local draining lymph nodes was also assessed at the endpoint of the treatment. The result showed that administration of either Baco-1 or any of the fusogenic oncolytic HSVs effectively prevented the lymph node invasion of the orthotopic tumor, as the tumor metastasis to the local lymph nodes was only detected in the PBS control group. Together, these results demonstrate that in correlation with its increased ability to kill cultured tumor cells, the doubly fusogenic oncolytic HSV is more potent than the singly fusogenic oncolytic HSV at treating lung metastasis of prostate cancer after systemic administration.

### Example 18

### Tumor Destruction by Fusogenic Oncolytic HSV Induces Strong Antitumor Immunity

Immunotherapy is potentially a very useful treatment modality for cancer, as it has the ability to eradicate residual tumors that are difficult to manage by conventional or other gene therapy approaches. However, despite several tumor-associated antigens that have been identified recently (Boon and van der Bruggen, 1996), clinical success using these antigens as vaccines has yet to be shown. Consequently, cancer vaccines derived from whole tumor cells, which theoretically covers the entire repertoire of tumor antigens in a single vaccine preparation, have been extensively exploited (Dranoff et al., 1993; Schadendorf et al., 2000). The success of this whole-cell vaccine approach, either through ex vivo or in vivo manipulation, will largely depend on the development of simple and efficient strategies to stimulate tumor antigen presentation in vivo.

Recently it has been reported that the biochemical mechanisms by which cancer cells are destroyed are of considerable importance to the subsequent immunogenicity of the tumor (Melcher et al., 1998). It is considered that for efficient presentation, the tumor antigen has to be released in such a way that the immune system reacts to it in the context of physiological danger signals—such as uncontrolled tissue destruction or cell death by cell killing mechanisms that can induce necrotic cell death (Matzinger, 2002; Melcher et al., 1999). In particular, it has been shown that introduction of viral FMGs into tumor cells can lead to tumor cell killing through nonapoptotic, autophagic-like mechanisms, which can potentiate antitumor immune response through induction of stress-related proteins (Bateman et al., 2000; Melcher et al., 1998; Basu et al., 2000). Local tumor destruction by syncytia formation has also been shown to be accompanied by release of vesicles reminiscent of exosomes (syncytiosomes), which can load dendritic cells (DCs) more effectively than exosomes from cells dying by other mechanisms, and therefore promotes cross presentation of tumor antigens (Bateman et al., 2002).

In specific embodiments of the present invention, efficient tumor destruction by a fusogenic oncolytic HSV releases a large quantity of tumor antigens, and the unique mechanism of tumor destruction by syncytia formation facilitates the tumor antigen presentation, leading to the generation of a potent antitumor immunity. The combined action of a fusogenic oncolytic HSV (whose direct oncolysis can debulk established breast cancer, for example) and the induced antitumor immunity that can clear the residual tumor, will lead to a complete eradication of established cancer, such as, for example, breast cancer.

Destruction of non-immunogenic murine mammary tumor by a doubly fusogenic oncolytic HSV, but not the non-fusogenic counterpart, indeed induced a high level of cell-mediated antitumor immunity.

Most of murine tumor cells are non-permissive to HSV infection. Among the few murine tumor cell lines moderately permissive to oncolytic HSV infection are N18 (neuroblastoma), MC26 (colon carcinoma) and EJ-6-2-Bam-6a Ras-transformed fibroblasts (Chahlavi et al., 1999; Yoon et al., 2000; Lambright et al., 2000). To identify murine mammary tumor cells suitable for establishing tumors in syngeneic immune competent mice, a panel of murine mammary tumor cell lines were collected and screened. Among them, 4T1, a 6-thioguanine-resistant cell line derived from a BALB/c spontaneous mammary carcinoma and kindly provided by Dr. Fred Miller (Michigan Cancer Foundation, Detroit, Mich.) (Aslakson et al., 1992), was found to be moderately sensitive to oncolytic HSV infection. However, phenotypic characterization of fusogenic oncolytic HSVs in this cell line showed that only infection of the doubly fusogenic Synco-2D could induce syncytia formation.

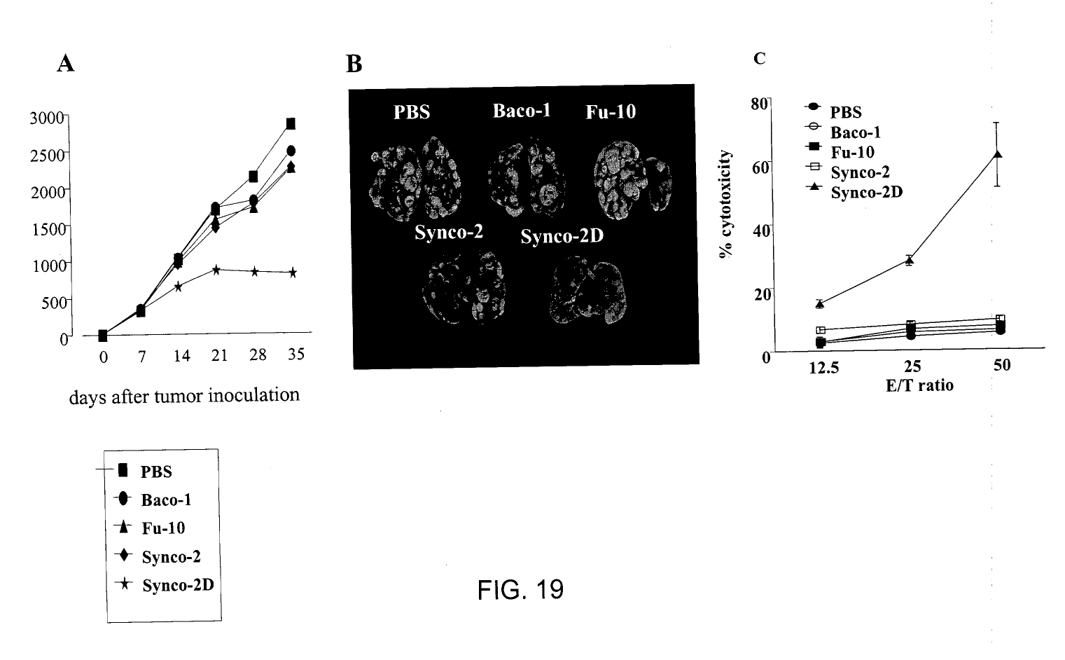

The present inventors also showed that enhanced antitumor effect of Synco-2D on 4T1 tumor is accompanied by elevated tumor-specific CTL activities. 4T1 is non-immunogenic and highly malignant and metastatic in syngeneic BALB/c mice (Aslakson et al., 1992; Pulaski and Ostrand-Rosenberg, 1998). Cells (1×10⁵ 4T1 cells) were orthotopically injected into mammary fat pat of immune competent BALB/c mice for the establishment of primary tumor, and the mice were left for 2 weeks for lung metastases to develop. Tumor-bearing mice were then divided into 3 groups (n=10) and were injected intratumorally with 2×10⁷ pfu of either Synco-2D, Synco-2 or Baco-1, or PBS as a control. Tumor masses on the orthotopic site was measured weekly for 4 weeks. Three mice from each group were sacrificed two weeks after virus injection and spleens were collected for measuring tumor-specific CTL activity (with 4T1 as target cells) by the ⁵¹Cr release assay. The rest of the mice were sacrificed at the end of week 4 and the lung metastatic tumor nodules were enumerated under a dissecting microscope. The results showed that intratumor administration of Synco-2D produced a significantly better therapeutic effect than the singly or non-fusogenic oncolytic HSVs not just on the primary tumor (FIG. 19A), but also on the distant lung metastases (FIG. 19B). By average, there are less than 5 tumor nodules in the lungs from animals treated with Synco-2D, which is in contrast to 42, 38, 34 and 35 tumor nodules seen in mice treated with PBS, Baco-1, Fu-10 and Synco-2, respectively. The enhanced antitumor effect in Synco-2D-treated mice is accompanied by the presence of a high level of tumor-specific cytotoxic T-lymphocyte (CTL) activity. This is in contrast to the mice treated with other viruses in which very little tumor-specific CTL activity was detected (FIG. 19). Although non-fusogenic oncolytic HSV G207 has been shown in earlier studies to induce tumor-specific immunity after intratumor inoculation (Walker et al., 1999; Todo et al., 1999), most of the tumor cell lines used in these studies are immunogenic. For the non-immunogenic 4T 1, tumor destruction per se does not induce antitumor immunity. Antitumor immunity was induced only with concurrent delivery of both GM-CSF and IL-2 (Brockstedt et al., 2002). Therefore, the results indicate that the unique mechanism of tumor destruction by the doubly fusogenic oncolytic HSV enhanced tumor antigen presentation, leading to a better antitumor immunity, which may have directly contributed to the enhanced antitumor activity, especially on the distant metastatic tumors.

### Example 19

### Significance of the Present Invention

The present Examples demonstrate that incorporation of a potent FMG, such as a GALV, into an oncolytic HSV can dramatically increase its anti-tumor potency. In specific embodiments, this is because, unlike other viruses such as adenovirus, cell membrane fusion is a natural part of HSV infection process. The virus infection cycle is therefore unaffected by the cloning of the GALV.fus gene into its genome. This allows the syncytial formation and virus replication to work synergistically to destroy tumor cells when the virus reaches tumor sites. In addition, other synergies may also come from the combined actions of these two totally different anti-tumor mechanisms. First, unlike in vitro cultured tumor cells that are relatively homogenous, many tumors contain cells of different lineages, in which some may be resistant to HSV infection due to, for example, lack of viral receptors on the cell surface. The combined anti-tumor action from two completely different mechanisms (direct viral oncolysis and cell membrane fusion) significantly reduces the occurrence of virus-resistant tumor cells, because those cells that become resistant to oncolytic virus infection/replication may be indirectly destroyed by syncytial formation. Thus, in some embodiments, GALV.fus is preferred rather than the FMG from human immunodeficiency virus type 1 (HIV-1), which has recently been cloned into an oncolytic adenovirus, also for the purpose of enhancing oncolytic potency of the virus (Li et al., 2001); FMG from HIV-1 can only induce syncytia in the presence of CD4⁺ target cells, while the GAVL.fus receptor Pit-1 is widely distributed on human cell surface and GALV.fus therefore can target a much wider range of tumor cells. Secondly, although oncolytic viruses can rapidly spread in cultured tumor cells, viral spreading within a solid tumor mass is often limited (Heise et al., 1999). The large size of the viral particles may contribute to the inefficiency of viral spreading in vivo. The cell membrane fusion mediated by GALV.fus may facilitate the spreading of the oncolytic HSV in the tumor mass so that a larger area of the tumor is affected.

Although expression of GALV.fus in the context of an oncolytic HSV provides extra anti-tumor effects of that virus, un-controlled expression of the gene would undoubtedly pose safety concern. This is especially the case when the virus is administered systemically, or injected locally but significantly leaked to the blood stream. One potential way to overcome this problem is to put the GALV FMG gene under the control of a tumor or tissue-specific promoter. Although quite a few tissue and/or tumor specific promoters have been defined and characterized, the common problem with these promoters is that either they are not strictly tumor specific or they lack strong activity when compared with viral promoters. Thus, a strictly late viral promoter in the context of an oncolytic HSV is a strong tumor specific promoter.

The program of HSV gene expression during its lytic infection can be readily divided into early and late phases. The early genes are transcribed prior to viral DNA replication while the late genes are expressed at high levels only after viral DNA replication has taken place. There are subdivisions within each phase. A few genes in the late phase, including UL38, are classified as strictly late, which are only reliably detectable after the onset of viral DNA replication. The initial characterization on a secreted form of alkaline phosphatase showed that without productive HSV replication, the UL38 promoter activity is extremely low. However, the UL38 promoter activity is almost equivalent to CMV IE promoter once the virus is undergoing lytic infection. The expression of GALV.fus gene driven by UL38 promoter in Synco-2 is also strictly tailored to the full replication cycle of the virus in the tumor cells. In the quiescent normal embryonic fibroblasts, Synco-2 mediated cell fusion is totally ablated. This indicates that Synco-2 is probably no more harmful to normal cells than the first generation oncolytic HSV, whose safety record has been well established in both pre-clinical studies and phase I clinical trails.

Oncolytic viruses developed from HSV have shown great utility for treating solid tumors and are currently in clinical trial for patients with brain tumors (Markert et al., 2000; Rampling et al., 2000); they are also suitable vectors for delivering therapeutic genes for cancer treatment. Compared to traditional defective viral vectors, conditionally-replicating vectors have several advantages. First, unlike the defective vectors that are merely functioning as a delivering vehicle, oncolytic HSVs themselves possess a very high therapeutic index against the tumor. Any anti-tumor effect from the delivered therapeutic gene should be additive and lead to a higher total therapeutic effect. Secondly, as the oncolytic viruses have the ability to spread in the tumor tissues, they can deliver the therapeutic genes to a larger area of tumor mass than defective viral vectors. Thirdly, the ability of the virus to replicate in the tumor may lead to a longer period of gene expression. In support of this, the Examples showed that after intratumor delivery of oncolytic HSV, the AP expression was maintained at a relatively high level over one week, which is in contrast to gene expression from defective HSV vectors that usually lasts for only 24 to 48 h after intratumor injection (Todryk et al., 1999).

Because most of the therapeutic genes used for tumor therapy are also potentially toxic to normal cells, uncontrolled expression of these genes, even in the context of a tumor-restricted oncolytic virus, still poses a safety concern. This is particularly true when systemic administration is required (e.g., for metastatic diseases). One way of minimizing this concern is to use a tumor- or tissue-specific promoter to control the gene expression. While several tissue-specific promoters have been described for tumor specific gene expression, they generally have much lower activity than viral promoters. The Examples provided herein showed that a strict late viral promoter is a strong and tumor specific promoter in the context of an oncolytic HSV. This was demonstrated by both in vitro and in vivo characterization of the promoter of the strict late gene UL38 of HSV. UL38p has very low basal activity that is maintained in non-dividing cells infected with an oncolytic HSV carrying the UL38p cassette. However, in cycling cells where the oncolytic virus can replicate, UL38p showed a similar level of activity as the CMV-P. The cell-cycle dependent property of UL38p was also demonstrated in vivo. Intratumoral injection of both Baco-AP1 and Baco-AP2 produced high level AP expression. On the other hand, only a transient and low level of AP expression was detected during i.v. injection of Baco-PA2, which is in contrast to the abundant AP release from i.v. administration of Baco-AP1. The transient and low level of AP release after i.v. injection of Baco-AP2 was probably from certain dividing cells such as fibroblasts which might have gained access to the virus and were subsequently infected after systemic delivery of the virus. However, since the majority of the viral particles are distributed to liver after systemic delivery (Schellingerhout et al., 1998; Wood et al., 1999), the low level and transient AP expression implies that UL38p in the context of an oncolytic HSV has minimal activity in the normal hepatocytes. Both viruses were also delivered intramuscularly to target the mostly post-mitotic myoblasts. However, neither virus produced any appreciable AP release after this route of injection. This is probably because not enough myoblasts were transduced by either of the viruses, as it has been reported that the mature basal lamina of muscles from adult mice can prevent HSV from efficiently infecting the myofibers (Huard et al., 1995; Huard et al., 1996).

In addition to having a significantly stronger gene expression, the combination of a strict late viral promoter such as UL38p with an oncolytic HSV has additional advantages over the traditional usage of a tumor-specific promoter in a defective viral vector. Unlike tissue-specific promoters that can only be applied to the tumor of the defined tissue origin, these late viral promoters may be applicable to a variety of tumors in which the virus can conditionally replicate. This is particularly useful for tumors in which tumor specific promoters have not yet been defined. Moreover, conditional activation of the UL38 promoter upon the initiation of lytic HSV infection in the tumor cells may provide a synchronized action between the oncolysis of the virus and the therapeutic effect of the delivered gene. Such a synchronized action may be particularly important under certain circumstance, e.g. in combination with immunotherapy, where the tumor antigen release from the viral oncolysis will be paralleled by the release of immune stimulating molecules from the genes inserted into the virus.

In further embodiments, the combination of random mutagenesis and insertion of GALV.fus gene in a conventional HSV provides more oncolytic efficiency than conventional HSVs for cancer cells, such as, for example, ovarian or prostate cancer cells. In some Examples provided herein, the oncolytic HSV is administered intraperitoneally after 14 days of tumor inoculation, when tumors had become well established in an animal model. Actually, the mice implanted with Hey tumors eventually were palpable through the abdominal wall, and the tumor diameters approximated 3 mm. This tumor-bearing state mimics the advanced ovarian cancer state. After that, two injections of Synco-2D (total dose of 4×10⁷ pfu/400 μl) in mice bearing peritoneal dissemination of Hey cells resulted in complete remission (75%) and survival prolongation compared with Baco-1 and PBS treated mice. A dose of 4×10⁷ pfu of Synco-2D led to almost 100% survival. Taking into account size differences (˜3000 fold), this dose would be the human “equivalent” of 1.2×10¹¹ pfu of virus, a dose similar to the highest dose of G207 administered in Phase I study for malignant gliomas (Markert et al., 2000). These results suggest that treatment of endothelial ovarian cancer at an early stage or in association with a surgical “debulking” procedure to remove gross disease may be safe and successful strategies for Synco-2D gene therapy in humans.

To minimize any potential safety problems, a strict-late viral promoter directs the expression of the GALV.fus gene in the virus, which provides strong tissue-selective expression of the gene. In fact, the UL38 promoter appeared to have several advantages over the conventional tissue-specific promoter, such as HER-2/neu, that is over-expressed in approximately 10% of ovarian cancers and may correlate with a poor prognosis. For example, this promoter is probably substantially stronger than most tissue-specific promoters. In addition, activation of the UL38 promoter upon initiation of lytic HSV infection in the tumor cells provides a synchronized action between these two antitumor mechanisms, in specific embodiments.

Furthermore, a major obstacle to the successful administration of oncolytic viruses is the presence of antiviral immunity. The pre-existing anti-viral immunity can block the initial viral entry as well as its subsequent spread within tumors. Therefore, most cancer patients will have anti-herpes antibodies that might compromise the therapeutic efficacy of the virus. However, neutralizing antibodies would be a more serious concern for systemic therapy than they will be for intraperitoneal therapy of cancer, such as ovarian cancer. Previous studies have shown that the presence of antibodies to replication-competent viruses does not negate the efficacy of therapy or prevent virus replication in the tumor sites (Lambright et al., 2000; Nemunaitis et al., 2000; Chahlavi et al., 1999). Indeed, it has been shown that, despite high levels of circulating antibodies in the serum of mice that received multiple injections of adenovirus, the titer of neutralizing antibodies in the peritoneal cavity was undetectable (Al-Hendy et al., 2000). Also, if neutralizing antibodies are present in ascites, the fluid can be easily aspirated from the peritoneal cavity before therapy. Moreover, it has been recently demonstrated that delivering oncolytic viruses through liposome formation of either BAC-based oncolytic HSV DNA or viral capsids can evade a host's antiviral immune responses (Fu and Zhang, 2000). Thus, intraperitoneal therapy is an attractive route for delivery of the virus to maximize virus/tumor cell interaction and infection.

It was recently demonstrated that syncytial mutant (Fu-10) selected from the well-characterized oncolytic HSV G207 through random mutagenesis has a dramatically enhanced antitumor effect on lung metastasis of breast cancer over the conventional non-fusogenic virus (Fu and Zhang, 2002). Fusogenic oncolytic HSVs constructed from insertion of a hyperfusogenic glycoprotein into a conventional oncolytic HSV can also significantly increase the antitumor effect of that virus. Most recently, a newer version of fusogenic oncolytic HSV was constructed, in which both fusion mechanisms were incorporated into a single virus (Synco-2D). Evaluation of this virus through intraperitoneal administration on disseminated ovarian cancer led to 75% of mice tumor free.

In the data demonstrated herein, the singly and doubly fusogenic HSVs were directly compared for their ability to destroy a human prostate cancer cell line in vitro and the tumors established from this cell line in vivo. In vitro results show that Synco-2D has a significantly higher killing ability than Synco-2 on PC-3M-Pro4, indicating that the additional cell-membrane fusion mechanism contained in the doubly fusogenic virus has increased the tumor-destroying power of the virus. This notion was supported by the in vivo data that show that administration of Synco-2D produced the most dramatic therapeutic effect on lung metastasis of prostate cancer, a result that is significantly better than that from Synco-2 administration. However, the therapeutic effect on the orthotopic tumor from Synco-2D is only marginally better than that of Synco-2 after systemic administered of the viruses. The likely reasons for this differential effect on the tumor in primary site and the metastatic tumor are probably due to the discrepancy between the tumor size and amount of virus distribution after systemic delivery. By the time the viruses were administered, the orthotopic tumor volume was already bulky and was probably substantially bigger than the lung metastatic nodules. In addition, systemic administration of oncolytic HSVs can only distribute a limited amount of viruses to the tumor mass. Therefore, with only a limited amount of viruses distributed to a bulky tumor, even a more effective virus may not produce a noticeably better therapeutic effect than Synco-2. Thus, in embodiments wherein bulky solid tumors are treated, it is preferable to surgically remove at least part of the bulky tumors.

In contrary to the orthotopic tumors, by the time of oncolytic HSV administration, the lung metastatic tumor nodules were probably still quite small, as judging by the relatively small tumor size in the lung even by the endpoint of the experiment. In addition, due to rich blood supply and the filtering capability, the tumor masses in the lung may have a better virus distribution than the orthotopic tumor after systemic administration of the viruses. In this consideration, it is interesting to note that earlier studies by the present inventors demonstrated that administration of Fu-10 also produced a dramatically therapeutic effect on lung metastases of breast cancer (Fu and Zhang, 2002). Confirmation of virus distribution preference to lung may provide future guidance for treating either primary or metastatic tumors in this organ with these oncolytic agents.

Syncytia formation mediated by fusogenic glycoproteins relies on the initial binding of fusogenic glycoproteins with their specific receptors on target cells, which induces ordered structural changes of the membrane lipid bilayers, leading in turn to lipid mixing and eventual membrane fusion either between viral and cellular membranes or among cellular membranes (Lentz et al., 2000). The cellular receptor for GALV.fus has been identified as PiT1, a type III sodium-dependent phosphate transporter (Johann et al., 1993; Kavanaugh et al., 1994). However, the membrane fusion induced by HSV is more complex, requiring the participation of multiple viral glycoproteins and at least two specific cellular receptors on the cell surface. For HSV, several viral proteins and probably more than one cellular receptor are required for inducing cell—cell fusion include gB, gD, gH, and gL (Spear, 1993; Turner et al., 1998), and probably more than one Cellular receptor (Terry-Allison et al., 2001; 1998). Therefore, it is possible that, in addition to be quantitatively advantageous over the singly fusogenic oncolytic HSV, therapeutic administration of Synco-2D may reduce the emergence of therapy-resistant tumor cells. As tumor cells resistant to syncytia-formation mediated by one membrane-fusion mechanism due to receptor down-regulation or mutation will still be destroyed by syncytia formation resulting from another mechanism. The recent finding that Synco-2D infection but not Fu-10 or Synco-2 can cause syncytial formation on several-murian and one human tumor cell lines supports this possibility.

Several previous publications demonstrated that oncolytic HSVs could inhibit prostate tumor growth both in vitro and in vivo ((Walker et al., 1999; Taneja et al., 2001; Cozzi et al., 2002; Jorgensen et al., 2001)). However, most of these studies were conducted on subcutaneously established prostate cancer xenografts with intratumor injection of the viruses. It is critically important for new therapy strategies such as virotherapy for cancer to be effective against multifocal, diffuse disease, as is the present invention. The demonstration herein of significant therapeutic effect on lung metastases of prostate cancer after intravenous administration of Synco-2D at a moderate dose indicates that this doubly fusogenic oncolytic HSV is an effective therapeutic agent for destroying established human prostate tumor metastases.

Although the current protocol may be applicable to the treatment of clinical prostate cancer skeletal metastasis, some precautions need to be observed. Although oncolytic HSV is refrained to replicate only in tumor cells, the syncytial formation from these fusogenic oncolytic HSVs is potentially toxic to normal cells, and if uncontrolled, will pose a safety concern. This is particularly true when systemic administration is required such as this experimental design. During the construction of Synco-2D, several strategies were employed to restrict the syncytial formation to tumor tissues. First, a strict-late viral promoter was used to direct GALV.fus gene expression. The activity of this strict-late promoter, UL38p, has been shown to remain confined to the tumor tissue after systemic administration with an oncolytic HSV (Fu et al., 2002). Thus, GALV.fus-mediated syncytial formation was linked to the virus to replicate conditionally in tumor cells. Second, the syncytial formation from mutagenized HSV is mainly due to aberrant expression of several viral glycoproteins, such as gB and gK (Read et al., 1980; Bond et al., 1982; Pogue-GEile et al., 1984; Person et al., 1982). As these glycoproteins are encoded by late genes whose expression depends upon viral DNA replication, the cell membrane fusion mediated by this mechanism would only occur in tumor cells (where virus can undergo a full infection cycle) but not in normal nondividing cells (where virus replication is restricted and very low levels of glycoproteins are expressed). The demonstrations in previous studies that blocking viral DNA replication completely abolishes syncytia-forming ability of Fu-10 (the selected syncytial mutant from G207) (Fu and Zhang, 2002) and that Synco-2 (containing GALV.fus driven by UL38p) cannot induce syncytial formation in nondividing cells (Fu et al., 2002) strongly suggest that Synco-2D retains the safety profile of a conventional oncolytic HSV. More comprehensive toxicity studies on these fusogenic oncolytic HSVs in sensitive animals will further strength its potential at clinical application.

In summary, it is shown herein that an enhanced fusogenic HSV (1) facilitates cell-to-cell spread of virus particles; (2) supports the dispersion in a peritoneal cavity; and (3) is compatible with a replication-competent HSV system for the treatment of advanced cancer, such as ovarian cancer or prostate cancer.

### REFERENCES

The following references and others cited herein but not listed here, to the extent that they provide exemplary procedural or other details supplementary to those set forth herein, are specifically incorporated herein by reference.

### Publications

Advani, S. J., Sibley, G. S., Song, P. Y., Hallahan, D. E., Kataoka, Y., Roizman, B. and Weichselbaum, R. R. (1998). Gene Ther 5, 160-165.

Alemany, R., Gomez-Manzano, C., Balague, C., Yung, W. K., Curiel, D. T., Kyritsis, A. P. and Fueyo, J. (1999). Exp Cell Res 252, 1-12.

Al-Hendy A., Magliocco A. M., Al-Tweigeri T., Braileanu G., Crellin N., Li H., Strong T., Curiel D., and Chedrese P. J. (2000). Ovarian cancer gene therapy: repeated treatment with thymidine kinase in an adenovirus vector and ganciclovir improves survival in a novel immunocompetent murine model. Am. J. Obstet. Gynecol. 182: 553-559

Almendro N, Bellon T, Rius C, Lastres P, et al. Cloning of the human platelet endothelial cell adhesion molecule-1 promoter and its tissue-specific expression. Structural and functional characterization. J Immunol Dec. 15, 1996;157(12):5411-21.

Andreansky, S. S., B. He, G. Y. Gillespie, L. Soroceanu, J. Markert, J. Chou, B. Roizman and R. J. Whitley (1996). The application of genetically engineered herpes simplex viruses to the treatment of experimental brain tumors. Proc Natl Acad Sci USA. 93(21): 11313-8.

Andreansky, S., He, B., van Cott, J., McGhee, J., Markert, J. M., Gillespie, G. Y., Roizman, B. and Whitley, R. J. (1998). Gene Ther 5, 121.

Angel et al, Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Auzenne, E., Donato, N. J., Li, C., Leroux, E., Price, R. E., Farquhar, D., and Klostergaard, J. (2002). Superior therapeutic profile of poly-L-glutamic acid-paclitaxel copolymer compared with taxol in xenogeneic compartmental models of human ovarian carcinoma. Clin. Cancer Res. 8: 573-581

Babiss, L. E., Friedman, J. M. and Darnell, J. E. (1987). Cellular promoters incorporated into the adenovirus genome. Effect of viral DNA replication on endogenous and exogenous gene transcription. J. Mol. Biol. 193: 643-650.

Banerji et al, Cell, 27:299, 1981.

Banerji et al., Cell, 35:729, 1983.

Basu, S., R. J. Binder, R. Suto, K. M. Anderson and P. K. Srivastava (2000). Necrotic but not apoptotic cell death releases heat shock proteins, which deliver a partial maturation signal to dendritic cells and activate the NF-kappa B pathway. Int Immunol. 12(11): 1539-46.

Bateman, A., F. Bullough, S. Murphy, L. Emiliusen, D. Lavillette, F. L. Cosset, R. Cattaneo, S. J. Russell and R. G. Vile (2000). Fusogenic membrane glycoproteins as a novel class of genes for the local and immune-mediated control of tumor growth. Cancer Res. 60(6): 1492-7.

Bateman, A. R., K. J. Harrington, T. Kottke, A. Ahmed, A. A. Melcher, M. J. Gough, E. Linardakis, D. Riddle, A. Dietz, C. M. Lohse, S. Strome, T. Peterson, R. Simari and R. G. Vile (2002). Viral fusogenic membrane glycoproteins kill solid tumor cells by nonapoptotic mechanisms that promote cross presentation of tumor antigens by dendritic cells. Cancer Res. 62(22): 6566-78.

Berkhout et al., Cell, 59:273, 1989.

Bischoff, J. R., et al. (1996). An adenovirus mutant that replicates selectively in p53-deficient human tumor cells. Science. 274: 373-376.

Blanar et al, EMBO J, 8:1139, 1989.

Blank, S. V., Rubin, S. C., Coukos, G., Amin, K. M., Albelda, S. M. and Molnar-Kimber, K. L. (2002). Hum Gene Ther 13, 627-639.

Bodine and Ley, EMBO J, 6:2997, 1987.

Bond, V. C., Person, S. and Warner, S. C. (1982). J Gen Virol 61, 245.

Bookman M. A. (1998). Biological therapy of ovarian cancer: current directions. Semin. Oncol. 25: 381-396

Boon, T. and P. van der Bruggen (1996). Human tumor antigens recognized by T lymphocytes. J Exp Med. 183(3): 725-9.

Boshart et al., Cell, 41:521, 1985.

Bosze et al., EMBO J., 5:1615, 1986.

Braddock et al., Cell, 58:269, 1989.

Buick, R. N., Pullano, R., and Trent, J. M. (1985). Comparative properties of five human ovarian adenocarcinoma cell lines. Cancer Res. 45: 3668-3676

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Carroll, N. M., E. A. Chiocca, K. Takahashi and K. K. Tanabe (1996). Enhancement of gene therapy specificity for diffuse colon carcinoma liver metastases with recombinant herpes simplex virus. Ann Surg. 224(3): 323-9; discussion 329-30.

Celander and Haseltine, J. Virology, 61:269, 1987.

Celander et al., J. Virology, 62:1314, 1988.

Chahlavi A., Rabkin S., Todo T., Sundaresan P., and Martuza R. (1999). Effect of prior exposure to herpes simplex virus 1 on viral vector-mediated tumor therapy in immunocompetent mice. Gene Ther. 6: 1751-1758.

Chahlavi, A., Todo, T., Martuza, R. L. and Rabkin, S. D. (1999). Neoplasia 1, 162.

Chahlavi, A., S. Rabkin, T. Todo, P. Sundaresan and R. Martuza (1999). Effect of prior exposure to herpes simplex virus 1 on viral vector-mediated tumor therapy in immunocompetent mice. Gene Ther. 6(10): 1751-8.

Chandler et al., Cell, 33:489, 1983.

Chang et al., Mol. Cell. Biol., 9:2153, 1989.

Chase, M., Chung, R. Y. and Chiocca, E. A. (1998). Nat Biotechnol 16, 444-448.

Chatterjee et al., Proc. Nat'l Acad. Sci. USA., 86:9114, 1989.

Choi et al., Cell, 53:519, 1988.

Cohen et al., “A Repetitive Sequence Element 3′ of the human c-Ha-ras1 Gene Has Enhancer Activity,” J. Cell. Physiol., 5:75, 1987.

Cookson, M. M. (2001). Cancer Control 8, 133-140.

Costa et al., Mol. Cell. Biol., 8:81, 1988.

Coukos, G., A. Makrigiannakis, E. H. Kang, D. Caparelli, I. Benjamin, L. R. Kaiser, S. C. Rubin, S. M. Albelda and K. L. Molnar-Kimber (1999). Use of carrier cells to deliver a replication-selective herpes simplex virus-i mutant for the intraperitoneal therapy of epithelial ovarian cancer. Clin Cancer Res. 5(6): 1523-37.

Coukos, G., Makrigiannakis, A., Montas, S., Kaiser, L. R., Toyozumi, T., Benjamin, I., Albelda, S. M., Rubin, S. C., and Molnar-Kimber, K. L. (2000). Multi-attenuated herpes simplex virus-i mutant G207 exerts cytotoxicity against epithelial ovarian cancer but not normal mesothelium and is suitable for intraperitoneal oncolytic therapy. Cancer Gene Ther. 7: 275-28

Cozzi, P. J., Burke, P. B., Bhargav, A., Heston, W. D., Huryk, B., Scardino, P. T. and Fong, Y. (2002). Prostate 53, 95.

Cripe et al., EMBO J., 6:3745, 1987.

Culotta and Hamer, Mol. Cell. Biol., 9:1376, 1989.

Dandolo et al., J. Virology, 47:55, 1983.

De Villiers et al., Nature, 312:242, 1984.

Deschamps et al., Science, 230:1174, 1985.

Diaz, R. M., A. Bateman, L. Emiliusen, A. Fielding, D. Trono, S. J. Russell and R. G. Vile (2000). A lentiviral vector expressing a fusogenic glycoprotein for cancer gene therapy. Gene Ther. 7(19): 1656-63.

Diaz, R. M., Bateman, A., Emiliusen, L., Fielding, A., Trono, D., Russell, S. J., and Vile, R. G. (2000). A lentiviral vector expressing a fusogenic glycoprotein for cancer gene therapy. Gene Ther. 7: 1656-1663

Dranoff, G., E. Jaffee, A. Lazenby, P. Golumbek, H. Levitsky, K. Brose, V. Jackson, H. Hamada, D. Pardoll and R. C. Mulligan (1993). Vaccination with irradiated tumor cells engineered to secrete murine granulocyte-macrophage colony-stimulating factor stimulates potent, specific, and long-lasting anti-tumor immunity. Proc Natl Acad Sci U S A. 90(8): 3539-43.

Edbrooke et al., Mol. Cell. Biol., 9:1908, 1989.

Edlund et al., Science, 230:912, 1985.

Feng and Holland, Nature, 334:6178, 1988.

Fielding, A. K., Chapel-Fernandes, S., Chadwick, M. P., Bullough, F. J., Cosset, F. L., and Russell, S. J. (2000). A hyperfusogenic gibbon ape leukemia envelope glycoprotein: targeting of a cytotoxic gene by ligand display. Hum. Gene Ther. 11: 817-826

Fielding, A. K., S. Chapel-Fernandes, M. P. Chadwick, F. J. Bullough, F. L. Cosset and S. J. Russell (2000). A hyperfusogenic gibbon ape leukemia envelope glycoprotein: targeting of a cytotoxic gene by ligand display. Hum Gene Ther. 11(6): 817-26.

Firak and Subramanian, Mol. Cell. Biol., 6:3667, 1986.

Flanagan, W. M., et al. (1991). Analysis of the herpes simplex virus type 1 promoter controlling the expression of UL38, a true late gene involved in capsid assembly. J. Virol. 65: 769-786.

Foecking and Hofstetter, “Powerful and/or Versatile Enhancer-Promoter Unit for [mammalian, plant, fungus, bacteria?] Expression Vectors,” Gene, 45:101, 1986.

Fu, X. and Zhang, X. (2002). Potent systemic antitumor activity from an oncolytic herpes simplex virus of syncytial phenotype. Cancer Res. 62: 2306-2312.

Fu, X., and Zhang, X. (2002). Potent systemic antitumor activity from an oncolytic herpes simplex virus of syncytial phenotype. Cancer Res. 62: 2306-2312

Fu, X., Tao, L., Jin, A., Vile, R. G., Brenner, M. K., and Zhang, X (2002). Expression of a fusogenic membrane glycoprotein by an oncolytic herpes simplex virus potentiates the viral antitumor effect. Cancer Res. 62: in press

Fu, X., Zhang, X. (2000). Delivery of herpes simplex virus vectors through liposome formulation. Mol. Ther. 4: 447-453

Fu, X., Tao, L., Jin, A., Vile, R., Brenner, M. and Zhang, X. (2002). Nat. Med., In press.

Fu, X., Meng, F., Tao, L., Jin, A. and Zhang, X. (2002). Hum. Gene Ther., In press.

Fujita et al., Cell, 49:357, 1987.

Gilles et al., Cell, 33:717, 1983.

Gloss et al., EMBO J., 6:3735, 1987.

Godbout et al., Mol. Cell. Biol., 8:1169, 1988.

Goodart, S. A., Guzowski, J. F., Rice, M. K. and Wagner, E. K. (1992). Effect of genomic location on expression of beta-galactosidase mRNA controlled by the herpes simplex virus type 1 UL38 promoter. J. Virol. 66: 2973-2981.

Goodboum and Maniatis, Proc. Nat'l Acad. Sci. USA, 85:1447, 1988.

Goodboum et al., Cell, 45:601, 1986.

Greene et al., Immunology Today, 10:272, 1989.

Greenlee, R. T., Hill-Harmon, M. B., Murray, T., Thun M. (2001). Cancer statistics, 2001. CA Cancer J. Clin. 51: 15-36

Grosschedl and Baltimore, Cell, 41:885, 1985.

Guzowski, J. F. and Wagner, E. K. (1993). Mutational analysis of the herpes simplex virus type 1 strict late UL38 promoter/leader reveals two regions critical in transcriptional regulation. J. Virol. 67: 5098-5108.

Guzowski, J. F., and Wagner, E. K. (1993). Mutational analysis of the herpes simplex virus type 1 restrict late UL38 promoter/leader reveals two regions critical in transcriptional regulation. J. Virol. 67: 5098-5108

Guzowski, J. F., Singh, J. and Wagner, E. K. (1994). Transcriptional activation of the herpes simplex virus type 1 UL38 promoter conferred by the cis-acting downstream activation sequence is mediated by a cellular transcription factor. J. Virol. 68: 7774-7789.

Haslinger and Karin, Proc. Nat'l Acad. Sci. USA., 82:8572, 1985.

Hauber and Cullen, J. Virology, 62:673, 1988.

Heise, C. C., A. Williams, J. Olesch and D. H. Kim (1999). Efficacy of a replication-competent adenovirus (ONYX-015) following intratumoral injection: intratumoral spread and distribution effects. Cancer Gene Ther. 6(6): 499-504.

Heise, C., Ganly, I., Kim, Y. T., Sampson-Johannes, A., Brown, R., and Kim, D. (2000). Efficacy of a replication-selective adenovirus against ovarian carcinomatosis is dependent on tumor burden, viral replication and p53 status. Gene Ther. 7: 1925-1929

Hen et al., Nature, 321:249, 1986.

Hensel et al., Lymphokine Res., 8:347, 1989.

Hermiston, T. (2000). J Clin Invest 105, 1169-1172.

Herr and Clarke, Cell, 45:461, 1986.

Herrlinger, U., et al. (2000). HSV-1 infected cell proteins influence tetracycline-regulated transgene expression. J Gene Med. 2: 379-389.

Higuchi, H., Bronk, S. F., Bateman, A., Harrington, K., Vile, R. G., Gores, G. J. (2000). Viral fusogenic membrane glycoprotein expression causes syncytia formation with bioenergetic cell death: implications for gene therapy. Cancer Res. 60: 6396-6402

Higuchi, H., S. F. Bronk, A. Bateman, K. Harrington, R. G. Vile and G. J. Gores (2000). Viral fusogenic membrane glycoprotein expression causes syncytia formation with bioenergetic cell death: implications for gene therapy. Cancer Res. 60(22): 6396-402.

Hirasawa, K., Nishikawa, S. G., Norman, K. L., Alain, T., Kossakowska, A., and Lee P. W. (2002). Oncolytic reovirus against ovarian and colon cancer. Cancer Res. 62: 1696-1701

Hirochika et al., J. Virol., 61:2599, 1987.

Hirsch et al., Mol. Cell. Biol., 10: 1959, 1990.

Holbrook et al., Virology, 157:211, 1987.

Holland, L. E., Anderson, K. P., Shipman, C., Jr. and Wagner, E. K. (1980). Viral DNA synthesis is required for the efficient expression of specific herpes simplex virus type 1 mRNA species. Virology. 101: 10-24.

Horlick and Benfield, Mol. Cell. Biol., 9:2396, 1989.

Huang et al., Cell, 27:245, 1981.

Huard, J., et al. (1996). The basal lamina is a physical barrier to herpes simplex virus-mediated gene delivery to mature muscle fibers. J. Virol. 70: 8117-8123.Huber, B. E., Richards, C. A. and Krenitsky, T. A. (1991). Retroviral-mediated gene therapy for the treatment of hepatocellular carcinoma: an innovative approach for cancer therapy. Proc Natl Acad Sci USA. 88: 8039-8043.

Huard, J., Goins, W. F. and Glorioso, J. C. (1995). Herpes simplex virus type 1 vector mediated gene transfer to muscle. Gene Ther. 2: 385-392.

Hug H, Costas M, Staeheli P, Aebi M, et al. Organization of the murine Mx gene and characterization of its interferon- and virus-inducible promoter. Mol Cell Biol August 1988;8(8):3065-79.

Hull, G. W., McCurdy, M. A., Nasu, Y., Bangma, C. H., Yang, G., Shimura, S., Lee, H. M., Wang, J., Albani, J., Ebara, S., Sato, T., Timme, T. L. and Thompson, T. C. (2000). Clin Cancer Res 6, 4101-4109.

Hwang et al., Mol. Cell. Biol., 10:585, 1990.

Hwu, P., Freedman, R. S. (2002). The immnunotherapy of patients with ovarian cancer. J. Immunother. 25: 189-201

Imagawa et al., Cell, 51:251, 1987.

Imbra and Karin, Nature, 323:555, 1986.

Imler et al., Mol. Cell. Biol., 7:2558, 1987.

Jakobovits et al., Mol. Cell. Biol., 8:2555, 1988.

Jameel and Siddiqui, Mol. Cell. Biol., 6:710, 1986.

Januszeski, M. M., P. M. Cannon, D. Chen, Y. Rozenberg and W. F. Anderson (1997). Functional analysis of the cytoplasmic tail of Moloney murine leukemia virus envelope protein. J. Virol. 71(5): 3613-9.

Jaynes et al., Mol. Cell. Biol., 8:62, 1988.

Johann, S. V., van Zeijl, M., Cekleniak, J. and O'Hara, B. (1993). J Virol 67, 6733.

Johnson et al., Mol. Cell. Biol!, 9:3393, 1989.

Johnson, P. A. and Everett, R. D. (1986). DNA replication is required for abundant expression of a plasmid-borne late US11 gene of herpes simplex virus type 1. Nucleic Acids Res. 14: 3609-3625.

Jorgensen, T. J., Katz, S., Wittmack, E. K., Varghese, S., Todo, T., Rabkin, S. D. and Martuza, R. L. (2001). Neoplasia 3, 451.

Kadesch and Berg, Mol. Cell. Biol., 6:2593, 1986.

Karin et al., Mol. Cell. Biol., 7:606, 1987.

Katinka et al., Cell, 20:393, 1980.

Katinka et al., Nature, 290:720, 1981.

Kavanaugh, M. P., Miller, D. G., Zhang, W., Law, W., Kozak, S. L., Kabat, D. and Miller, A. D. (1994). Proc Natl Acad Sci USA 91, 7071.

Kawamoto et al., Mol. Cell. Biol., 8:267, 1988.

Kaye, S. B., Eisenhauer, E. A., and Hamilton, T. C. (1999). New non-cytotoxic approaches to ovarian cancer. Ann. Oncol. 10: 65-68

Kiledjian et al., Mol. Cell. Biol., 8:145, 1988.

Kim, D. (2001). Expert Opin Biol Ther 1, 525-538.

Klamut et al., Mol. Cell. Biol., 10:193, 1990.

Koch et al., Mol. Cell. Biol., 9:303, 1989.

Koga, S., et al. (2000). A novel telomerase-specific gene therapy: gene transfer of caspase-8 utilizing the human telomerase catalytic subunit gene promoter. Hum Gene Ther. 11: 1397-1406.

Kramm, C. M., Chase, M., Herrlinger, U., Jacobs, A., Pechan, P. A., Rainov, N. G., Sena-Esteves, M., Aghi, M., Barnett, F. H., Chiocca, E. A., and Breakefield X. O. (1997). Therapeutic efficiency and safety of a second-generation replication-conditional HSV1 vector for brain tumor gene therapy. Hum. Gene Ther. 8: 2057-2068

Kramm, C. M., M. Chase, U. Herrlinger, A. Jacobs, P. A. Pechan, N. G. Rainov, M. Sena-Esteves, M. Aghi, F. H. Barnett, E. A. Chiocca and X. O. Breakefield (1997). Therapeutic efficiency and safety of a second-generation replication-conditional HSVI vector for brain tumor gene therapy. Hum Gene Ther. 8(17): 2057-68.

Kraus J, Woltje M, Schonwetter N, Hollt V. Alternative promoter usage and tissue specific expression of the mouse somatostatin receptor 2 gene. FEBS Lett May 29, 1998;428(3): 165-70.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunz et al., Nucl. Acids Res., 17:1121, 1989.

Lambright E. S., Kang E. H., Force S., Lanuti M., Caparrelli D., Kaiser L. R., Albelda S. M., and Molnar-Kimber K. L. (2000). Effect of preexisting anti-herpes immunity on the efficacy of herpes simplex viral therapy in a murine intraperitoneal tumor model. Mol. Ther. 2: 387-393

Lareyre J J, Thomas T Z, Zheng W L, Kasper S, et al. A 5-kilobase pair promoter fragment of the murine epididymal retinoic acid-binding protein gene drives the tissue-specific, cell-specific, and androgen-regulated expression of a foreign gene in the epididymis of transgenic mice. J Biol Chem Mar. 19, 1999;274(12):8282-90.

Larsen et al., Proc. Nat'l Acad. Sci. USA., 83:8283, 1986.

Laspia et al., Cell, 59:283, 1989.

Latham, J. P., Searle, P. F., Mautner, V. and James, N. D. (2000). Prostate-specific antigen promoter/enhancer driven gene therapy for prostate cancer: construction and testing of a tissue-specific adenovirus vector. Cancer Res. 60: 334-341.

Latimer et al., Mol. Cell. Biol., 10:760, 1990.

Lee et al., Mol. Endocrinol., 2: 404-411, 1988.

Lee et al., Nature, 294:228, 1981.

Lee S H, Wang W, Yajima S, Jose P A, et al. Tissue-specific promoter usage in the DIA dopamine receptor gene in brain and kidney. DNA Cell Biol November 1997;16(11):1267-75.

Lentz, B. R., Malinin, V., Haque, M. E. and Evans, K. (2000). Curr Opin Struct Biol 10, 607./Levinson et al., Nature, 295:79, 1982.

Li, H., Y. S. Haviv, C. A. Derdeyn, J. Lam, C. Coolidge, E. Hunter, D. T. Curiel and J. L. Blackwell (2001). Human immunodeficiency virus type 1-mediated syncytium formation is compatible with adenovirus replication and facilitates efficient dispersion of viral gene products and de novo-synthesized virus particles. Hum Gene Ther. 12(18): 2155-65.

Lin et al., Mol. Cell. Biol., 10:850, 1990.

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Mabjeesh, N. J., Zhong, H. and Simons, J. W. (2002). Endocr Relat Cancer 9, 115-139.

MacKie, R. M., Stewart, B. and Brown, S. M. (2001). Lancet 357, 525-526.

Majors and Varmus, Proc. Nat'l Acad. Sci. USA., 80:5866, 1983.

Markert, J. M., M. D. Medlock, S. D. Rabkin, G. Y. Gillespie, T. Todo, W. D. Hunter, C. A. Palmer, F. Feigenbaum, C. Tornatore, F. Tufaro and R. L. Martuza (2000). Conditionally replicating herpes simplex virus mutant, G207 for the treatment of malignant glioma: results of a phase I trial. Gene Ther. 7(10): 867-74.

Markert, J. M., Medlock, M. D., Rabkin, S. D., Gillespie, G. Y., Todo, T., Hunter, W. D., Palmer, C. A., Feigenbaum, F., Tornatore, C., Tufaro, F., and Martuza, R. L. (2000). Conditionally replicating herpes simplex virus mutant, G207 for the treatment of malignant glioma: results of a phase I trial. Gene Ther. 7: 867-874

Martuza, R. L., Malick, A., Markert, J. M., Ruffner, K. L. and Coen, D. M. (1991). Experimental therapy of human glioma by means of a genetically engineered virus mutant. Science. 252: 854-856.

Martuza, R. L. (1997). Nat Med 3, 1323.

Matzinger, P. (2002). The danger model: a renewed sense of self. Science. 296(5566): 301-5.

McGuire, W. P., Hoskins, W. J., Brady, M. F., Kucera, P. R., Partridge, E. E., Look, K. Y., Clarke-Pearson, D. L., and Davidson, M. (1996). Cyclophosphamide and cisplatin compared with paclitaxel and cisplatin in patients with stage III and stage IV ovarian cancer. N. Engl. J. Med. 334: 1-6

McNeall et al., Gene, 76:81, 1989.

Melcher, A., Tordryk, S., Hardwick, N., Ford, M., Jacobson, M., and Vile, R. G. (1998). Tumor immunogenecity is determined by the mechanism of cell death via induction og heat shock protein expression. Nat. Med. 4: 581-587

Melcher, A., M. Gough, S. Todryk and R. Vile (1999). Apoptosis or necrosis for tumor immunotherapy: what's in a name? J Mol Med. 77(12): 824-33.

Miksicek et al., Cell, 46:203, 1986.

Mineta, T., S. D. Rabkin, T. Yazaki, W. D. Hunter and R. L. Martuza (1995). Attenuated multi-mutated herpes simplex virus-1 for the treatment of malignant gliomas. Nat Med. 1(9): 938-43.

Mordacq and Linzer, Genes and Dev., 3:760, 1989.

Moreau et al., Nucl. Acids Res., 9:6047, 1981.

Muesing et al., Cell, 48:691, 1987.

Nemunaitis J., Ganly I., Khuri F., Arseneau J., Kuhn J., McCarty T., Landers S., Maples P., Romel L., Randlev B., Reid T., Kaye S., and Kim D. (2000). Selective replication and oncolysis in p53 mutant tumors with ONYX-015, an E1B-55 kD gene-deleted adenovirus, in patients with advanced head and neck cancer: a Phase II trial. Cancer Res. 60: 6359-6366

Nemunaitis, J., F. Khuri, I. Ganly, J. Arseneau, M. Posner, E. Vokes, J. Kuhn, T. McCarty, S.

Landers, A. Blackburn, L. Romel, B. Randlev, S. Kaye and D. Kim (2001). Phase II trial of intratumoral administration of ONYX-015, a replication-selective adenovirus, in patients with refractory head and neck cancer. J Clin Oncol. 19(2): 289-98.

Ng et al., Nuc. Acids Res., 17:601, 1989.

NIH Consensus Conference. Ovarian cancer. Screening, treatment, and follow-up. NIH Consensus Development Panel on Ovarian Cancer. (1995). J. Am. Med. Assoc. 273: 491-497

Nomoto S, Tatematsu Y, Takahashi T, Osada H. Cloning and characterization of the alternative promoter regions of the human LIMK2 gene responsible for alternative transcripts with tissue-specific expression. Gene Aug. 20, 1999;236(2):259-71.

Ondek et al., EMBO J., 6:1017, 1987.

Ornitz et al., Mol. Cell. Biol., 7:3466, 1987.

Ozols, R. F. (2000). Management of advanced ovarian cancer consensus summary. Semin. Oncol. 27: 47-49

Palmiter et al., Nature, 300:611, 1982.

Pawlik, T. M., H. Nakamura, S. S. Yoon, J. T. Mullen, S. Chandrasekhar, E. A. Chiocca and K. K. Tanabe (2000). Oncolysis of diffuse hepatocellular carcinoma by intravascular administration of a replication-competent, genetically engineered herpesvirus. Cancer Res. 60(11): 2790-5.

Parker, J. N., Gillespie, G. Y., Love, C. E., Randall, S., Whitley, R. J. and Markert, J. M. (2000). Proc Natl Acad Sci USA 97, 2208.

Pech et al., Mol. Cell. Biol., 9:396, 1989.

Peng, K-W., TenEyck, C. J., Galanis, E., Kalli, K. R., Hartmann, L. C. and Russel, S. J. (2002). Intraperitoneal therapy of ovarian cancer using an engineered measles virus. Cancer Res. 62: 4656-4662

Pennisi, E. (1996). Science 274, 342-343.

Perez-Stable and Constantini, Mol. Cell. Biol., 10:1116, 1990.

Person, S., Kousoulas, K. G., Knowles, R. W., Read, G. S., Holland, T. C., Keller, P. M. and Warner, S. C. (1982). Virology 117, 293.

Pettaway, C. A., Pathak, S., Greene, G., Ramirez, E., Wilson, M. R., Killion, J. J. and Fidler, I. J. (1996). Clin Cancer Res 2, 1627-1636.

Picard and Schaffner, Nature, 307:83, 1984.

Pinkert et al., Genes and Dev., 1:268, 1987.

Pogue-Geile, K. L., Lee, G. T., Shapira, S. K. and Spear, P. G. (1984). Virology 136, 100-109.

Ponta et al., Proc. Nat'l Acad. Sci. USA., 82:1020, 1985.

Porton et al., Mol. Cell. Biol., 10:1076, 1990.

Queen and Baltimore, Cell, 35:741, 1983.

Quinn et al., Mol. Cell. Biol., 9:4713, 1989.

Rampling, R., G. Cruickshank, V. Papanastassiou, J. Nicoll, D. Hadley, D. Brennan, R. Petty, A. MacLean, J. Harland, E. McKie, R. Mabbs and M. Brown (2000). Toxicity evaluation of replication-competent herpes simplex virus (ICP 34.5 null mutant 1716) in patients with recurrent malignant glioma. Gene Ther. 7(10): 859-66.

Read, G. S., Person, S. and Keller, P. M. (1980). J Virol 35, 105.

Redondo et al., Science, 247:1225, 1990.

Reisman and Rotter, Mol. Cell. Biol., 9:3571, 1989.

Resendez Jr. et al., Mol. Cell. Biol., 8:4579, 1988.

Ripe et al., Mol. Cell. Biol., 9:2224, 1989.

Rittling et al., Nucl. Acids Res., 17:1619, 1989.

Robertson, M. W., III, Barnes, M. N., Rancourt, C., Wang, M., Grim, J., Alvarez, R. D., Siegal, G. P., and Curiel, D. T. (1998). Gene therapy for ovarian carcinoma. Semin. Oncol. 25: 397-406

Rosen et al., Cell, 41:813, 1988.

Rubin, S. C., Randall, T. C., Armstrong, K. A., Chi, D. S., and Hoskins, W. J. (1999). Ten-year follow-up of ovarian patients after second-look laparotomy with negative findings. Obstet. Gynecol. 93: 21-24

Saeki, Y., Ichikawa, T., Saeki, A., Chiocca, E. A., Tobler, K., Ackermann, M., Breakefield, X. O., and Fraefel, C. (1998). Herpes simplex virus type 1 DNA amplified as bacterial artificial chromosome in Escherichia coli: rescue of replication-competent virus progeny and packing of amplicon vectors. Hum. Gene Ther. 9: 2787-2794

Saeki, Y., T. Ichikawa, A. Saeki, E. A. Chiocca, K. Tobler, M. Ackermann, X. O. Breakefield and C. Fraefel (1998). Herpes simplex virus type 1 DNA amplified as bacterial artificial chromosome in Escherichia coli: rescue of replication-competent virus progeny and packaging of amplicon vectors. Hum Gene Ther. 9(18): 2787-94.

Satake et al., “Biological Activities of Oligonucleotides Spanning the F9 Point Mutation Within the Enhancer Region of Polyoma Virus DNA,” J. Virology, 62:970, 1988.

Schaffer, P. A., Aron, G. M., Biswal, N., and Benyesh-Melnick M. (1973). Temperature-sensitive mutants of herpes simplex virus type 1: isolation, complementation and partial characterization. Virology. 52: 57-71

Schaffner et al., J. Mol. Biol., 201:81, 1988.

Schadendorf, D., A. Paschen and Y. Sun (2000). Autologous, allogeneic tumor cells or genetically engineered cells as cancer vaccine against melanoma. Immunol Lett. 74(1): 67-74.

Schang, L. M., J. Phillips and P. A. Schaffer (1998). Requirement for cellular cyclin-dependent kinases in herpes simplex virus replication and transcription. J. Virol. 72(7): 5626-37.

Schellingerhout, D., et al. (1998). Mapping the in vivo distribution of herpes simplex virions. Hum Gene Ther. 9: 1543-1549.

Schuur, E. R., et al. (1996). Prostate-specific antigen expression is regulated by an upstream enhancer. J. Biol. Chem. 271: 7043-7051.

Searle et al., Mol. Cell. Biol., 5:1480, 1985.

Selby, P. J., Thomas, J. M., Monaghan, P., Sloane, J., and Peckman, M. J. (1980). Human tumor xenografts established and serially transplanted in mice immnulogically derived by thymectomy, cytosine arabinoside and whole-body irradiation. Br. J. Cancer. 41: 52-61

Sharp and Marciniak, Cell, 59:229, 1989.

Shaul and Ben-Levy, EMBO J., 6:1913, 1987.

Sherman et al., Mol. Cell. Biol., 9:50, 1989.

Shevrin, D. H., Gomy, K. I. and Kukreja, S. C. (1989). Prostate 15, 187-194.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Spear, P. G. (1993) in Membrane fusion induced by herpes simplex virus, eds. J. Bentz, (CRC Press: Boca Raton), pp. 201.

Spear, M. A., Sun, F., Eling, D. J., Gilpin, E., Kipps, T. J., Chiocca, E. A. and Bouvet, M. (2000). Cancer Gene Ther 7, 1051

Stephens and Hentschel, Biochem. J, 248:1, 1987.

Stephenson, R. A., Dinney, C. P., Gohji, K., Ordonez, N. G., Killion, J. J. and Fidler, I. J. (1992). J Natl Cancer Inst 84, 951-957.

Stuart et al., Nature, 317:828, 1985.

Sullivan and Peterlin, Mol. Cell. Biol., 7:3315, 1987.

Sundaresan, P., W. D. Hunter, R. L. Martuza and S. D. Rabkin (2000). Attenuated, replication-competent herpes simplex virus type 1 mutant G207: safety evaluation in mice. J. Virol. 74(8): 3832-41.

Swartzendruber and Lehman, J. Cell. Physiology, 85:179, 1975.

Takebe et al., Mol. Cell. Biol., 8:466, 1988.

Tamura, Y., Peng, P., Liu, K., Daou, M., and Srivastava, P. K. (1997). Immunotherapy of tumors with autologous tumor-derived heat shock protein preparations. Science. 278: 117-120

Tanaka, S., et al. (2000). Targeted killing of carcinoembryonic antigen (CEA)-producing cholangiocarcinoma cells by polyamidoamine dendrimer-mediated transfer of an Epstein-Barr virus (EBV)-based plasmid vector carrying the CEA promoter. Cancer Gene Ther. 7: 1241-1250.

Taneja, S., MacGregor, J., Markus, S., Ha, S. and Mohr, I. (2001). Proc Natl Acad Sci USA 98, 8804.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Terry-Allison, T., Montgomery, R. I., Warner, M. S., Geraghty, R. J. and Spear, P. G. (2001). Virus Res 74, 39-45.

Terry-Allison, T., Montgomery, R. I., Whitbeck, J. C., Xu, R., Cohen, G. H., Eisenberg, R. J. and Spear, P. G. (1998). J Virol 72, 5802-5810.

Thiesen et al., J. Virology, 62:614, 1988.

Toda, M., S. D. Rabkin and R. L. Martuza (1998). Treatment of human breast cancer in a brain metastatic model by G207, a replication-competent multimutated herpes simplex virus 1. Hum Gene Ther. 9(15): 2177-85.

Todo, T., F. Feigenbaum, S. D. Rabkin, F. Lakeman, J. T. Newsome, P. A. Johnson, E. Mitchell, D. Belliveau, J. M. Ostrove and R. L. Martuza (2000). Viral shedding and biodistribution of G207, a multimutated, conditionally replicating herpes simplex virus type 1, after intracerebral inoculation in aotus. Mol Ther. 2(6): 588-95.

Todo, T., Martuza, R. L., Dallman, M. J. and Rabkin, S. D. (2001). Cancer Res 61, 153.

Todo, T., S. D. Rabkin and R. L. Martuza (2000). Evaluation of ganciclovir-mediated enhancement of the antitumoral effect in oncolytic, multimutated herpes simplex virus type 1 (G207) therapy of brain tumors. Cancer Gene Ther. 7(6): 939-46.

Todryk, S., et al. (1999). Disabled infectious single-cycle herpes simplex virus as an oncolytic vector for immunotherapy of colorectal cancer. Hum Gene Ther. 10: 2757-2768.

Toyoizumi, T., R. Mick, A. E. Abbas, E. H. Kang, L. R. Kaiser and K. L. Molnar-Kimber (1999). Combined therapy with chemotherapeutic agents and herpes simplex virus type 1 ICP34.5 mutant (HSV-1716) in human non-small cell lung cancer. Hum Gene Ther. 10(18): 3013-29.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsumaki N, Kimura T, Tanaka K, Kimura J H, et al. Modular arrangement of cartilage- and neural tissue-specific cis-elements in the mouse alpha2(XI) collagen promoter. J Biol Chem Sep. 4, 1998;273(36):22861-4.

Turner, A., Bruun, B., Minson, T. and Browne, H. (1998). J Virol 72, 873.

Tyndall et al., Nuc. Acids. Res., 9:6231, 1981.

Vannice and Levinson, J. Virology, 62:1305, 1988.

Vasseur et al., Proc. Nat'l Acad. Sci. USA., 77:1068, 1980.

Vile, R. G. and Hart, I. R. (1993). Use of tissue-specific expression of the herpes simplex virus thymidine kinase gene to inhibit growth of established murine melanomas following direct intratumoral injection of DNA. Cancer Res. 53: 3860-3864.

Wagner, E. K., Guzowski, J. F. and Singh, J. (1995). Transcription of the herpes simplex virus genome during productive and latent infection. Prog Nucleic Acid Res Mol Biol. 51: 123-165.

Walker, J. R., K. G. McGeagh, P. Sundaresan, T. J. Jorgensen, S. D. Rabkin and R. L. Martuza (1999). Local and systemic therapy of human prostate adenocarcinoma with the conditionally replicating herpes simplex virus vector G207. Hum Gene Ther. 10(13): 2237-43.

Wang and Calame, Cell, 47:241, 1986.

Weber et al., Cell, 36:983, 1984.

Weinberger et al. Mol. Cell. Biol., 8:988, 1984.

Winoto and Baltimore, Cell, 59:649, 1989.

Wood, M., et al. (1999). Biodistribution of an adenoviral vector carrying the luciferase reporter gene following intravesical or intravenous administration to a mouse. Cancer Gene Ther. 6: 367-372.

Wu H K, Squire J A, Song Q, Weksberg R. Promoter-dependent tissue-specific expressive nature of imprinting gene, insulin-like growth factor II, in human tissues. Biochem Biophys Res Commun Apr. 7, 1997;233(1):221-6.

Yoon, S. S., Carroll, N. M., Chiocca, E. A. and Tanabe, K. K. (1998). Ann Surg 228, 366.

Yoon, S. S., H. Nakamura, N. M. Carroll, B. P. Bode, E. A. Chiocca and K. K. Tanabe (2000). An oncolytic herpes simplex virus type 1 selectively destroys diffuse liver metastases from colon carcinoma. Faseb J. 14(2): 301-11

Yutzey et al. “An Internal Regulatory Element Controls Troponin I Gene Expression,” Mol. Cell. Biol., 9:1397, 1989.

Yutzey et al. Mol. Cell. Biol., 9:1397, 1989.

Zhang, X., Efstathiou, S. and Simmons, A. (1994). Virology 202, 530-539.

Zhao-Emonet J C, Boyer O, Cohen J L, Klatzmann D. Deletional and mutational analyses of the human CD4 gene promoter: characterization of a minimal tissue-specific promoter. Biochim Biophys Acta Nov. 8, 1998;1442(2-3):109-19.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. A composition, comprising:
a vector comprising:
a first cell membrane fusion-generating activity; and
a second cell membrane fusion-generating activity.

2. The composition of claim 1, wherein said vector is a Herpes Simplex Virus vector.

3. The composition of claim 2, wherein the HSV vector is conditionally replicating.

4. The composition of claim 3, wherein conditionally replicating is defined as the vector comprising a strict late viral promoter.

5. The composition of claim 1, wherein the first cell membrane fusion-generating activity, the second cell membrane fusion-generating activity, or both comprise a mutation, said mutation conferring said cell membrane fusion-generating activity to the vector or a gene product encoded thereby.

6. The composition of claim 1, wherein the first cell membrane fusion-generating activity, the second cell membrane fusion-generating activity, or both comprise a nucleic acid sequence that encodes a fusogenic polypeptide.

7. The composition of claim 6, wherein the fusogenic polypeptide is further defined as a membrane glycoprotein.

8. The composition of claim 7, wherein the membrane glycoprotein is paramyxovirus F protein, HIV gp160 protein, SIV gp160 protein, retroviral Env protein, Ebola virus Gp, or the influenza virus haemagglutinin.

9. The composition of claim 7, wherein the glycoprotein is a membrane glycoprotein from gibbon ape leukemia virus (GALV).

10. The composition of claim 7, wherein the glycoprotein is a C-terminally truncated form of the gibbon ape leukemia virus envelope glycoprotein (GALV.fus).

11. The composition of claim 6, wherein the expression of the nucleic acid sequence is controlled by a strict late viral promoter.

12. The composition of claim 11, wherein the strict late viral promoter is the promoter of UL38 or Us11 of HSV.

13. The composition of claim 1, further comprising a pharmaceutically acceptable excipient.

14. A method of generating fusion between a first cell and a second cell, comprising the step of fusing the second cell membrane with the first cell membrane by introducing to the first cell a vector comprising a first cell membrane fusion-generating activity and a second cell membrane fusion-generating activity.

15. The method of claim 14, wherein the first cell, second cell, or both first and second cells are malignant cells.

16. The method of claim 15, wherein the malignant cells are in a solid tumor.

17. The method of claim 15, wherein the malignant cells are in a human.

18. The method of claim 17, wherein the introducing step is further defined as delivering the vector to the human.

19. The method of claim 18, wherein the delivering step is further defined as systemically delivering the vector to the human.

20. The method of claim 19, wherein the systemic delivery to the human is further defined as intravenously delivering the vector to the human.

21. The method of claim 14, wherein the step is repeated with a plurality of cells.

22. The method of claim 14, wherein the vector is a conditionally replicating Herpes Simplex Virus vector.

23. The method of claim 14, wherein the first cell membrane fusion-generating activity, the second cell membrane fusion-generating activity, or both comprise a mutation, said mutation conferring said cell membrane fusion-generating activity to the vector or a gene product encoded thereby.

24. The method of claim 14, wherein the first cell membrane fusion-generating activity, the second cell membrane fusion-generating activity, or both comprise a nucleic acid sequence that encodes a fusogenic polypeptide.

25. The method of claim 24, wherein the expression of the nucleic acid sequence is regulated by a strict late viral promoter.

26. The method of claim 25, wherein the strict late viral promoter is the promoter of UL38 or Us11 of HSV.

27. The method of claim 17, wherein the method further comprises the step of providing enhanced tumor antigen presentation compared to in the absence of said vector.

28. The method of claim 27, wherein said enhanced tumor antigen presentation provides an improved antitumor immunity compared to in the absence of said enhanced tumor antigen presentation.

29. A method of destroying a malignant cell, comprising the step of introducing to the cell a vector comprising a first cell membrane fusion-generating activity; and a second cell membrane fusion-generating activity, wherein following said introduction the membrane of the malignant cell fuses with another cell membrane.

30. The method of claim 27, wherein the malignant cell is in a human.

31. The method of claim 28, wherein the introduction step is further defined as administering at least about 1×10⁹ plaque forming units (pfu) of the vector to the human.

32. The method of claim 28, wherein the method further comprises administering an additional cancer therapy to the human.

33. The method of claim 30, wherein the additional cancer therapy is chemotherapy, radiation, surgery, immunotherapy, gene therapy, or a combination thereof.

34. The method of claim 30, wherein the method further comprises the step of providing enhanced tumor antigen presentation compared to in the absence of said vector.

35. The method of claim 34, wherein said enhanced tumor antigen presentation provides an improved antitumor immunity compared to in the absence of said enhanced tumor antigen presentation.

36. A composition comprising an oncolytic virus, wherein the virus comprises a strict late viral promoter.

37. The composition of claim 32, wherein said virus is further defined as being tumor-specific.

38. A method of generating a cell membrane fusion-generating Herpes Simplex Virus vector comprising the steps of:
introducing a mutation to a non-cell membrane fusion-generating Herpes Simplex Virus vector, said mutation conferring cell-membrane fusion-generating activity to the vector or a gene product encoded thereby; and
incorporating into said vector a nucleic acid sequence encoding a cell membrane fusion-generating polypeptide.

39. A composition, comprising:
a Herpes Simplex Virus vector comprising a mutation that confers to the vector or a gene product encoded thereby a cell membrane fusion-generating activity; and
a nucleic acid sequence encoding GALV.fus.

40. A method of destroying a malignant cell comprising introducing to said cell a composition comprising an oncolytic virus, wherein the virus comprises a strict late viral promoter.

41. A mammalian cell comprising the composition of claim 1.

42. A mammalian cell comprising the composition of claim 35.

43. A vector, comprising a first cell membrane fusion-generating activity and a second cell membrane fusion-generating activity, wherein said vector is obtainable by a method comprising at least one of the following steps:
generating a mutation in a nucleic acid sequence of the vector, wherein the mutation confers to the vector or a gene product encoded thereby the cell membrane fusion-generating activity;
incorporating into the vector a nucleic acid sequence encoding a gene product comprising cell membrane fusion-generating activity;
or both.

44. The vector of claim 39, wherein said incorporating step is further defined as:
providing a first polynucleotide comprising a Herpes Simplex Virus genome, said Herpes Simplex Virus being non-infectious;
providing a second polynucleotide comprising:
the nucleic acid sequence encoding at least one gene product comprising cell membrane fusion-generating activity; and
at least one nucleic acid sequence encoding a gene product comprising a functional packaging signal; and
incorporating the nucleic acid sequence encoding a gene product comprising cell membrane fusion-generating activity and the nucleic acid sequence encoding a gene product comprising a functional packaging signal into the first polynucleotide, wherein said incorporating step generates an infectious Herpes Simplex Virus.

45. The vector of claim 40, wherein the incorporating the nucleic acid sequence encoding a gene product comprising cell membrane fusion-generating activity and the nucleic acid sequence encoding a gene product comprising a functional packaging signal into the first polynucleotide step is further defined as:
mixing the first and second polynucleotides together to form a mixture;
introducing the mixture to a cell; and
assaying for lysis of said cell.

46. The vector of claim 40, wherein the first polynucleotide is provided on a bacterial artificial chromosome.

47. The vector of claim 40, wherein the Herpes Simplex Virus of the first polynucleotide comprises:
a deletion of γ34.5;
a deletion of one or more copies of pac; or
a combination thereof.

48. The vector of claim 40, wherein said infectious Herpes Simplex Virus is replication selective.

49. The vector of claim 40, wherein the second polynucleotide is provided on a plasmid.

50. The vector of claim 40, wherein the expression of the nucleic acid sequence encoding at least one gene product comprising cell membrane fusion-generating activity is regulated by CMV immediate early promoter.

51. A method of generating a vector comprising a first cell membrane fusion-generating activity and a second cell membrane fusion-generating activity, comprising at least one of the following steps:
generating a mutation in a nucleic acid sequence of the vector, wherein the mutation confers to the vector or a gene product encoded thereby the cell membrane fusion-generating activity;
incorporating into the vector a nucleic acid sequence encoding a gene product comprising cell membrane fusion-generating activity;
or both.

52. The method of claim 47, wherein said incorporating step is further defined as:
providing a first polynucleotide comprising a Herpes Simplex Virus genome, said Herpes Simplex Virus being non-infectious;
providing a second polynucleotide comprising:
the nucleic acid sequence encoding at least one gene product comprising cell membrane fusion-generating activity; and
at least one nucleic acid sequence encoding a gene product comprising a functional packaging signal; and
incorporating the nucleic acid sequence encoding a gene product comprising cell membrane fusion-generating activity and the nucleic acid sequence encoding a gene product comprising a functional packaging signal into the first polynucleotide, wherein said incorporating step generates an infectious Herpes Simplex Virus.

53. A vector obtained by the method of claim 47.

54. A method of increasing tumor antigen presentation in an individual, said individual comprising a malignant cell, comprising the step of providing to the individual a vector comprising a first cell membrane fusion-generating activity and a second cell membrane fusion-generating activity.

55. The method of claim 54, wherein said increased tumor antigen presentation provides an improved antitumor immunity in the individual compared to in the absence of said increased tumor antigen presentation.
